(19) United States
(12) Patent Application Publication
(10) Pub. No.: US 2017/0200904 A1
(43) Pub. Date: Jul. 13, 2017

(54) AN ORGANIC ELECTROLUMINESCENT COMPOUND AND AN ORGANIC ELECTROLUMINESCENT DEVICE COMPRISING THE SAME

(71) Applicant: Rohm and Haas Electronic Materials Korea Ltd., Cheonan (KR)

(72) Inventors: Su-Hyun Lee, Suwon (KR); Chi-Sik Kim, Hwaseong (KR); Ji-Song Jun, Hwaseong (KR); Kyoung-Jin Park, Seongnam (KR); Yoo-Jin Doh, Seoul (KR)

(73) Assignee: Rohm and Haas Electronic Materials Korea Ltd., Cheonan (KR)

(21) Appl. No.: 15/314,515

(22) PCT Filed: Jun. 9, 2015
(86) PCT No.: PCT/KR2015/005744

§ 371 Date: Nov. 29, 2016

(30) Foreign Application Priority Data
Jun. 9, 2014 (KR) 10-2014-0069488
Jun. 8, 2015 (KR) 10-2015-0080294

(51) Int. Cl. H01L 51/00; C07D 405/14; C07D 403/10; C07D 409/14; C07D 403/04; C09K 11/02

(52) U.S. Cl. CPC H01L 51/0072; C07D 403/04; H01L 51/0067; C09K 11/025; C07D 403/10; C07D 409/14; H01L 51/0074; C07D 405/14; H01L 51/0073

(57) ABSTRACT

The present invention relates to an organic electroluminescent compound and an organic electroluminescent device comprising the same. By using the organic electroluminescent compound according to the present invention, it is possible to produce an organic electroluminescent device having improved lifespan characteristics.

### TECHNICAL FIELD

The present invention relates to organic electroluminescent compounds and organic electroluminescent device comprising the same.

### BACKGROUND ART

An electroluminescent device (EL device) is a self-light-emitting device which has advantages in that it provides a wider viewing angle, a greater contrast ratio, and a faster response time. The first organic EL device was developed by Eastman Kodak, by using small aromatic diamine molecules, and aluminum complexes as materials for forming a light-emitting layer [Appl. Phys. Lett. 51, 913, 1987].

The most important factor determining luminous efficiency in an organic EL device is light-emitting materials. Until now, fluorescent materials have been widely used as light-emitting material. However, in view of electroluminescent mechanisms, since phosphorescent materials theoretically enhance luminous efficiency by four (4) times compared to fluorescent materials, development of phosphorescent light-emitting materials are widely being researched. Iridium(III) complexes have been widely known as phosphorescent materials, including bis(2-(2′-benzothienyl)-pyridinato-N,C3′)iridium(acetylacetonate) ((acac)Ir(btp)₂), tris(2-phenylpyridine)iridium (Ir(ppy)₃) and bis(4,6-difluorophenylpyridinato-N,C2)picolinate iridium (Firpic) as red, green, and blue materials, respectively.

At present, 4,4′-N,N′-dicarbazol-biphenyl (CBP) is the most widely known phosphorescent host material. Recently, Pioneer (Japan) et al. Developed a high performance organic EL device using bathocuproine (BCP) and aluminum(lll)bis(2-methyl-8-quinolinate)(4-phenylphenolate) (BAlq) etc., as host materials, which were known as hole blocking layer materials.

Although these materials provide good light-emitting characteristics, they have the following disadvantages: (1) Due to their low glass transition temperature and poor thermal stability, their degradation may occur during a high-temperature deposition process in a vacuum, and the lifespan of the device decreases. (2) The power efficiency of an organic EL device is given by [(π/voltage)×current efficiency], and the power efficiency is inversely proportional to the voltage. Although an organic EL device comprising phosphorescent host materials provides higher current efficiency (cd/A) than one comprising fluorescent materials, a significantly high driving voltage is necessary. Thus, there is no merit in terms of power efficiency (Im/W). (3) Further, the operational lifespan of an organic EL device is short and luminous efficiency is still required to be improved. Meanwhile, in order to enhance its efficiency and stability, an organic EL device has a structure of a multilayer comprising a hole injection layer, a hole transport layer, a light-emitting layer, an electron transport layer, and an electron injection layer. The selection of a compound comprised in the hole transport layer is known as a method for improving the characteristics of a device such as hole transport efficiency to the light-emitting layer, luminous efficiency, lifespan, etc.

In this regard, copper phthalocyanine (CuPc), 4,4′-bis[N-(1-naphthyl)-N-phenylamino]biphenyl (NPB), N,N′-diphenyl-N,N′-bis(3-methylphenyl)-(1,1′-biphenyl)-4,4′-diamine (TPD), 4,4′,4″-tris(3-methylphenylphenylamino)triphenylamine (MTDATA), etc., were used as a hole injection and transport material. However, an organic EL device using these materials is problematic in quantum efficiency and operational lifespan. It is because, when an organic EL device is driven under high current, thermal stress occurs between an anode and the hole injection layer. Thermal stress significantly reduces the operational lifespan of the device. Further, since the organic material used in the hole injection layer has very high hole mobility, the hole-electron charge balance may be broken and quantum yield (cd/A) may decrease.

Therefore, a hole transport layer for improving durability of an organic EL device still needs to be developed.

Korean Patent Appln. Laying-Open No. 2010-0130197 discloses a compound wherein a nitrogen-containing heteroaryl group such as triazine is bonded to the nitrogen atom of a carbazole fused with indene as an organic electroluminescent compound. However, the organic electroluminescent device disclosed in the above reference is not satisfactory in terms of lifespan characteristic.

### DISCLOSURE OF THE INVENTION

### Problems to be Solved

The objective of the present invention is to provide i) an organic electroluminescent compound which can produce an organic electroluminescent device having excellent lifespan characteristic, and ii) an organic electroluminescent device comprising the compound.

### Solution to Problems

The present inventors found that the above objective can be achieved by an organic electroluminescent compound represented by the following formula 1:

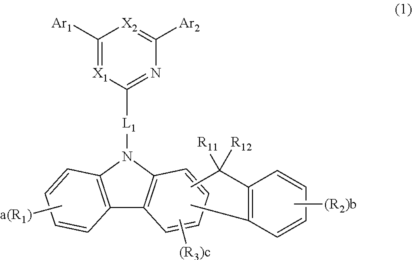

wherein

X₁ and X₂ each independently represent CH or N;

L₁ represents a single bond, a substituted or unsubstituted (C6-C30)arylene, or a substituted or unsubstituted 5- to 30-membered heteroarylene;

Ar₁ represents a substituted or unsubstituted (C6-C18)aryl;

Ar₂ represents a substituted or unsubstituted (C6-C18)aryl, or a substituted or unsubstituted 5- to 15-membered heteroaryl;

Ar₁ and Ar₂ are different from each other;

R₁ and R₂ each independently represent hydrogen, deuterium, a halogen, a cyano, a substituted or unsubstituted (C1-C30)alkyl, a substituted or unsubstituted (C6-C30)aryl, a substituted or unsubstituted 5- to 30-membered heteroaryl, a substituted or unsubstituted (C6-C30)aryl(C1-C30)alkyl, a substituted or unsubstituted (C3-C30)cycloalkyl, a substituted or unsubstituted (C1-C30)alkoxy, a substituted or unsubstituted (C1-C30)alkylsilyl, a substituted or unsubstituted (C6-C30)arylsilyl, a substituted or unsubstituted (C6-C30)aryl(C1-C30)alkylsilyl, a substituted or unsubstituted (C1-C30)alkylamino, a substituted or unsubstituted (C6-C30)arylamino, or a substituted or unsubstituted (C1-C30)alkyl(C6-C30)arylamino; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur;

R₃ represents hydrogen, deuterium, a halogen, a cyano, a substituted or unsubstituted (C1-C30)alkyl, a substituted or unsubstituted (C6-C30)aryl, or a substituted or unsubstituted 5- to 30-membered heteroaryl; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur;

R₁₁ and R₁₂ each independently represent a substituted or unsubstituted (C1-C30)alkyl, a substituted or unsubstituted (C6-C30)aryl, or a substituted or unsubstituted 5- to 30-membered heteroaryl; or are linked to each other to form a mono- or polycyclic (C3-C30) alicyclic or aromatic ring, whose carbon atom(s) may be replaced with at least one hetero atom selected from nitrogen, oxygen, and sulfur;

a and b each independently represent an integer of 1 to 4, where a or b is an integer of 2 or more, each of R₁ and each of R₂ may be the same or different;

c is an integer of 1 to 2, where c is 2, each of R₃ may be the same or different; and

the heteroaryl(ene) contains at least one hetero atom selected from B, N, O, S, Si, and P.

### Effects of the Invention

By using the organic electroluminescent compound according to the present invention, it is possible to manufacture an organic electroluminescent device having improved lifespan characteristics.

### EMBODIMENTS OF THE INVENTION

Hereinafter, the present invention will be described in detail. However, the following description is intended to explain the invention, and is not meant in any way to restrict the scope of the invention.

The present invention relates to an organic electroluminescent compound of formula 1, an organic electroluminescent material comprising the compound, and an organic electroluminescent device comprising the material.

Generally, in order to improve the thermal stability of an organic electroluminescent device, Tg (glass transition temperature) of a host compound used for the light-emitting material can be increased. As a means to increase Tg, various substituents can be introduced to the host compound. However, if substituents are introduced excessively, the deposition temperature of the host compound becomes too high, and materials degrade or become damaged during the deposition process. Thus, substituents should be introduced in an appropriate level to obtain appropriate Tg, and a molecular weight should be controlled to maintain a low deposition temperature. Accordingly, the present invention solves the problem by bonding unsymmetrically two substituents to a nitrogen-containing heterocyclic ring which determines the LUMO (lowest unoccupied molecular orbital) energy level. More specifically, there is a problem that when symmetrically introducing phenyl groups having a low molecular weight as substituents, thermal stability can be obtained, but the device characteristics deteriorate. In addition, when aryl or heteroaryl groups having higher molecular weights than phenyl groups are bonded symmetrically to improve the device characteristics, thermal stability is not satisfactory. Therefore, in order to improve both device characteristics and thermal stability, substituents are bonded unsymmetrically to a nitrogen-containing heterocyclic ring so that crystallinity decreases, the device characteristics are improved, and the thermal stability is improved due to a low molecular weight.

Hereinafter, the organic electroluminescent compound represented by formula 1 will be described in detail.

The compound of formula 1 may be represented by one of the following formulae 2 to 7:

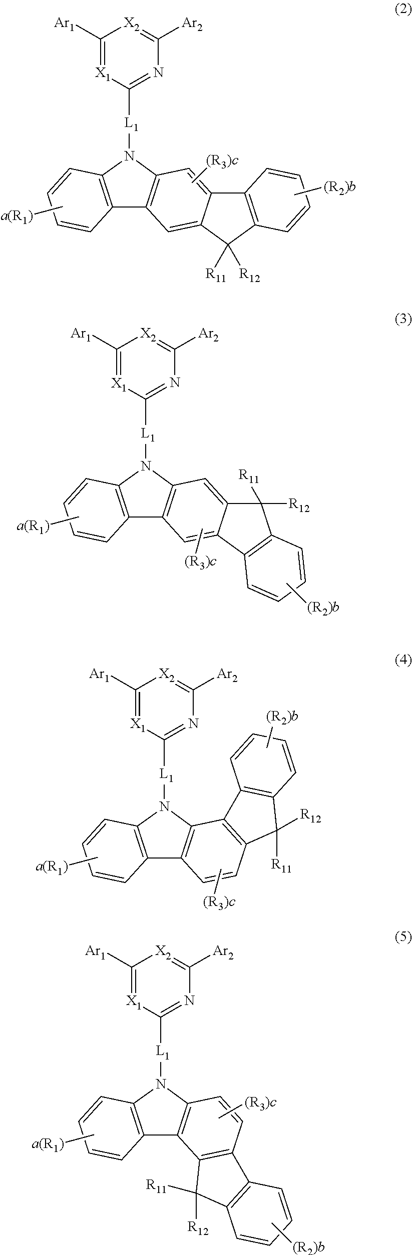

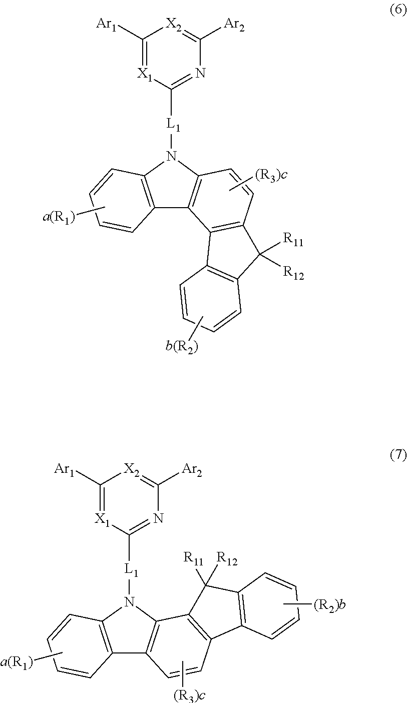

wherein

X₁, X₂, L₁, Ar₁, Arₑ, R₁ to R₃, R₁₁, R₁₂, a, b, and c are as defined in formula 1.

Herein, “(C1-C30)alkyl” is meant to be a linear or branched alkyl having 1 to 30 carbon atoms, in which the number of carbon atoms is preferably 1 to 10, more preferably 1 to 6, and includes methyl, ethyl, n-propyl, isopropyl, n-butyl, isobutyl, tert-butyl, etc.; “(C2-C30)alkenyl” is meant to be a linear or branched alkenyl having 2 to 30 carbon atoms, in which the number of carbon atoms is preferably 2 to 20, more preferably 2 to 10, and includes vinyl, 1-propenyl, 2-propenyl, 1-butenyl, 2-butenyl, 3-butenyl, 2-methylbut-2-enyl, etc.; “(C2-C30)alkynyl” is meant to be a linear or branched alkynyl having 2 to 30 carbon atoms, in which the number of carbon atoms is preferably 2 to 20, more preferably 2 to 10, and includes ethynyl, 1-propynyl, 2-propynyl, 1-butynyl, 2-butynyl, 3-butynyl, 1-methylpent-2-ynyl, etc.; “(C3-C30)cycloalkyl” is a mono- or polycyclic hydrocarbon having 3 to 30 carbon atoms, in which the number of carbon atoms is preferably 3 to 20, more preferably 3 to 7, and includes cyclopropyl, cyclobutyl, cyclopentyl, cyclohexyl, etc.; “3- to 7-membered heterocycloalkyl” is a cycloalkyl having 3 to 7 ring backbone atoms, including at least one heteroatom selected from B, N, O, S, Si, and P, preferably O, S, and N, and includes tetrahydrofuran, pyrrolidine, thiolan, tetrahydropyran, etc.; “(C6-C30)aryl(ene)” is a monocyclic or fused ring derived from an aromatic hydrocarbon having 6 to 30 carbon atoms, in which the number of carbon atoms is preferably 6 to 20, more preferably 6 to 15, and includes phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, etc.; “5- to 30-membered heteroaryl” is an aryl having 5 to 30 ring backbone atoms, including at least one, preferably 1 to 4 heteroatoms selected from the group consisting of B, N, O, S, Si, and P; is a monocyclic ring, or a fused ring condensed with at least one benzene ring; may be partially saturated; may be one formed by linking at least one heteroaryl or aryl group to a heteroaryl group via a single bond(s); and includes a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl, etc. Further, “halogen” includes F, Cl, Br, and l.

Herein, “substituted” in the expression “substituted or unsubstituted” means that a hydrogen atom in a certain functional group is replaced with another atom or group, i.e. a substituent. The substituents of the substituted (C1-C30)alkyl, the substituted (C3-C30)cycloalkyl, the substituted (C6-C30)aryl(ene), the substituted 5- to 30-membered heteroaryl(ene), the substituted (C6-C30)aryl(C1-C30)alkyl, the substituted (C1-C30)alkoxy, the substituted (C1-C30)alkylsilyl, the substituted (C6-C30)arylsilyl, the substituted (C6-C30)aryl(C1-C30)alkylsilyl, the substituted (C1-C30)alkylamino, the substituted (C6-C30)arylamino, and the substituted (C1-C30)alkyl(C6-C30)arylamino in L₁, Ar₁, Arₑ, R₁ to R₃, R₁₁, and R₁₂ in formula 1 each independently are at least one selected from the group consisting of deuterium, a halogen, a cyano, a carboxyl, a nitro, a hydroxyl, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a 3- to 7-membered heterocycloalkyl, a (C6-C30)aryloxy, a (C6-C30)arylthio, a 5- to 30-membered heteroaryl unsubstituted or substituted with a (C6-C30)aryl, a (C6-C30)aryl unsubstituted or substituted with a 5- to 30-membered heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di- (C1-C30)alkylamino, a mono- or di- (C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl, and preferably each independently are at least one selected from the group consisting of a cyano, a (C1-C6)alkyl, a (C6-C12)aryl, and a 5- to 15-membered heteroaryl.

In formula 1 above, X₁ and X₂ each independently represent CH or N.

L₁ represents a single bond, a substituted or unsubstituted (C6-C30)arylene, or a substituted or unsubstituted 5- to 30-membered heteroarylene, preferably represents a single bond, or a substituted or unsubstituted (C6-C12)arylene, and more preferably represents a single bond, or an unsubstituted (C6-C12)arylene.

Ar₁ represents a substituted or unsubstituted (C6-C18)aryl, and preferably represents a (C6-C18)aryl unsubstituted or substituted with a cyano, a (C1-C6)alkyl, a (C6-C12)aryl, or a 5- to 15-membered heteroaryl.

Ar₂ represents a substituted or unsubstituted (C6-C18)aryl, or a substituted or unsubstituted 5- to 15-membered heteroaryl, and preferably represents a (C6-C18)aryl unsubstituted or substituted with a cyano, a (C1-C6)alkyl, a (C6-C12)aryl, or a 5- to 15-membered heteroaryl; or a 5- to 15-membered heteroaryl unsubstituted or substituted with a (C6-C12)aryl.

According to one embodiment of the present invention, Ar₁ and Ar₂ each independently represent a substituted or unsubstituted phenyl, a substituted or unsubstituted biphenyl, a substituted or unsubstituted terphenyl, a substituted or unsubstituted dibenzothiophene, a substituted or unsubstituted dibenzofuran, a substituted or unsubstituted carbazole, or a substituted or unsubstituted fluorene.

Ar₁ and Ar₂ are different from each other.

R₁ and R₂ each independently represent hydrogen, deuterium, a halogen, a cyano, a substituted or unsubstituted (C1-C30)alkyl, a substituted or unsubstituted (C6-C30)aryl, a substituted or unsubstituted 5- to 30-membered heteroaryl, a substituted or unsubstituted (C6-C30)aryl(C1-C30)alkyl, a substituted or unsubstituted (C3-C30)cycloalkyl, a substituted or unsubstituted (C1-C30)alkoxy, a substituted or unsubstituted (C1-C30)alkylsilyl, a substituted or unsubstituted (C6-C30)arylsilyl, a substituted or unsubstituted (C6-C30)aryl(C1-C30)alkylsilyl, a substituted or unsubstituted (C1-C30)alkylamino, a substituted or unsubstituted (C6-C30)arylamino, or a substituted or unsubstituted (C1-C30)alkyl(C6-C30)arylamino; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur, preferably each independently represent hydrogen, a substituted or unsubstituted (C6-C12)aryl, or a substituted or unsubstituted 5- to 15-membered heteroaryl, and more preferably each independently represent hydrogen, an unsubstituted (C6-C12)aryl, or a 5- to 15-membered heteroaryl unsubstituted or substituted with a (C6-C12)aryl.

R₃ represents hydrogen, deuterium, a halogen, a cyano, a substituted or unsubstituted (C1-C30)alkyl, a substituted or unsubstituted (C6-C30)aryl, or a substituted or unsubstituted 5- to 30-membered heteroaryl; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur, and preferably represents hydrogen.

R₁₁ and R₁₂ each independently represent a substituted or unsubstituted (C1-C30)alkyl, a substituted or unsubstituted (C6-C30)aryl, or a substituted or unsubstituted 5- to 30-membered heteroaryl; or are linked to each other to form a mono- or polycyclic (C3-C30) alicyclic or aromatic ring, whose carbon atom(s) may be replaced with at least one hetero atom selected from nitrogen, oxygen, and sulfur, preferably each independently represent a substituted or unsubstituted (C1-C6)alkyl, or a substituted or unsubstituted (C6-C12)aryl; or are linked to each other to form a mono- or polycyclic (C3-C15) alicyclic or aromatic ring, and more preferably each independently represent an unsubstituted (C1-C6)alkyl, or an unsubstituted (C6-C12)aryl; or are linked to each other to form a mono- or polycyclic (C3-C15) aromatic ring.

According to one embodiment of the present invention, in formula 1 above, X₁ and X₂ each independently represent CH or N; L₁ represents a single bond, or a substituted or unsubstituted (C6-C12)arylene; Ar₁ represents a substituted or unsubstituted (C6-C18)aryl; Ar₂ represents a substituted or unsubstituted (C6-C18)aryl, or a substituted or unsubstituted 5- to 15-membered heteroaryl; Ar₁ and Ar₂ are different from each other; R₁ and R₂ each independently represent hydrogen, a substituted or unsubstituted (C6-C12)aryl, or a substituted or unsubstituted 5- to 15-membered heteroaryl; R₃ represents hydrogen; and R₁₁ and R₁₂ each independently represent a substituted or unsubstituted (C1-C6)alkyl, or a substituted or unsubstituted (C6-C12)aryl; or are linked to each other to form a mono- or polycyclic (C3-C15) alicyclic or aromatic ring.

According to another embodiment of the present invention, in formula 1 above, X₁ and X₂ each independently represent CH or N; L₁ represents a single bond, or an unsubstituted (C6-C12)arylene; Ar₁ represents a (C6-C18)aryl unsubstituted or substituted with a cyano, a (C1-C6)alkyl, a (C6-C12)aryl, or a 5- to 15-membered heteroaryl; Ar₂ represents a (C6-C18)aryl unsubstituted or substituted with a cyano, a (C1-C6)alkyl, a (C6-C12)aryl, or a 5- to 15-membered heteroaryl; or a 5- to 15-membered heteroaryl unsubstituted or substituted with a (C6-C12)aryl; Ar₁ and Ar₂ are different from each other; R₁ and R₂ each independently represent hydrogen, an unsubstituted (C6-C12)aryl, or a 5- to 15-membered heteroaryl unsubstituted or substituted with a (C6-C12)aryl; R₃ represents hydrogen; and R₁₁ and R₁₂ each independently represent an unsubstituted (C1-C6)alkyl, or an unsubstituted (C6-C12)aryl; or are linked to each other to form a mono- or polycyclic (C3-C15) aromatic ring.

The specific compounds of the present invention include the following compounds, but are not limited thereto:

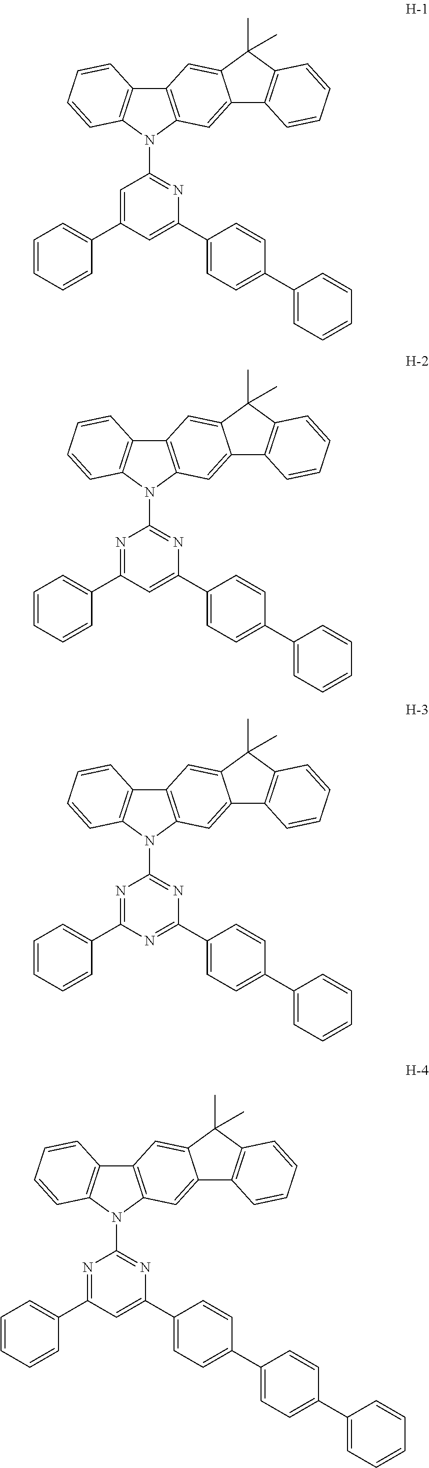 [SMILES: CC1(C)c2ccccc2-c2cc3c(cc21)c1ccccc1n3-c1cc(-c2ccccc2)cc(-c2ccc(-c3ccccc3)cc2)n1 ; CC1(C)c2ccccc2-c2cc3c(cc21)c1ccccc1n3-c1nc(-c2ccccc2)cc(-c2ccc(-c3ccccc3)cc2)n1 ; CC1(C)c2ccccc2-c2cc3c(cc21)c1ccccc1n3-c1nc(-c2ccccc2)nc(-c2ccc(-c3ccccc3)cc2)n1 ; CC1(C)c2ccccc2-c2cc3c(cc21)c1ccccc1n3-c1nc(-c2ccccc2)cc(-c2ccc(-c3ccc(-c4ccccc4)cc3)cc2)n1]

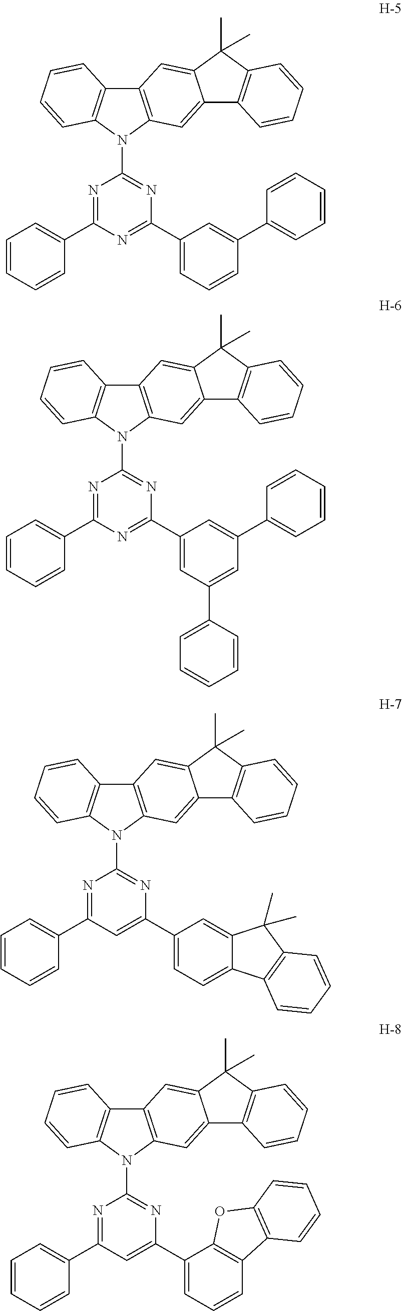

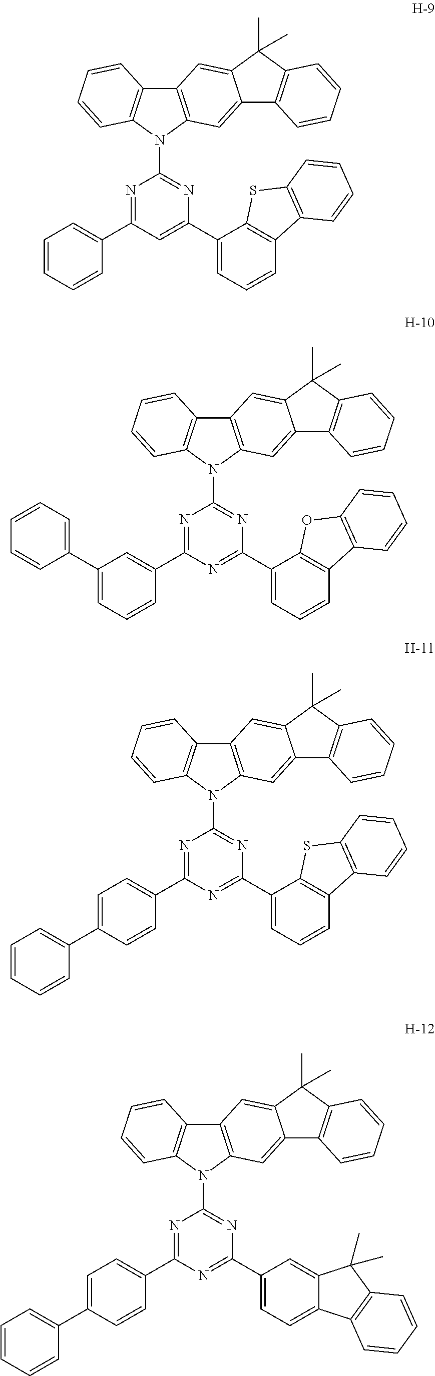 [SMILES: CC1(C)c2ccccc2-c2cc3c(cc21)c1ccccc1n3-c1nc(-c2ccccc2)cc(-c2cccc3c2sc2ccccc23)n1 ; CC1(C)c2ccccc2-c2cc3c(cc21)c1ccccc1n3-c1nc(-c2cccc(-c3ccccc3)c2)nc(-c2cccc3c2oc2ccccc23)n1 ; CC1(C)c2ccccc2-c2cc3c(cc21)c1ccccc1n3-c1nc(-c2ccc(-c3ccccc3)cc2)nc(-c2cccc3c2sc2ccccc23)n1 ; CC1(C)c2ccccc2-c2ccc(-c3nc(-c4ccc(-c5ccccc5)cc4)nc(-n4c5ccccc5c5cc6c(cc54)-c4ccccc4C6(C)C)n3)cc21]

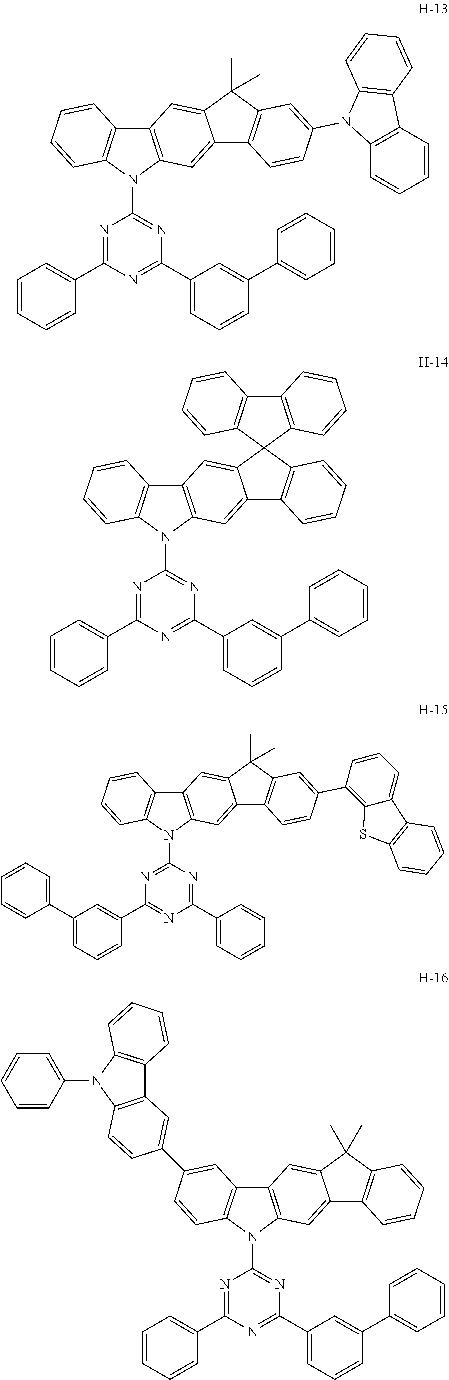

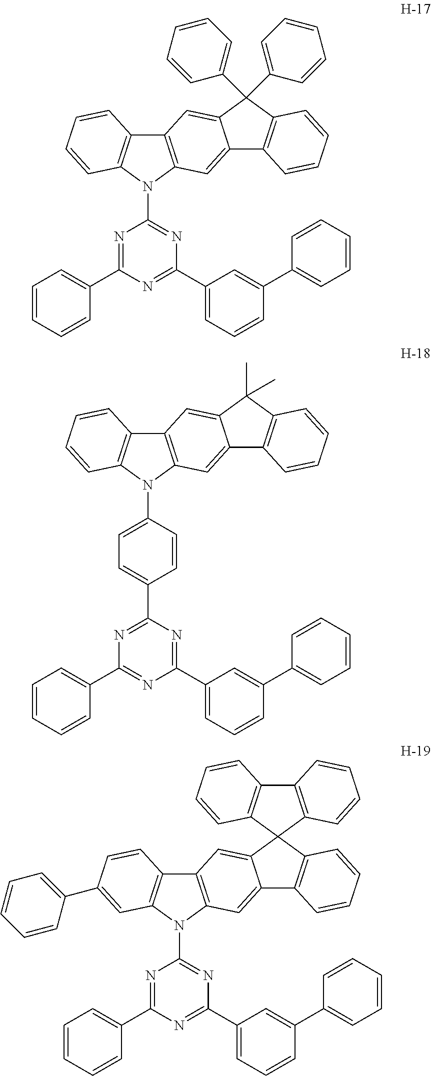

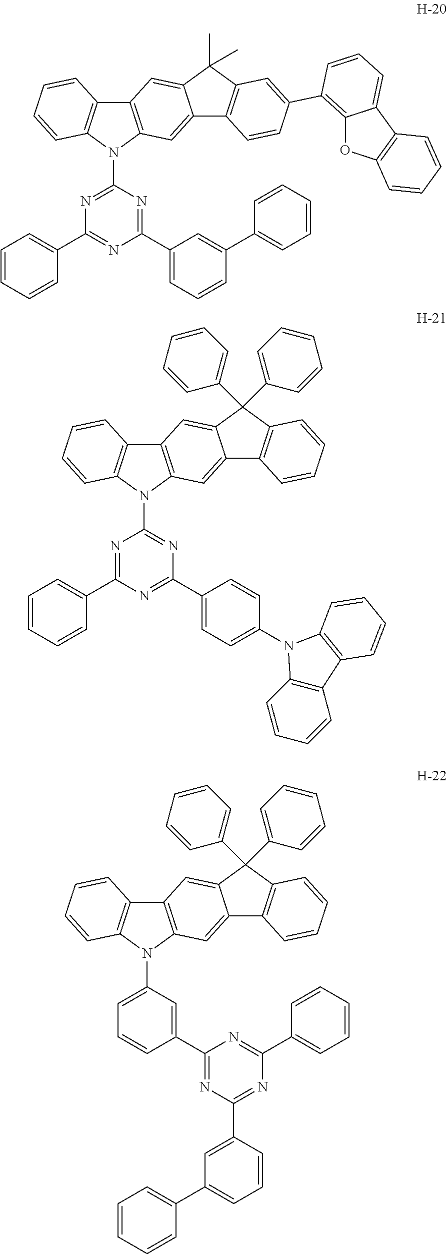

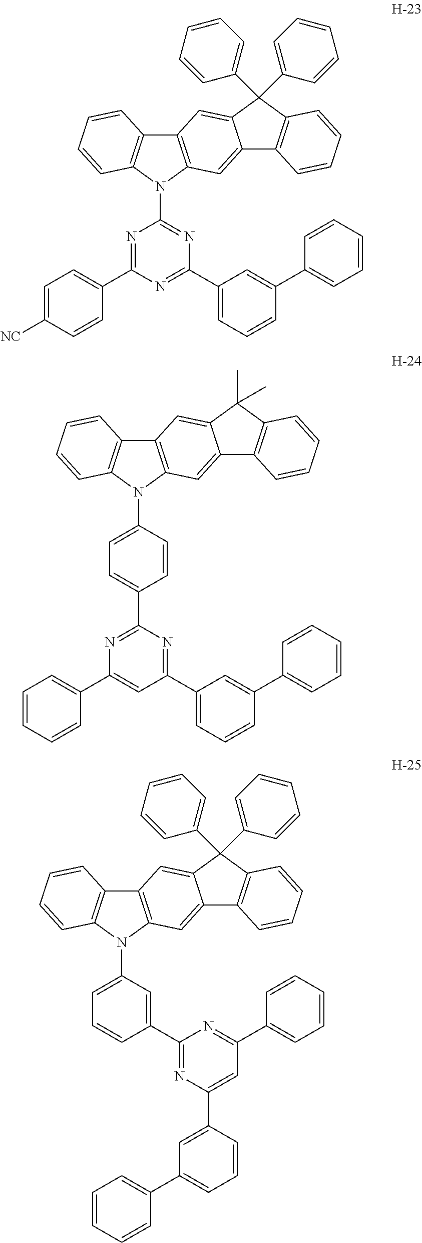

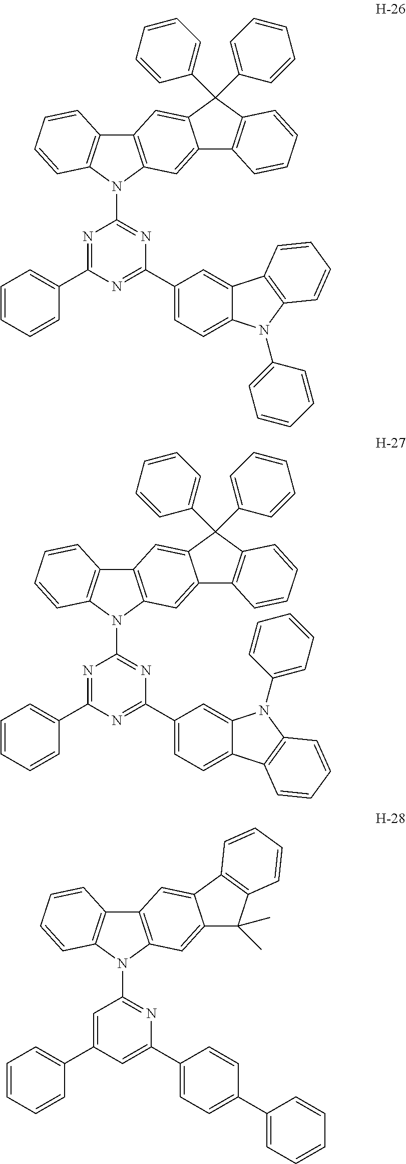

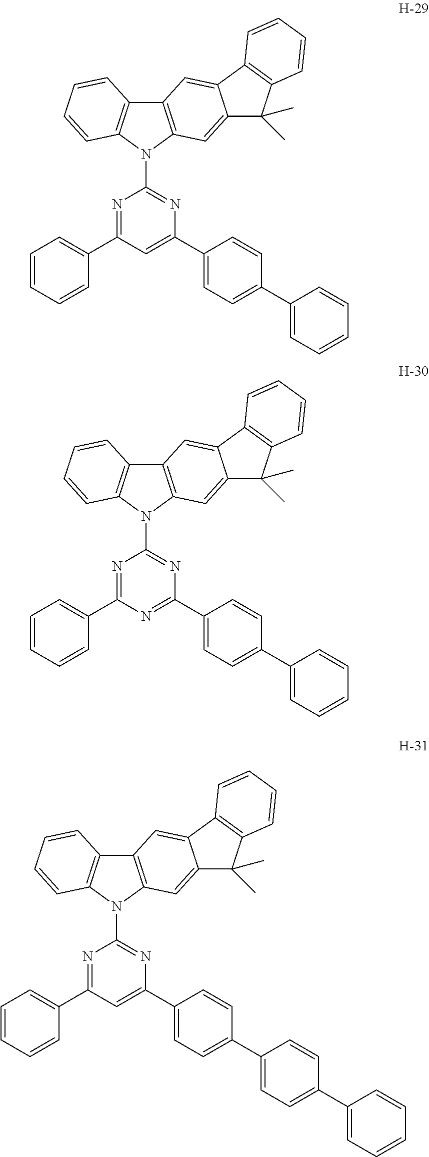

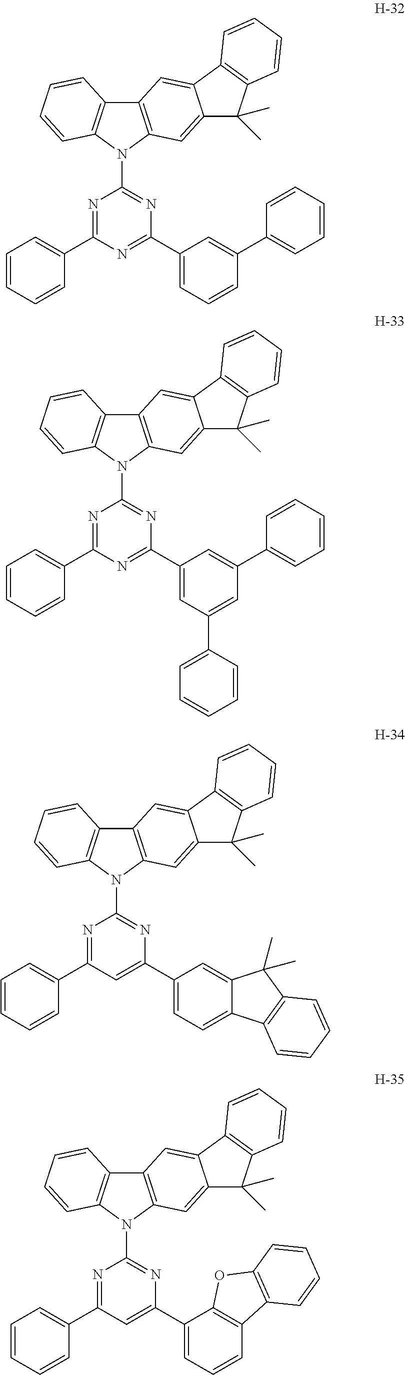

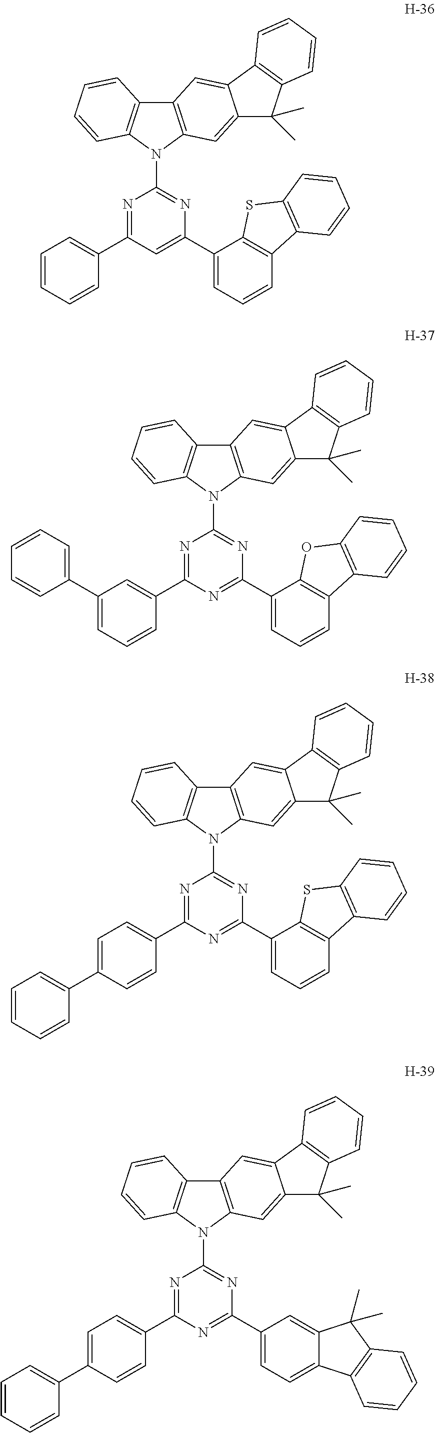 [SMILES: CC1(C)c2ccccc2-c2cc3c4ccccc4n(-c4nc(-c5ccccc5)cc(-c5cccc6c5sc5ccccc56)n4)c3cc21 ; CC1(C)c2ccccc2-c2cc3c4ccccc4n(-c4nc(-c5cccc(-c6ccccc6)c5)nc(-c5cccc6c5oc5ccccc56)n4)c3cc21 ; CC1(C)c2ccccc2-c2cc3c4ccccc4n(-c4nc(-c5ccc(-c6ccccc6)cc5)nc(-c5cccc6c5sc5ccccc56)n4)c3cc21 ; CC1(C)c2ccccc2-c2ccc(-c3nc(-c4ccc(-c5ccccc5)cc4)nc(-n4c5ccccc5c5cc6c(cc54)C(C)(C)c4ccccc4-6)n3)cc21]

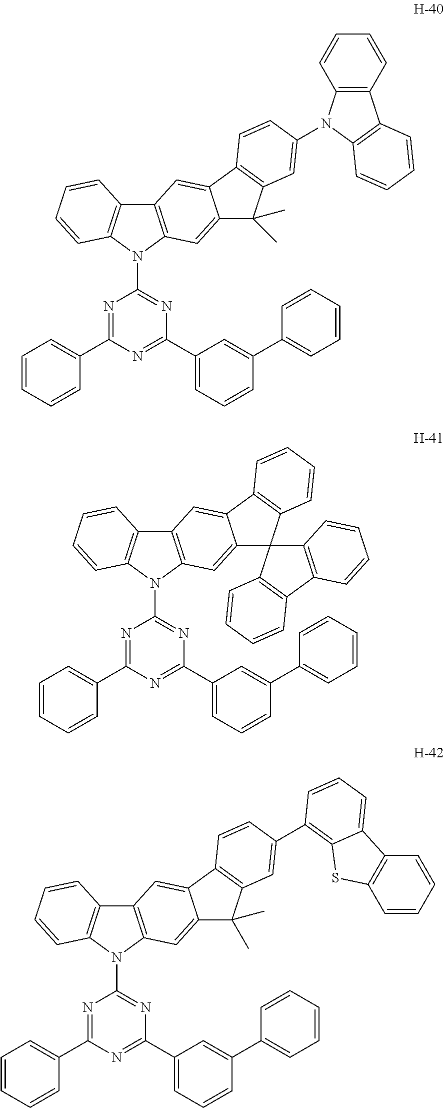 [SMILES: CC1(C)c2cc(-n3c4ccccc4c4ccccc43)ccc2-c2cc3c4ccccc4n(-c4nc(-c5ccccc5)nc(-c5cccc(-c6ccccc6)c5)n4)c3cc21 ; c1ccc(-c2cccc(-c3nc(-c4ccccc4)nc(-n4c5ccccc5c5cc6c(cc54)C4(c5ccccc5-c5ccccc54)c4ccccc4-6)n3)c2)cc1 ; CC1(C)c2cc(-c3cccc4c3sc3ccccc34)ccc2-c2cc3c4ccccc4n(-c4nc(-c5ccccc5)nc(-c5cccc(-c6ccccc6)c5)n4)c3cc21]

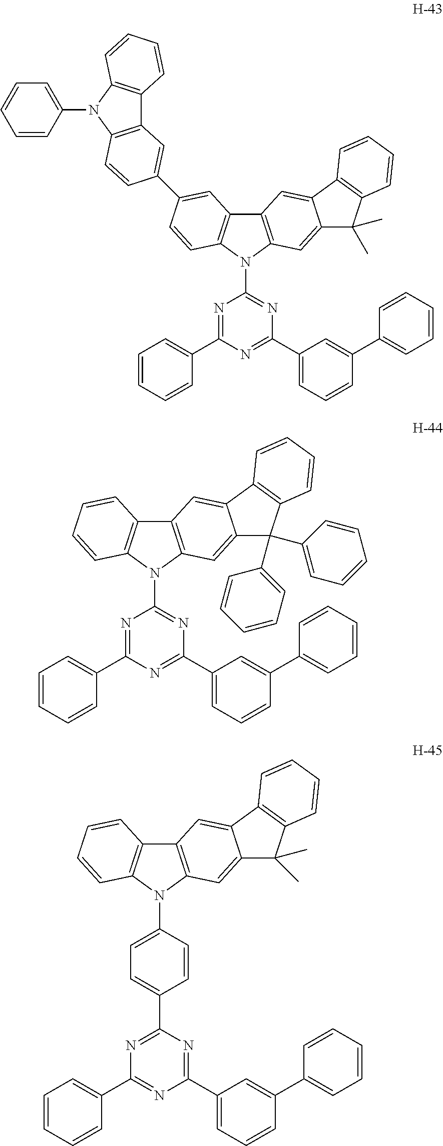 [SMILES: CC1(C)c2ccccc2-c2cc3c4cc(-c5ccc6c(c5)c5ccccc5n6-c5ccccc5)ccc4n(-c4nc(-c5ccccc5)nc(-c5cccc(-c6ccccc6)c5)n4)c3cc21 ; c1ccc(-c2cccc(-c3nc(-c4ccccc4)nc(-n4c5ccccc5c5cc6c(cc54)C(c4ccccc4)(c4ccccc4)c4ccccc4-6)n3)c2)cc1 ; CC1(C)c2ccccc2-c2cc3c4ccccc4n(-c4ccc(-c5nc(-c6ccccc6)nc(-c6cccc(-c7ccccc7)c6)n5)cc4)c3cc21]

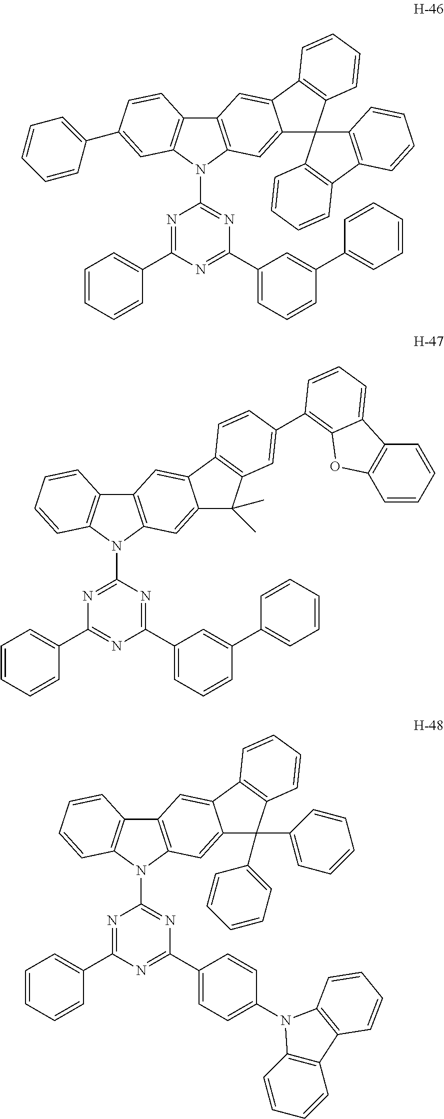 [SMILES: c1ccc(-c2cccc(-c3nc(-c4ccccc4)nc(-n4c5cc(-c6ccccc6)ccc5c5cc6c(cc54)C4(c5ccccc5-c5ccccc54)c4ccccc4-6)n3)c2)cc1 ; CC1(C)c2cc(-c3cccc4c3oc3ccccc34)ccc2-c2cc3c4ccccc4n(-c4nc(-c5ccccc5)nc(-c5cccc(-c6ccccc6)c5)n4)c3cc21 ; c1ccc(-c2nc(-c3ccc(-n4c5ccccc5c5ccccc54)cc3)nc(-n3c4ccccc4c4cc5c(cc43)C(c3ccccc3)(c3ccccc3)c3ccccc3-5)n2)cc1]

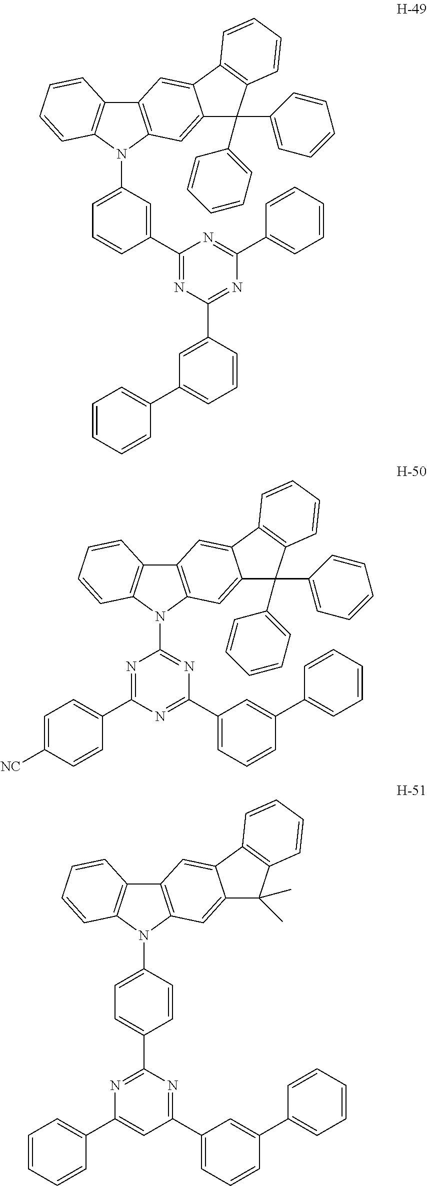

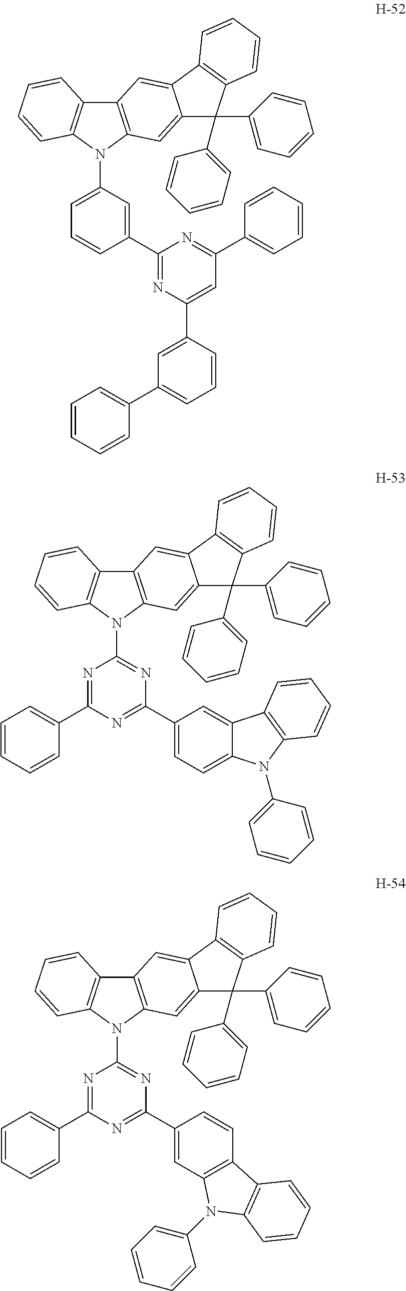

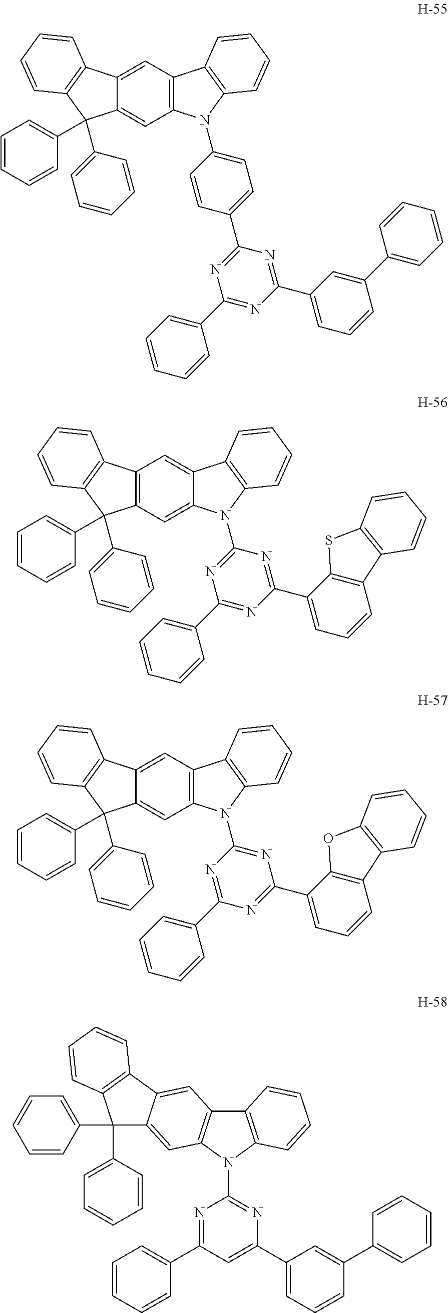 [SMILES: c1ccc(-c2cccc(-c3nc(-c4ccccc4)nc(-c4ccc(-n5c6ccccc6c6cc7c(cc65)C(c5ccccc5)(c5ccccc5)c5ccccc5-7)cc4)n3)c2)cc1 ; c1ccc(-c2nc(-c3cccc4c3sc3ccccc34)nc(-n3c4ccccc4c4cc5c(cc43)C(c3ccccc3)(c3ccccc3)c3ccccc3-5)n2)cc1 ; c1ccc(-c2nc(-c3cccc4c3oc3ccccc34)nc(-n3c4ccccc4c4cc5c(cc43)C(c3ccccc3)(c3ccccc3)c3ccccc3-5)n2)cc1 ; c1ccc(-c2cccc(-c3cc(-c4ccccc4)nc(-n4c5ccccc5c5cc6c(cc54)C(c4ccccc4)(c4ccccc4)c4ccccc4-6)n3)c2)cc1]

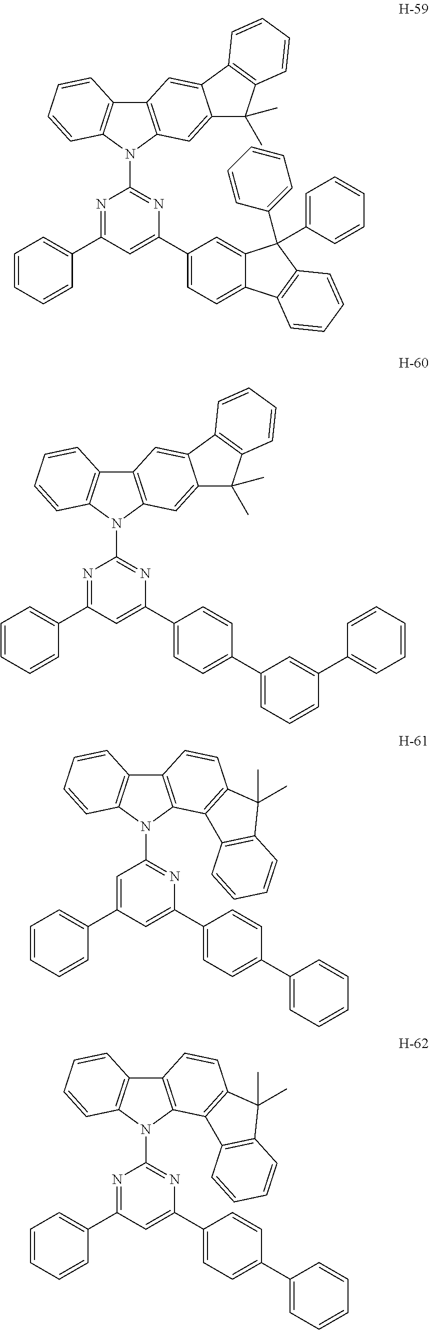 [SMILES: CC1(C)c2ccccc2-c2cc3c4ccccc4n(-c4nc(-c5ccccc5)cc(-c5ccc6c(c5)C(c5ccccc5)(c5ccccc5)c5ccccc5-6)n4)c3cc21 ; CC1(C)c2ccccc2-c2cc3c4ccccc4n(-c4nc(-c5ccccc5)cc(-c5ccc(-c6cccc(-c7ccccc7)c6)cc5)n4)c3cc21 ; CC1(C)c2ccccc2-c2c1ccc1c3ccccc3n(-c3cc(-c4ccccc4)cc(-c4ccc(-c5ccccc5)cc4)n3)c21 ; CC1(C)c2ccccc2-c2c1ccc1c3ccccc3n(-c3nc(-c4ccccc4)cc(-c4ccc(-c5ccccc5)cc4)n3)c21]

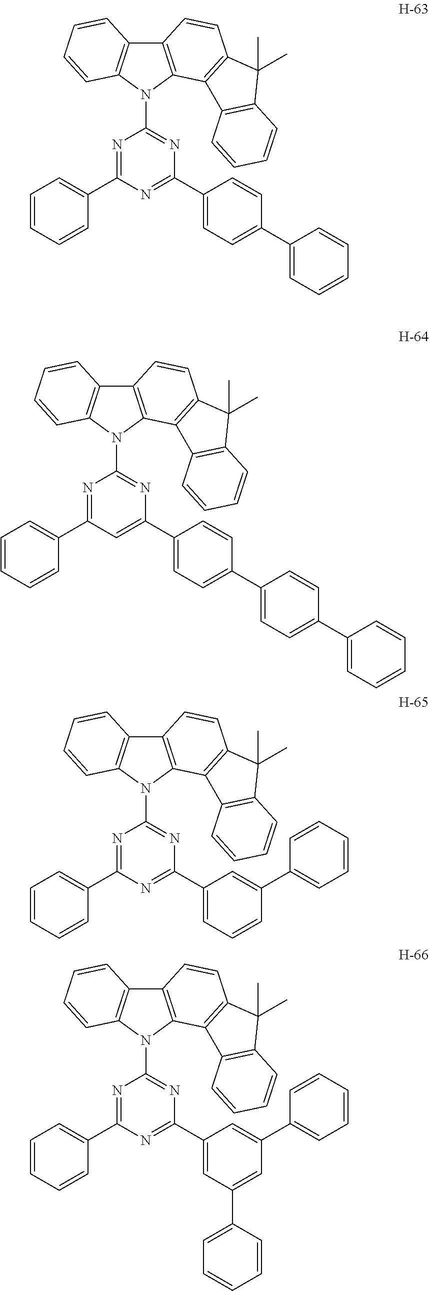

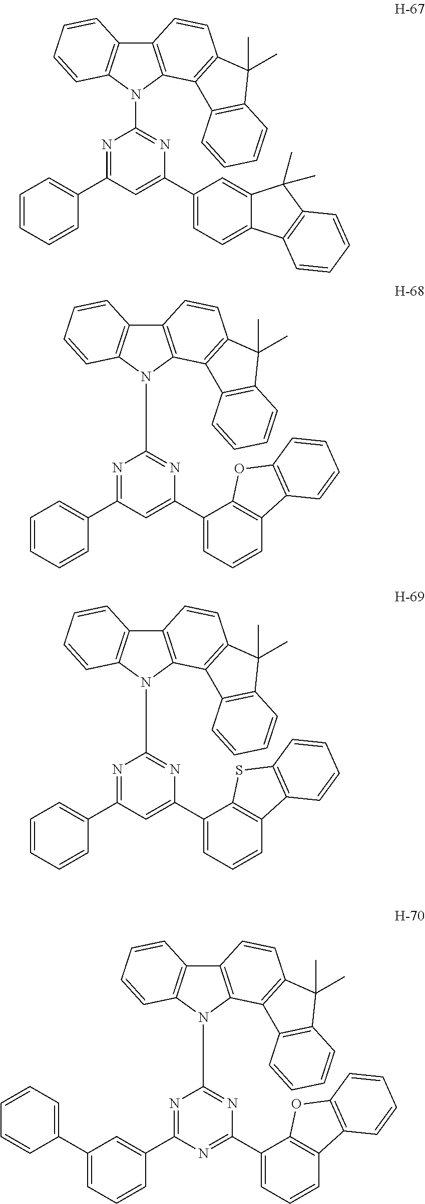 [SMILES: CC1(C)c2ccccc2-c2ccc(-c3cc(-c4ccccc4)nc(-n4c5ccccc5c5ccc6c(c54)-c4ccccc4C6(C)C)n3)cc21 ; CC1(C)c2ccccc2-c2c1ccc1c3ccccc3n(-c3nc(-c4ccccc4)cc(-c4cccc5c4oc4ccccc45)n3)c21 ; CC1(C)c2ccccc2-c2c1ccc1c3ccccc3n(-c3nc(-c4ccccc4)cc(-c4cccc5c4sc4ccccc45)n3)c21 ; CC1(C)c2ccccc2-c2c1ccc1c3ccccc3n(-c3nc(-c4cccc(-c5ccccc5)c4)nc(-c4cccc5c4oc4ccccc45)n3)c21]

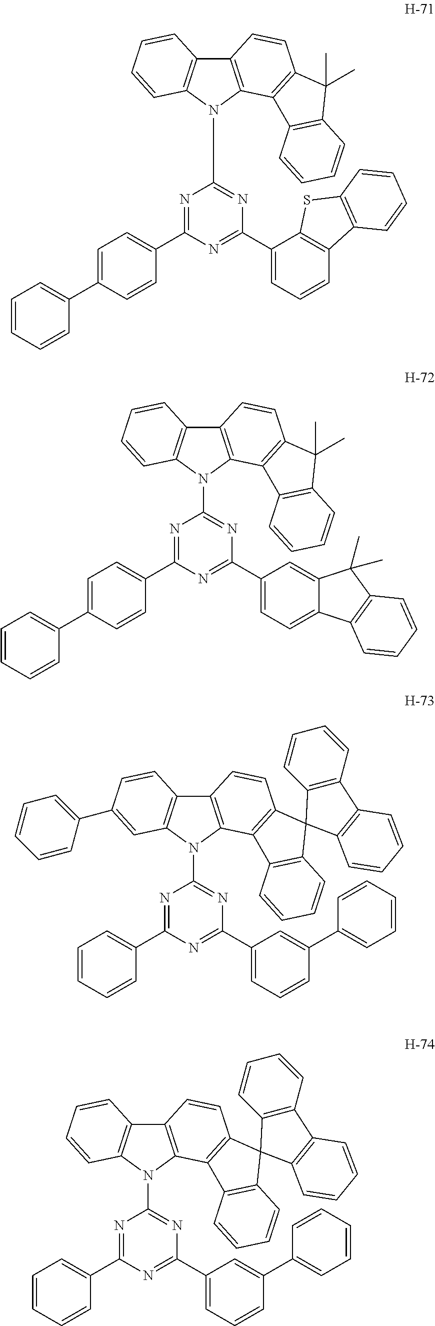 [SMILES: CC1(C)c2ccccc2-c2c1ccc1c3ccccc3n(-c3nc(-c4ccc(-c5ccccc5)cc4)nc(-c4cccc5c4sc4ccccc45)n3)c21 ; CC1(C)c2ccccc2-c2ccc(-c3nc(-c4ccc(-c5ccccc5)cc4)nc(-n4c5ccccc5c5ccc6c(c54)-c4ccccc4C6(C)C)n3)cc21 ; c1ccc(-c2cccc(-c3nc(-c4ccccc4)nc(-n4c5cc(-c6ccccc6)ccc5c5ccc6c(c54)-c4ccccc4C64c5ccccc5-c5ccccc54)n3)c2)cc1 ; c1ccc(-c2cccc(-c3nc(-c4ccccc4)nc(-n4c5ccccc5c5ccc6c(c54)-c4ccccc4C64c5ccccc5-c5ccccc54)n3)c2)cc1]

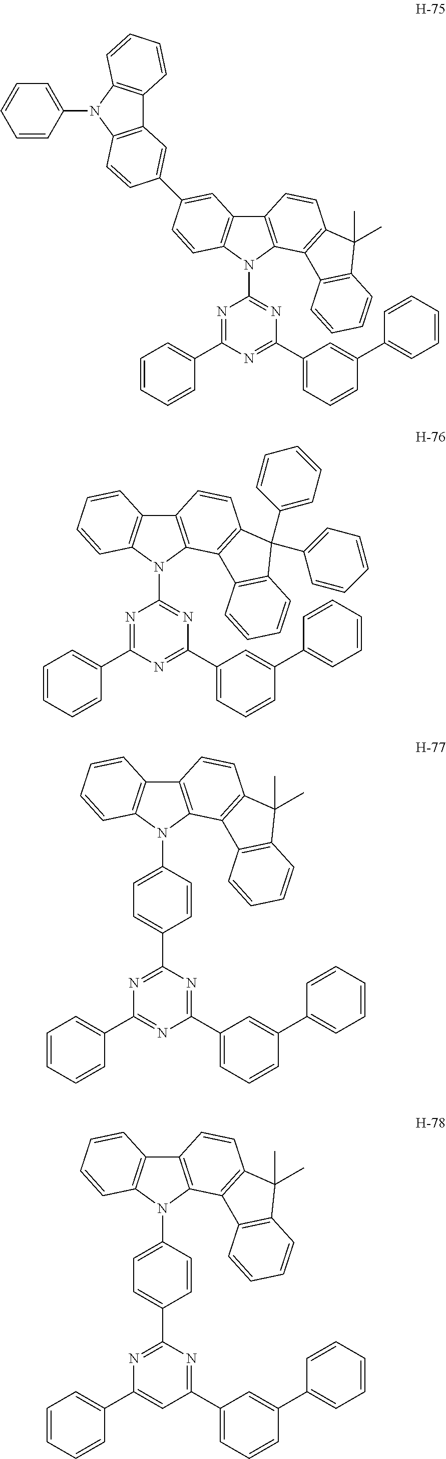 [SMILES: CC1(C)c2ccccc2-c2c1ccc1c3cc(-c4ccc5c(c4)c4ccccc4n5-c4ccccc4)ccc3n(-c3nc(-c4ccccc4)nc(-c4cccc(-c5ccccc5)c4)n3)c21 ; c1ccc(-c2cccc(-c3nc(-c4ccccc4)nc(-n4c5ccccc5c5ccc6c(c54)-c4ccccc4C6(c4ccccc4)c4ccccc4)n3)c2)cc1 ; CC1(C)c2ccccc2-c2c1ccc1c3ccccc3n(-c3ccc(-c4nc(-c5ccccc5)nc(-c5cccc(-c6ccccc6)c5)n4)cc3)c21 ; CC1(C)c2ccccc2-c2c1ccc1c3ccccc3n(-c3ccc(-c4nc(-c5ccccc5)cc(-c5cccc(-c6ccccc6)c5)n4)cc3)c21]

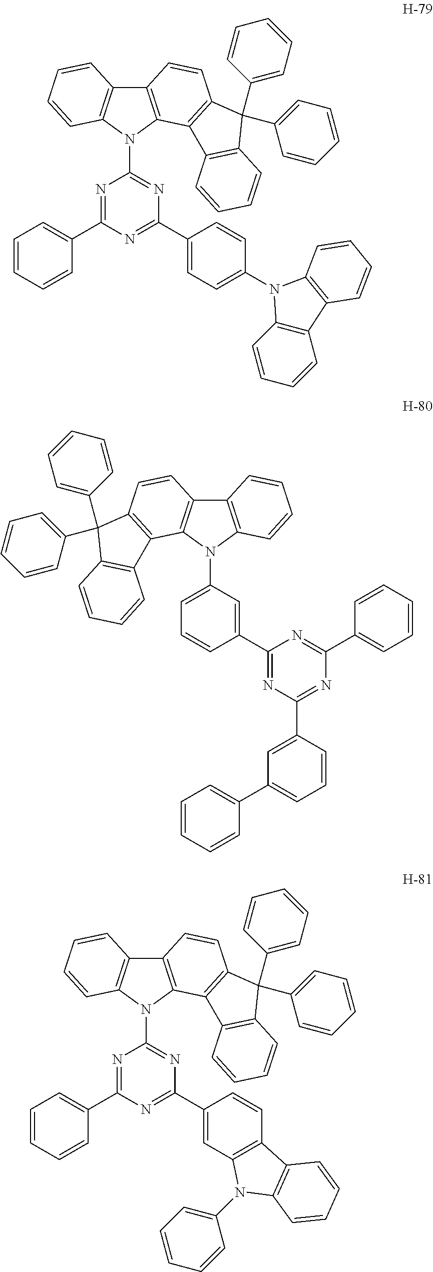 [SMILES: c1ccc(-c2nc(-c3ccc(-n4c5ccccc5c5ccccc54)cc3)nc(-n3c4ccccc4c4ccc5c(c43)-c3ccccc3C5(c3ccccc3)c3ccccc3)n2)cc1 ; c1ccc(-c2cccc(-c3nc(-c4ccccc4)nc(-c4cccc(-n5c6ccccc6c6ccc7c(c65)-c5ccccc5C7(c5ccccc5)c5ccccc5)c4)n3)c2)cc1 ; c1ccc(-c2nc(-c3ccc4c5ccccc5n(-c5ccccc5)c4c3)nc(-n3c4ccccc4c4ccc5c(c43)-c3ccccc3C5(c3ccccc3)c3ccccc3)n2)cc1]

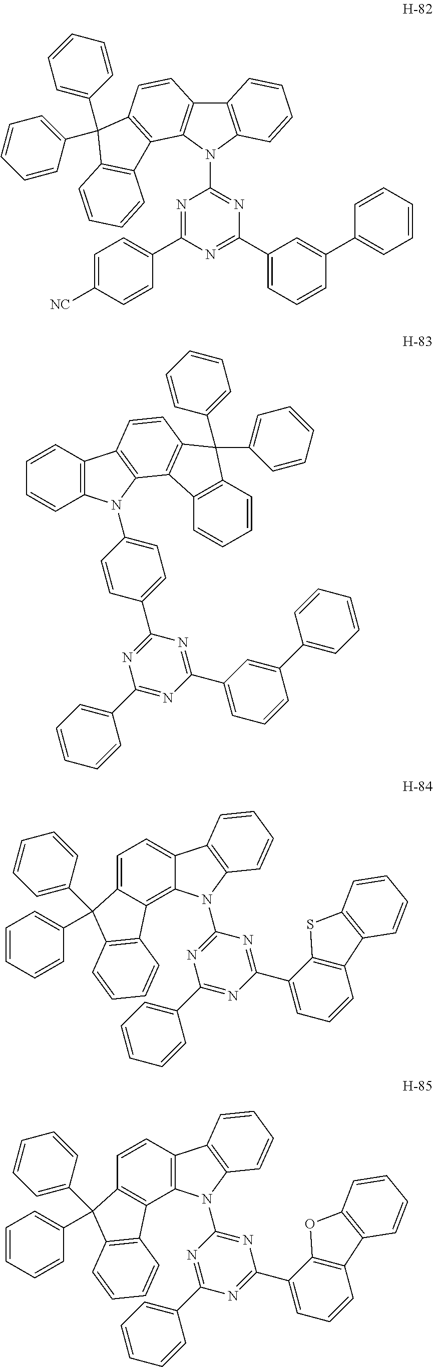 [SMILES: N#Cc1ccc(-c2nc(-c3cccc(-c4ccccc4)c3)nc(-n3c4ccccc4c4ccc5c(c43)-c3ccccc3C5(c3ccccc3)c3ccccc3)n2)cc1 ; c1ccc(-c2cccc(-c3nc(-c4ccccc4)nc(-c4ccc(-n5c6ccccc6c6ccc7c(c65)-c5ccccc5C7(c5ccccc5)c5ccccc5)cc4)n3)c2)cc1 ; c1ccc(-c2nc(-c3cccc4c3sc3ccccc34)nc(-n3c4ccccc4c4ccc5c(c43)-c3ccccc3C5(c3ccccc3)c3ccccc3)n2)cc1 ; c1ccc(-c2nc(-c3cccc4c3oc3ccccc34)nc(-n3c4ccccc4c4ccc5c(c43)-c3ccccc3C5(c3ccccc3)c3ccccc3)n2)cc1]

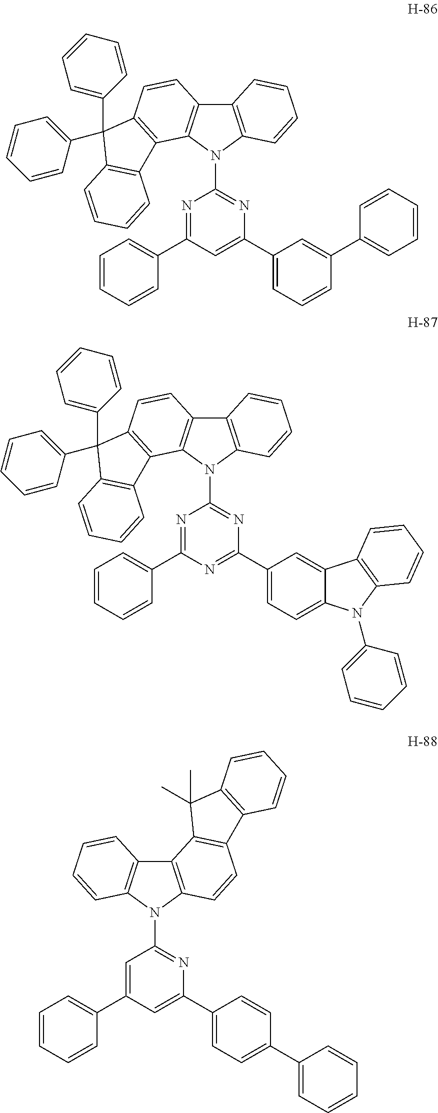 [SMILES: c1ccc(-c2cccc(-c3cc(-c4ccccc4)nc(-n4c5ccccc5c5ccc6c(c54)-c4ccccc4C6(c4ccccc4)c4ccccc4)n3)c2)cc1 ; c1ccc(-c2nc(-c3ccc4c(c3)c3ccccc3n4-c3ccccc3)nc(-n3c4ccccc4c4ccc5c(c43)-c3ccccc3C5(c3ccccc3)c3ccccc3)n2)cc1 ; CC1(C)c2ccccc2-c2ccc3c(c21)c1ccccc1n3-c1cc(-c2ccccc2)cc(-c2ccc(-c3ccccc3)cc2)n1]

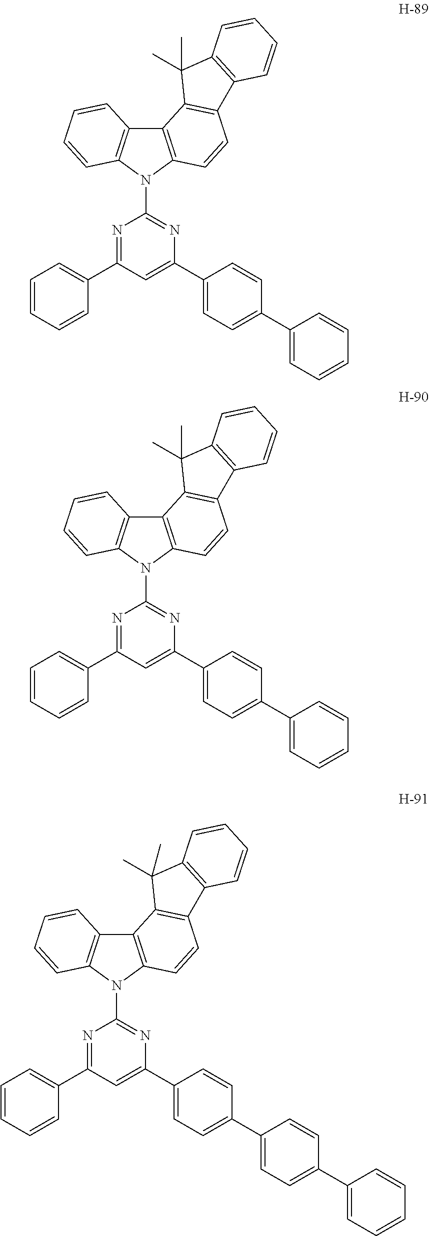 [SMILES: CC1(C)c2ccccc2-c2ccc3c(c21)c1ccccc1n3-c1nc(-c2ccccc2)cc(-c2ccc(-c3ccccc3)cc2)n1 ; CC1(C)c2ccccc2-c2ccc3c(c21)c1ccccc1n3-c1nc(-c2ccccc2)cc(-c2ccc(-c3ccccc3)cc2)n1 ; CC1(C)c2ccccc2-c2ccc3c(c21)c1ccccc1n3-c1nc(-c2ccccc2)cc(-c2ccc(-c3ccc(-c4ccccc4)cc3)cc2)n1]

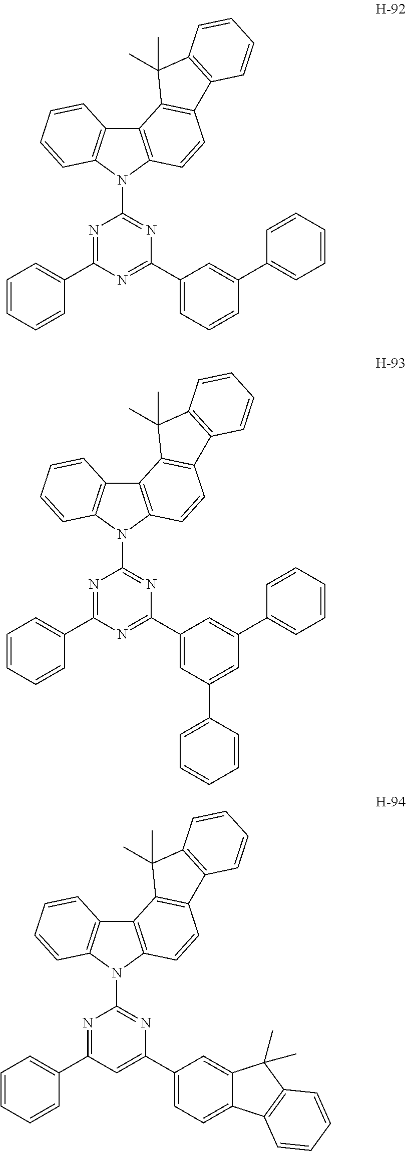

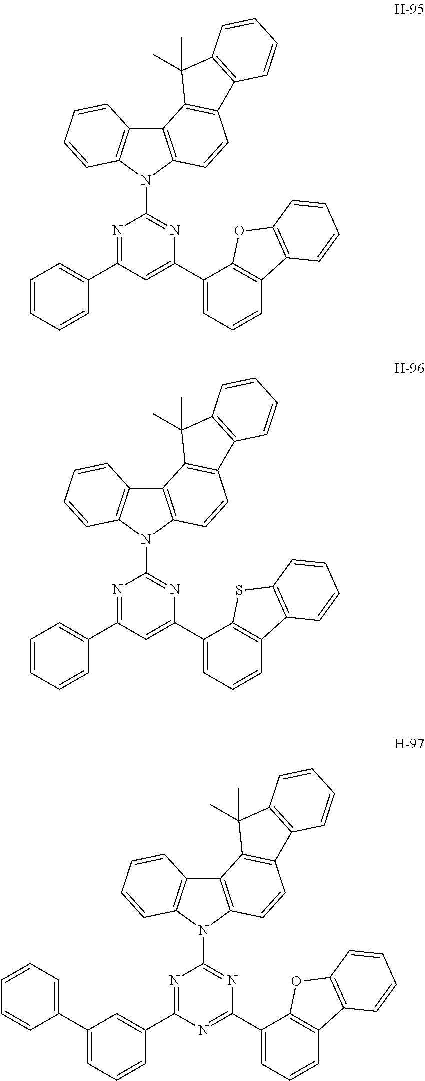 [SMILES: CC1(C)c2ccccc2-c2ccc3c(c21)c1ccccc1n3-c1nc(-c2ccccc2)cc(-c2cccc3c2oc2ccccc23)n1 ; CC1(C)c2ccccc2-c2ccc3c(c21)c1ccccc1n3-c1nc(-c2ccccc2)cc(-c2cccc3c2sc2ccccc23)n1 ; CC1(C)c2ccccc2-c2ccc3c(c21)c1ccccc1n3-c1nc(-c2cccc(-c3ccccc3)c2)nc(-c2cccc3c2oc2ccccc23)n1]

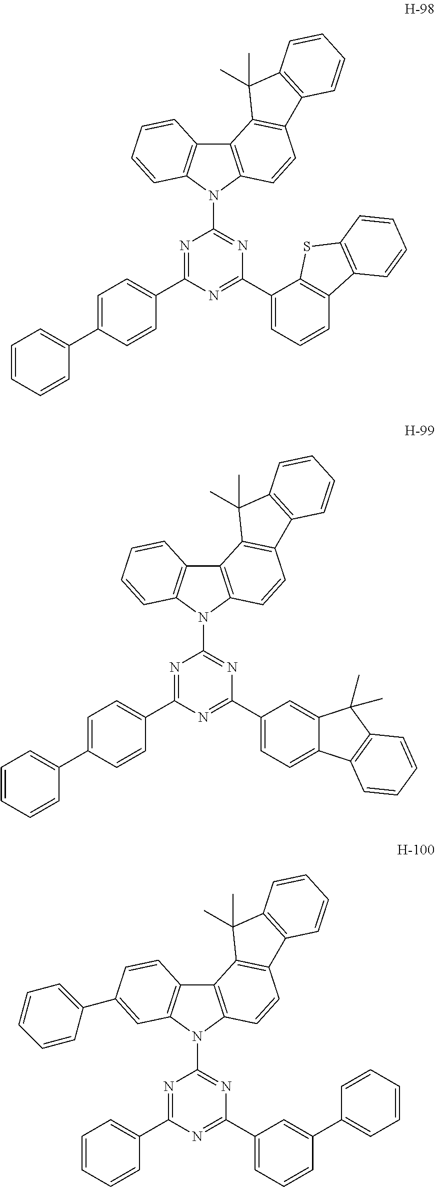 [SMILES: CC1(C)c2ccccc2-c2ccc3c(c21)c1ccccc1n3-c1nc(-c2ccc(-c3ccccc3)cc2)nc(-c2cccc3c2sc2ccccc23)n1 ; CC1(C)c2ccccc2-c2ccc(-c3nc(-c4ccc(-c5ccccc5)cc4)nc(-n4c5ccccc5c5c6c(ccc54)-c4ccccc4C6(C)C)n3)cc21 ; CC1(C)c2ccccc2-c2ccc3c(c21)c1ccc(-c2ccccc2)cc1n3-c1nc(-c2ccccc2)nc(-c2cccc(-c3ccccc3)c2)n1]

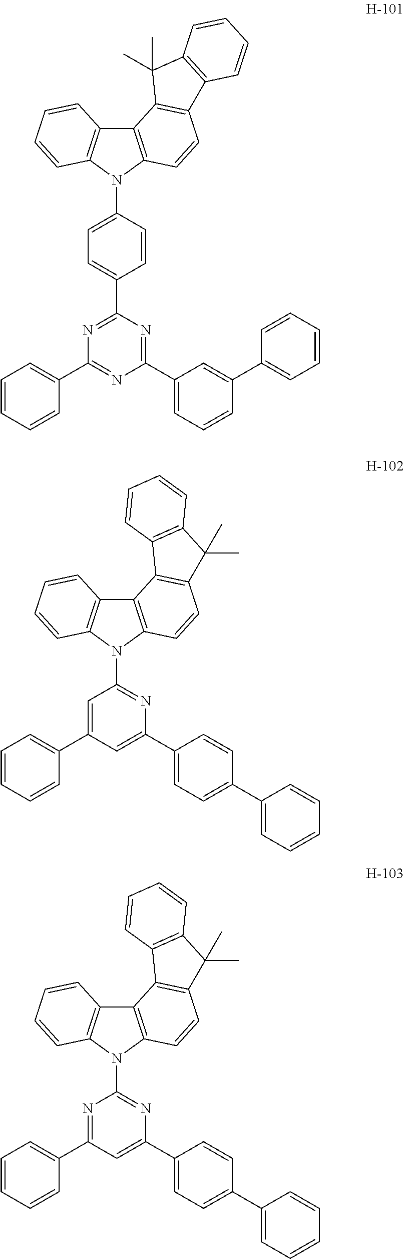 [SMILES: CC1(C)c2ccccc2-c2ccc3c(c21)c1ccccc1n3-c1ccc(-c2nc(-c3ccccc3)nc(-c3cccc(-c4ccccc4)c3)n2)cc1 ; CC1(C)c2ccccc2-c2c1ccc1c2c2ccccc2n1-c1cc(-c2ccccc2)cc(-c2ccc(-c3ccccc3)cc2)n1 ; CC1(C)c2ccccc2-c2c1ccc1c2c2ccccc2n1-c1nc(-c2ccccc2)cc(-c2ccc(-c3ccccc3)cc2)n1]

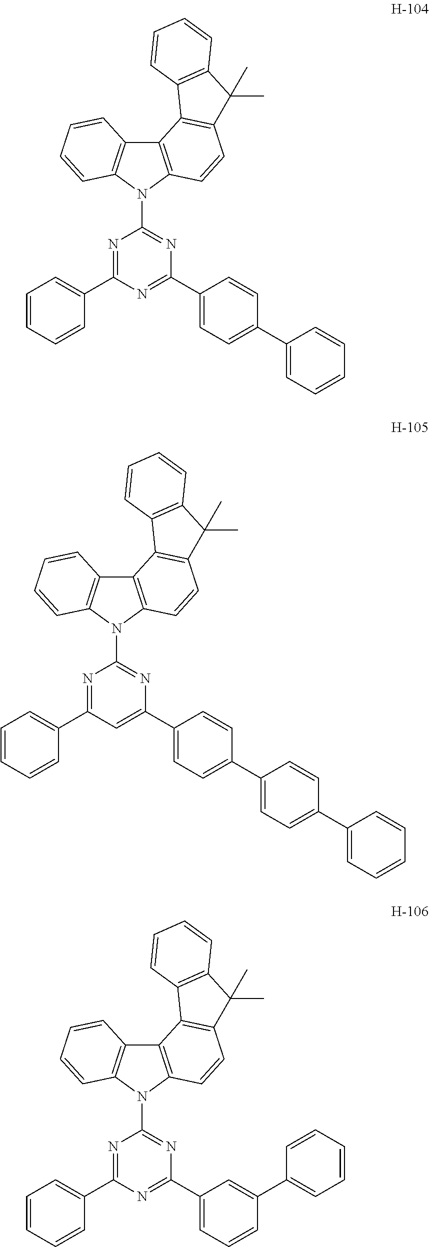 [SMILES: CC1(C)c2ccccc2-c2c1ccc1c2c2ccccc2n1-c1nc(-c2ccccc2)nc(-c2ccc(-c3ccccc3)cc2)n1 ; CC1(C)c2ccccc2-c2c1ccc1c2c2ccccc2n1-c1nc(-c2ccccc2)cc(-c2ccc(-c3ccc(-c4ccccc4)cc3)cc2)n1 ; CC1(C)c2ccccc2-c2c1ccc1c2c2ccccc2n1-c1nc(-c2ccccc2)nc(-c2cccc(-c3ccccc3)c2)n1]

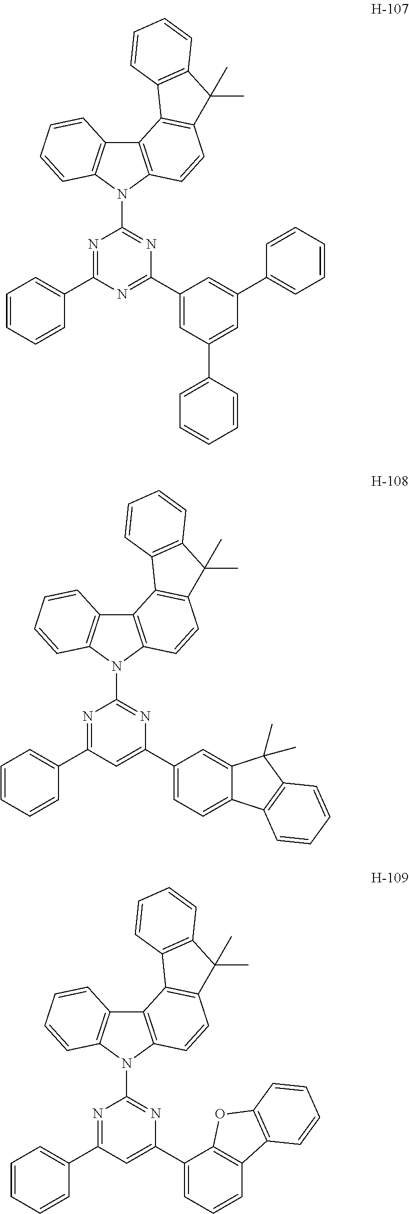 [SMILES: CC1(C)c2ccccc2-c2c1ccc1c2c2ccccc2n1-c1nc(-c2ccccc2)nc(-c2cc(-c3ccccc3)cc(-c3ccccc3)c2)n1 ; CC1(C)c2ccccc2-c2ccc(-c3cc(-c4ccccc4)nc(-n4c5ccccc5c5c6c(ccc54)C(C)(C)c4ccccc4-6)n3)cc21 ; CC1(C)c2ccccc2-c2c1ccc1c2c2ccccc2n1-c1nc(-c2ccccc2)cc(-c2cccc3c2oc2ccccc23)n1]

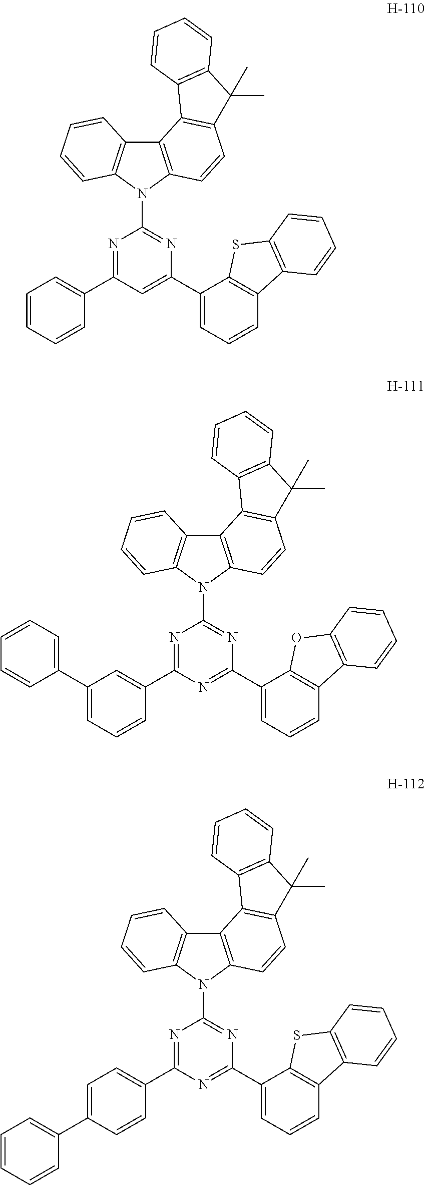 [SMILES: CC1(C)c2ccccc2-c2c1ccc1c2c2ccccc2n1-c1nc(-c2ccccc2)cc(-c2cccc3c2sc2ccccc23)n1 ; CC1(C)c2ccccc2-c2c1ccc1c2c2ccccc2n1-c1nc(-c2cccc(-c3ccccc3)c2)nc(-c2cccc3c2oc2ccccc23)n1 ; CC1(C)c2ccccc2-c2c1ccc1c2c2ccccc2n1-c1nc(-c2ccc(-c3ccccc3)cc2)nc(-c2cccc3c2sc2ccccc23)n1]

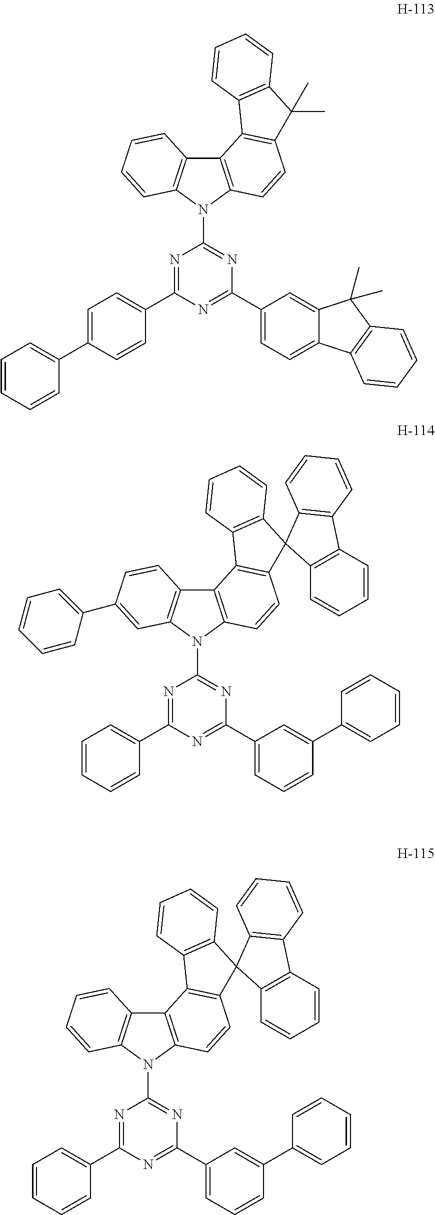 [SMILES: CC1(C)c2ccccc2-c2ccc(-c3nc(-c4ccc(-c5ccccc5)cc4)nc(-n4c5ccccc5c5c6c(ccc54)C(C)(C)c4ccccc4-6)n3)cc21 ; c1ccc(-c2cccc(-c3nc(-c4ccccc4)nc(-n4c5cc(-c6ccccc6)ccc5c5c6c(ccc54)C4(c5ccccc5-c5ccccc54)c4ccccc4-6)n3)c2)cc1 ; c1ccc(-c2cccc(-c3nc(-c4ccccc4)nc(-n4c5ccccc5c5c6c(ccc54)C4(c5ccccc5-c5ccccc54)c4ccccc4-6)n3)c2)cc1]

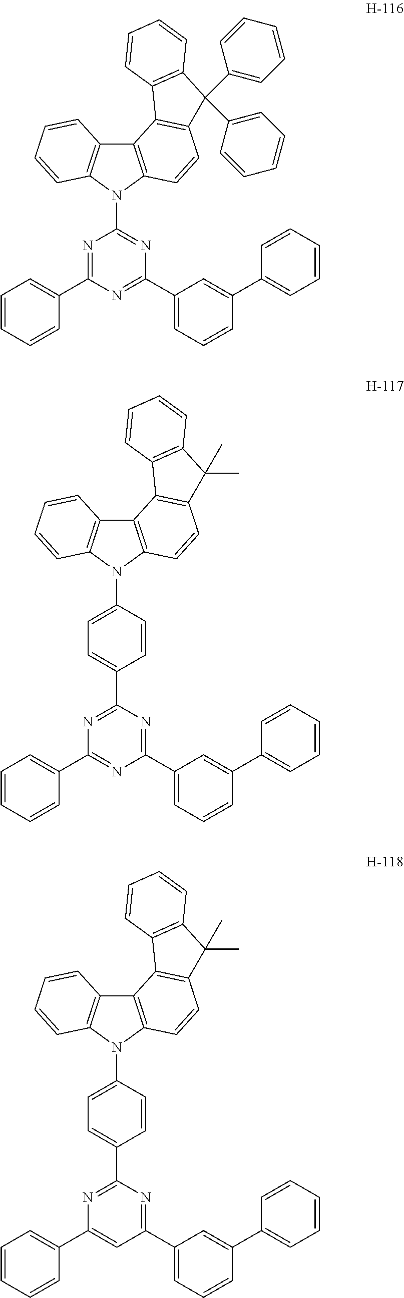 [SMILES: c1ccc(-c2cccc(-c3nc(-c4ccccc4)nc(-n4c5ccccc5c5c6c(ccc54)C(c4ccccc4)(c4ccccc4)c4ccccc4-6)n3)c2)cc1 ; CC1(C)c2ccccc2-c2c1ccc1c2c2ccccc2n1-c1ccc(-c2nc(-c3ccccc3)nc(-c3cccc(-c4ccccc4)c3)n2)cc1 ; CC1(C)c2ccccc2-c2c1ccc1c2c2ccccc2n1-c1ccc(-c2nc(-c3ccccc3)cc(-c3cccc(-c4ccccc4)c3)n2)cc1]

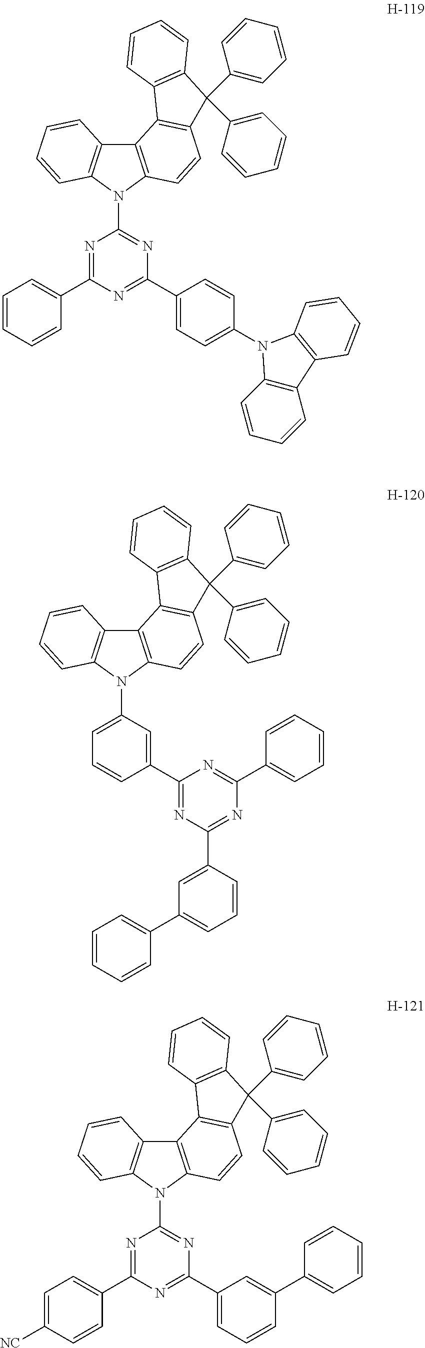 [SMILES: c1ccc(-c2nc(-c3ccc(-n4c5ccccc5c5ccccc54)cc3)nc(-n3c4ccccc4c4c5c(ccc43)C(c3ccccc3)(c3ccccc3)c3ccccc3-5)n2)cc1 ; c1ccc(-c2cccc(-c3nc(-c4ccccc4)nc(-c4cccc(-n5c6ccccc6c6c7c(ccc65)C(c5ccccc5)(c5ccccc5)c5ccccc5-7)c4)n3)c2)cc1 ; N#Cc1ccc(-c2nc(-c3cccc(-c4ccccc4)c3)nc(-n3c4ccccc4c4c5c(ccc43)C(c3ccccc3)(c3ccccc3)c3ccccc3-5)n2)cc1]

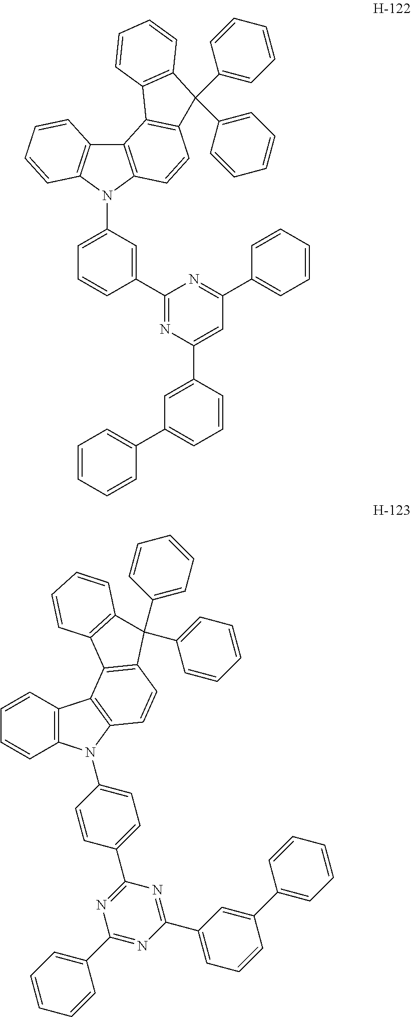 [SMILES: c1ccc(-c2cccc(-c3cc(-c4ccccc4)nc(-c4cccc(-n5c6ccccc6c6c7c(ccc65)C(c5ccccc5)(c5ccccc5)c5ccccc5-7)c4)n3)c2)cc1 ; c1ccc(-c2cccc(-c3nc(-c4ccccc4)nc(-c4ccc(-n5c6ccccc6c6c7c(ccc65)C(c5ccccc5)(c5ccccc5)c5ccccc5-7)cc4)n3)c2)cc1]

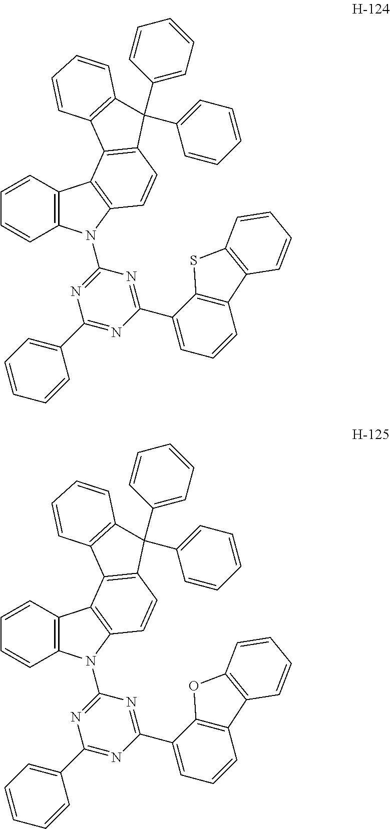 [SMILES: c1ccc(-c2nc(-c3cccc4c3sc3ccccc34)nc(-n3c4ccccc4c4c5c(ccc43)C(c3ccccc3)(c3ccccc3)c3ccccc3-5)n2)cc1 ; c1ccc(-c2nc(-c3cccc4c3oc3ccccc34)nc(-n3c4ccccc4c4c5c(ccc43)C(c3ccccc3)(c3ccccc3)c3ccccc3-5)n2)cc1]

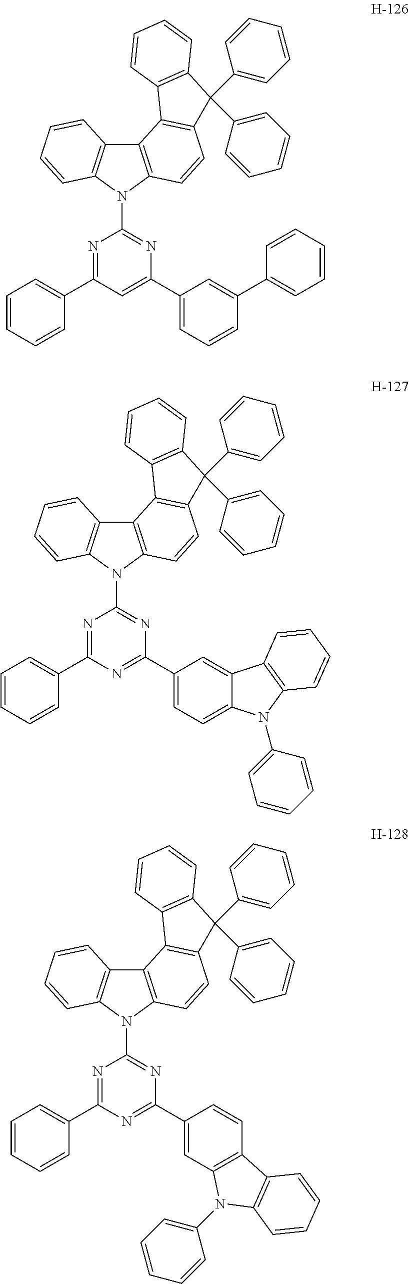

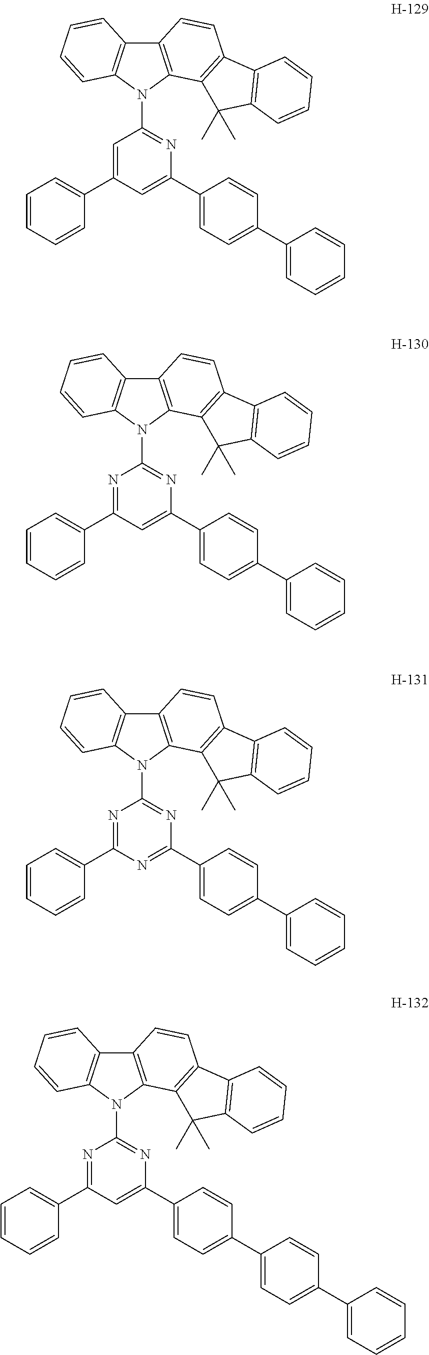 [SMILES: CC1(C)c2ccccc2-c2ccc3c4ccccc4n(-c4cc(-c5ccccc5)cc(-c5ccc(-c6ccccc6)cc5)n4)c3c21 ; CC1(C)c2ccccc2-c2ccc3c4ccccc4n(-c4nc(-c5ccccc5)cc(-c5ccc(-c6ccccc6)cc5)n4)c3c21 ; CC1(C)c2ccccc2-c2ccc3c4ccccc4n(-c4nc(-c5ccccc5)nc(-c5ccc(-c6ccccc6)cc5)n4)c3c21 ; CC1(C)c2ccccc2-c2ccc3c4ccccc4n(-c4nc(-c5ccccc5)cc(-c5ccc(-c6ccc(-c7ccccc7)cc6)cc5)n4)c3c21]

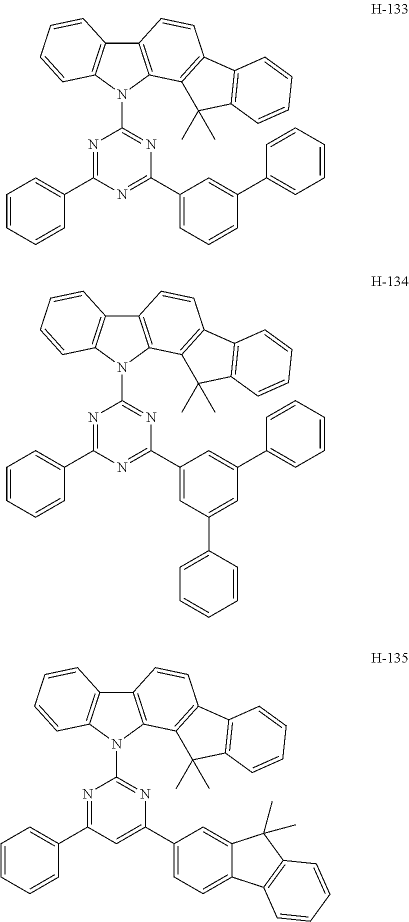 [SMILES: CC1(C)c2ccccc2-c2ccc3c4ccccc4n(-c4nc(-c5ccccc5)nc(-c5cccc(-c6ccccc6)c5)n4)c3c21 ; CC1(C)c2ccccc2-c2ccc3c4ccccc4n(-c4nc(-c5ccccc5)nc(-c5cc(-c6ccccc6)cc(-c6ccccc6)c5)n4)c3c21 ; CC1(C)c2ccccc2-c2ccc(-c3cc(-c4ccccc4)nc(-n4c5ccccc5c5ccc6c(c54)C(C)(C)c4ccccc4-6)n3)cc21]

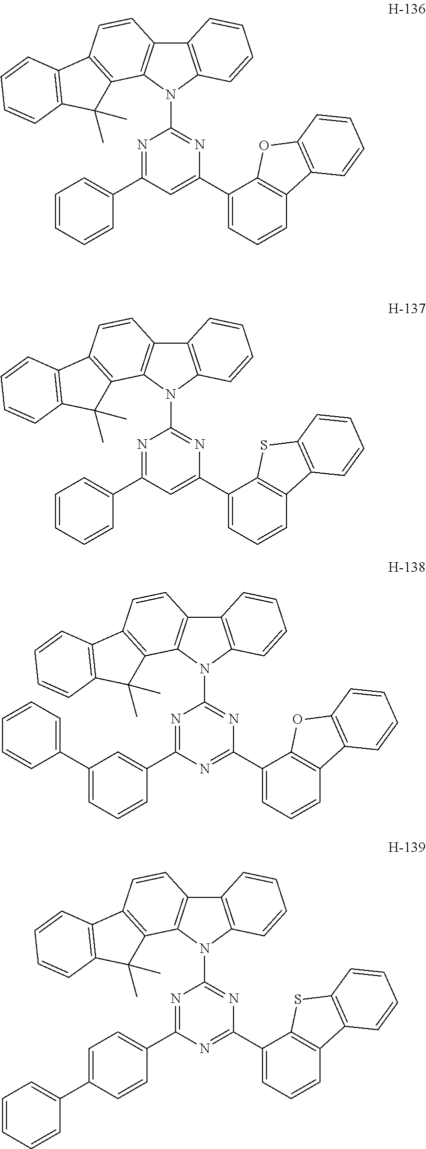 [SMILES: CC1(C)c2ccccc2-c2ccc3c4ccccc4n(-c4nc(-c5ccccc5)cc(-c5cccc6c5oc5ccccc56)n4)c3c21 ; CC1(C)c2ccccc2-c2ccc3c4ccccc4n(-c4nc(-c5ccccc5)cc(-c5cccc6c5sc5ccccc56)n4)c3c21 ; CC1(C)c2ccccc2-c2ccc3c4ccccc4n(-c4nc(-c5cccc(-c6ccccc6)c5)nc(-c5cccc6c5oc5ccccc56)n4)c3c21 ; CC1(C)c2ccccc2-c2ccc3c4ccccc4n(-c4nc(-c5ccc(-c6ccccc6)cc5)nc(-c5cccc6c5sc5ccccc56)n4)c3c21]

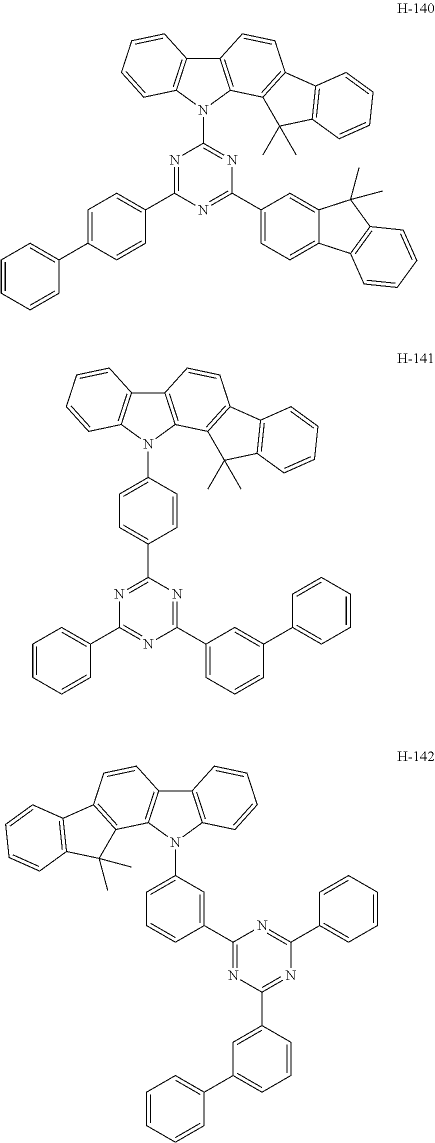 [SMILES: CC1(C)c2ccccc2-c2ccc(-c3nc(-c4ccc(-c5ccccc5)cc4)nc(-n4c5ccccc5c5ccc6c(c54)C(C)(C)c4ccccc4-6)n3)cc21 ; CC1(C)c2ccccc2-c2ccc3c4ccccc4n(-c4ccc(-c5nc(-c6ccccc6)nc(-c6cccc(-c7ccccc7)c6)n5)cc4)c3c21 ; CC1(C)c2ccccc2-c2ccc3c4ccccc4n(-c4cccc(-c5nc(-c6ccccc6)nc(-c6cccc(-c7ccccc7)c6)n5)c4)c3c21]

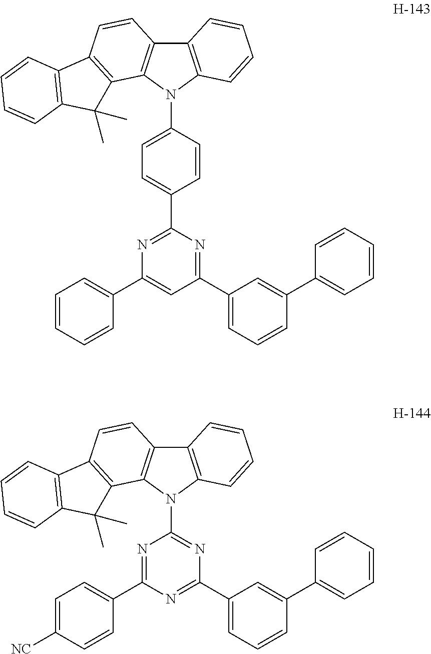 [SMILES: CC1(C)c2ccccc2-c2ccc3c4ccccc4n(-c4ccc(-c5nc(-c6ccccc6)cc(-c6cccc(-c7ccccc7)c6)n5)cc4)c3c21 ; CC1(C)c2ccccc2-c2ccc3c4ccccc4n(-c4nc(-c5ccc(C#N)cc5)nc(-c5cccc(-c6ccccc6)c5)n4)c3c21]

The organic electroluminescent compounds of the present invention can be prepared by a synthetic method known to a person skilled in the art. For example, they can be prepared according to the following reaction scheme.

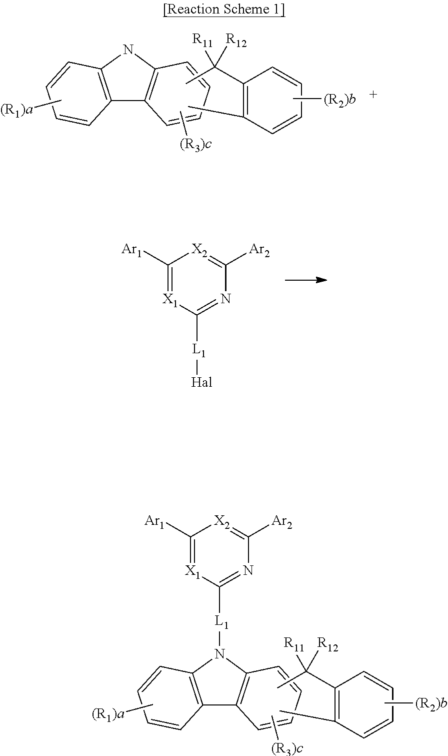

wherein X₁, X₂, L₁, Ar₁, Ar_{2,} R₁ to R_{3,} R₁₁, R₁₂, a, b, and c are as defined in formula 1, and Hal represents a halogen.

The present invention provides an organic electroluminescent material comprising the organic electroluminescent compound of formula 1, and an organic electroluminescent device comprising the material.

The above material can be comprised of the organic electroluminescent compound according to the present invention alone, or can further include conventional materials generally used in organic electroluminescent materials.

The organic electroluminescent device comprises a first electrode; a second electrode; and at least one organic layer between the first and second electrodes. The organic layer may comprise at least one organic electroluminescent compound of formula 1.

One of the first and second electrodes can be an anode, and the other can be a cathode. The organic layer comprises a light-emitting layer, and may further comprise at least one layer selected from the group consisting of a hole injection layer, a hole transport layer, an electron transport layer, an electron injection layer, an interlayer, a hole blocking layer, and an electron blocking layer.

The compound of formula 1 according to the present invention can be comprised in the light-emitting layer. Where used in the light-emitting layer, the compound of formula 1 according to the present invention can be comprised as a phosphorescent host material. Preferably, the light-emitting layer can further comprise one or more dopants. If necessary, a compound other than the compound of formula 1 according to the present invention can be additionally comprised as a second host material. Herein, the weight ratio of the first host material to the second host material is in the range of 1:99 to 99:1.

The second host material can be any of the known phosphorescent hosts. Specifically, the phosphorescent host selected from the group consisting of the compounds of formulae 11 to 15 below is preferable in terms of luminous efficiency.

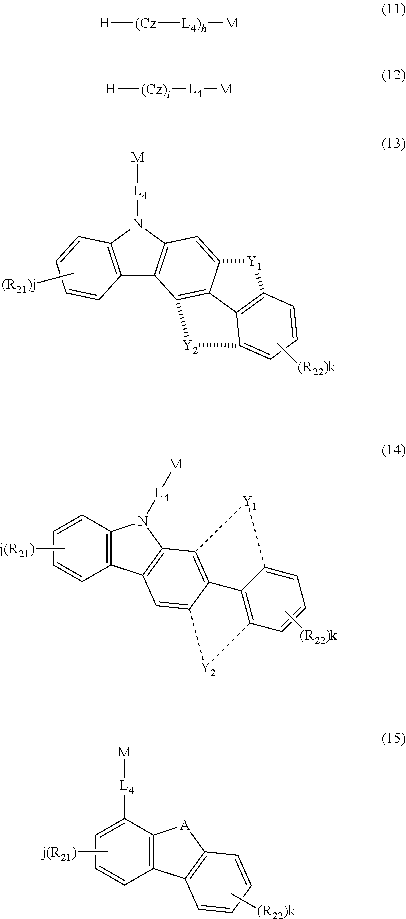

wherein Cz represents the following structure;

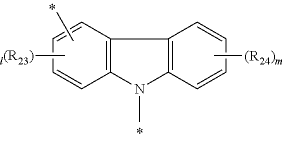

A represents —O— or —S—;

R₂₁ to R₂₄ each independently represent hydrogen, deuterium, a halogen, a substituted or unsubstituted (C1-C30)alkyl, a substituted of unsubstituted (C6-C30)aryl, a substituted or unsubstituted 5- to 30-membered heteroaryl, or —SiR₂₅R₂₆R₂₇;

R₂₅ to R₂₇ each independently represent a substituted or unsubstituted (C1-C30)alkyl, or a substituted or unsubstituted (C6-C30)aryl;

L₄ represents a single bond, a substituted or unsubstituted (C6-C30)arylene, or a substituted or unsubstituted 5- to 30-membered heteroarylene;

M represents a substituted or unsubstituted (C6-C30)aryl, or a substituted or unsubstituted 5- to 30-membered heteroaryl;

Y₁ and Y₂ each independently represent —O—, —S—, —N(R₃₁)—, or —C(R₃₂)(R₃₃)—, provided that Y₁ and Y₂ do not simultaneously exist;

R₃₁ to R₃₃ each independently represent a substituted or unsubstituted (C1-C30)alkyl, a substituted or unsubstituted (C6-C30)aryl, or a substituted or unsubstituted 5- to 30-membered heteroaryl, and R₃₂ and R₃₃ may be the same or different;

h and i each independently represent an integer of 1 to 3;

j, k, l, and m each independently represent an integer of 0 to 4; and

where h, i, j, k, l, or m is an integer of 2 or more, each of (Cz-L₄), each of (Cz), each of R₂₁, each of R₂₂, each of R₂₃, or each of R₂₄ may be the same or different.

Specifically, preferable examples of the second host material are as follows:

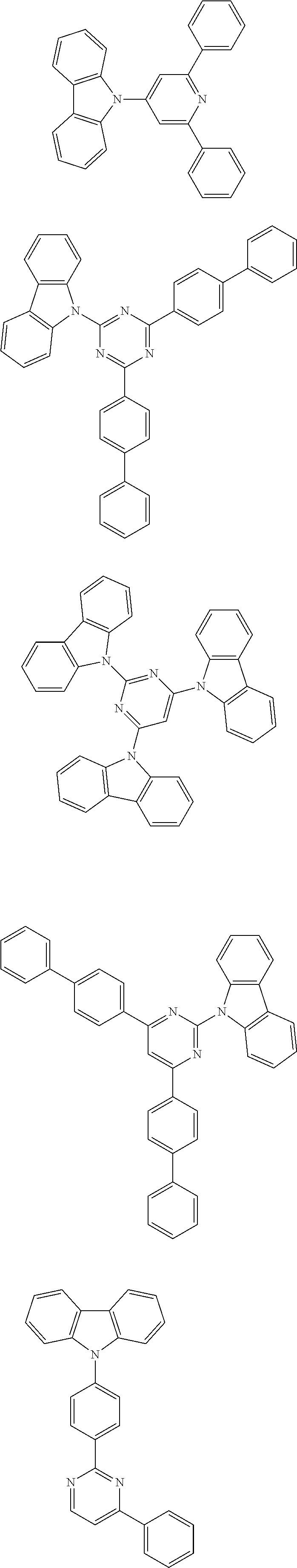 [SMILES: c1ccc(-c2cc(-n3c4ccccc4c4ccccc43)cc(-c3ccccc3)n2)cc1 ; c1ccc(-c2ccc(-c3nc(-c4ccc(-c5ccccc5)cc4)nc(-n4c5ccccc5c5ccccc54)n3)cc2)cc1 ; c1ccc2c(c1)c1ccccc1n2-c1cc(-n2c3ccccc3c3ccccc32)nc(-n2c3ccccc3c3ccccc32)n1 ; c1ccc(-c2ccc(-c3cc(-c4ccc(-c5ccccc5)cc4)nc(-n4c5ccccc5c5ccccc54)n3)cc2)cc1 ; c1ccc(-c2ccnc(-c3ccc(-n4c5ccccc5c5ccccc54)cc3)n2)cc1]

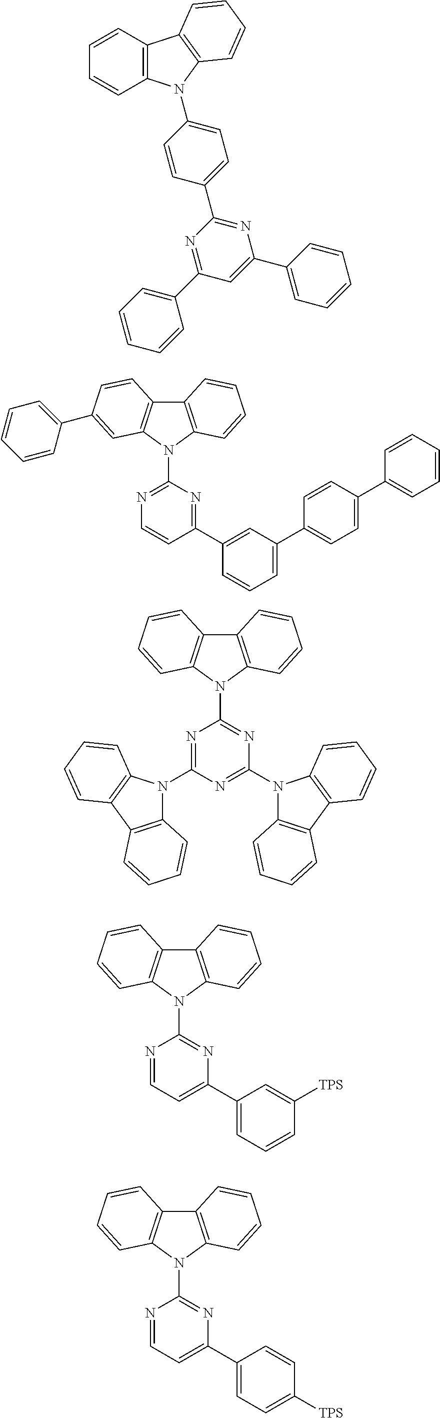

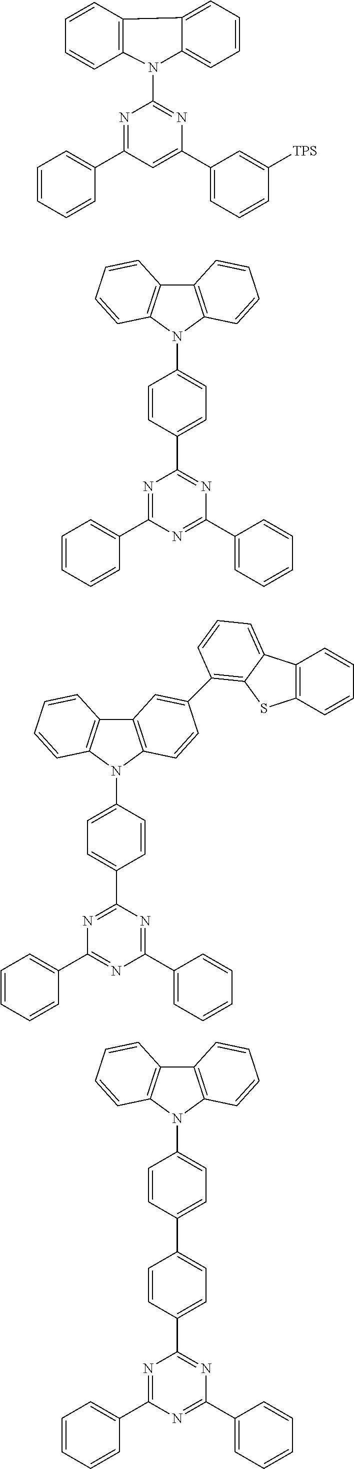

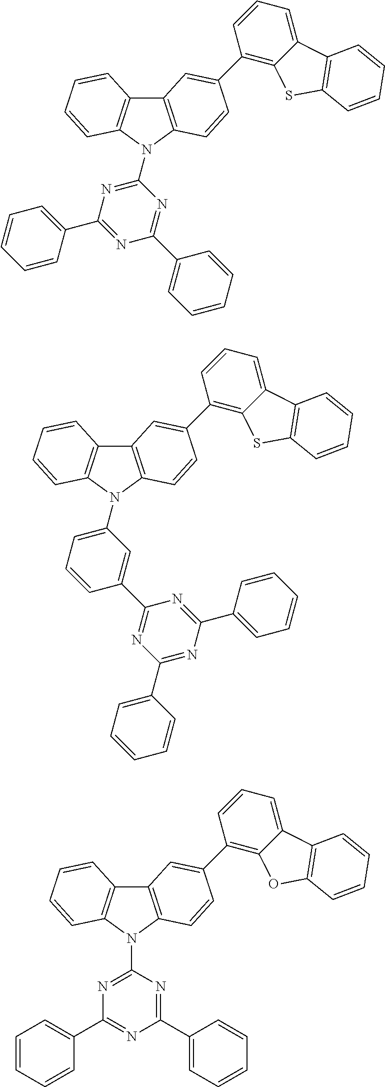

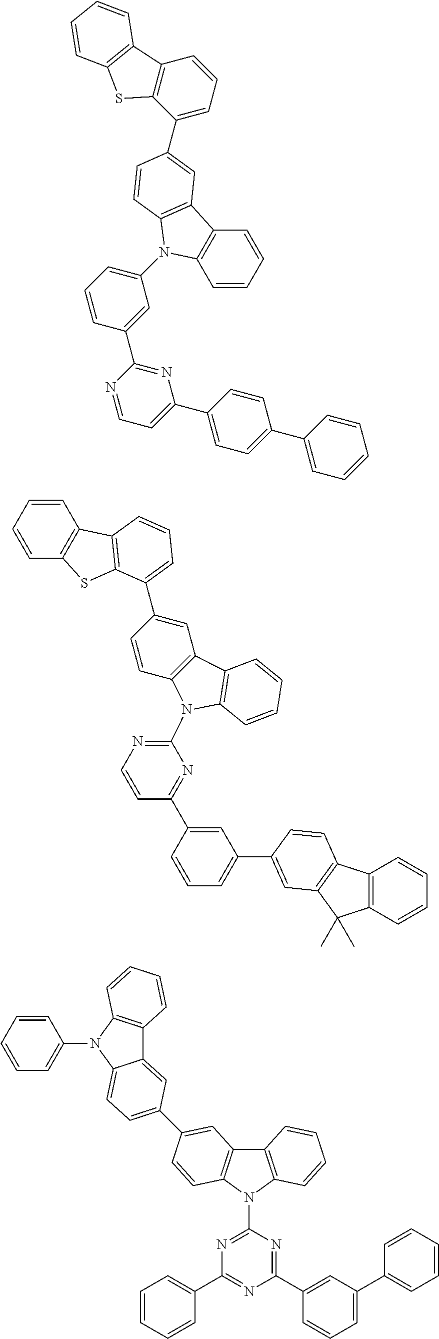 [SMILES: c1ccc(-c2ccc(-c3ccnc(-c4cccc(-n5c6ccccc6c6cc(-c7cccc8c7sc7ccccc78)ccc65)c4)n3)cc2)cc1 ; CC1(C)c2ccccc2-c2ccc(-c3cccc(-c4ccnc(-n5c6ccccc6c6cc(-c7cccc8c7sc7ccccc78)ccc65)n4)c3)cc21 ; c1ccc(-c2cccc(-c3nc(-c4ccccc4)nc(-n4c5ccccc5c5cc(-c6ccc7c(c6)c6ccccc6n7-c6ccccc6)ccc54)n3)c2)cc1]

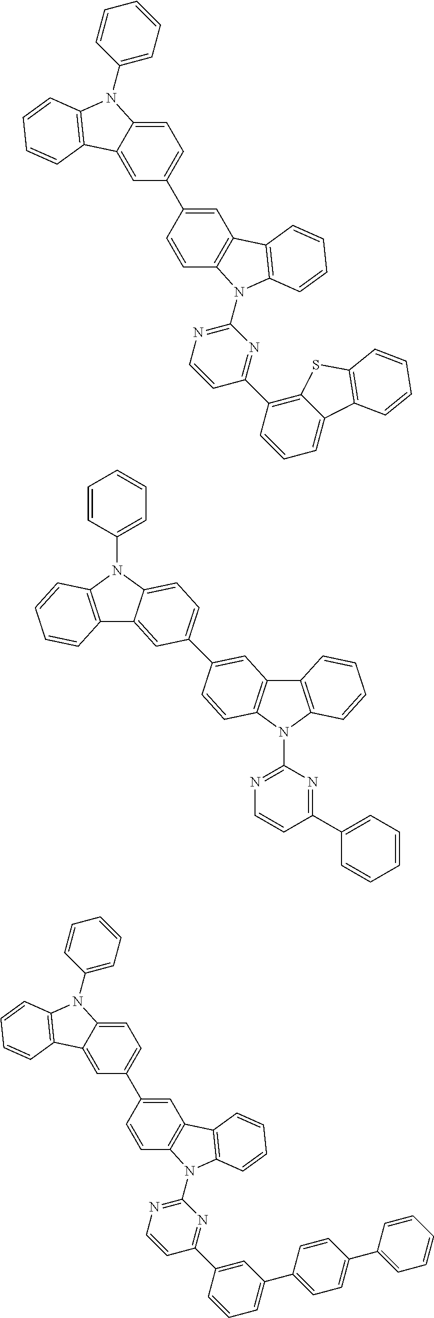 [SMILES: c1ccc(-n2c3ccccc3c3cc(-c4ccc5c(c4)c4ccccc4n5-c4nccc(-c5cccc6c5sc5ccccc56)n4)ccc32)cc1 ; c1ccc(-c2ccnc(-n3c4ccccc4c4cc(-c5ccc6c(c5)c5ccccc5n6-c5ccccc5)ccc43)n2)cc1 ; c1ccc(-c2ccc(-c3cccc(-c4ccnc(-n5c6ccccc6c6cc(-c7ccc8c(c7)c7ccccc7n8-c7ccccc7)ccc65)n4)c3)cc2)cc1]

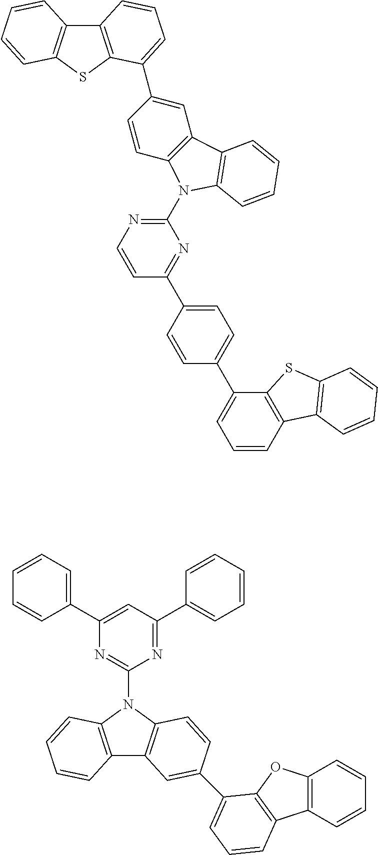 [SMILES: c1ccc2c(c1)sc1c(-c3ccc(-c4ccnc(-n5c6ccccc6c6cc(-c7cccc8c7sc7ccccc78)ccc65)n4)cc3)cccc12 ; c1ccc(-c2cc(-c3ccccc3)nc(-n3c4ccccc4c4cc(-c5cccc6c5oc5ccccc56)ccc43)n2)cc1]

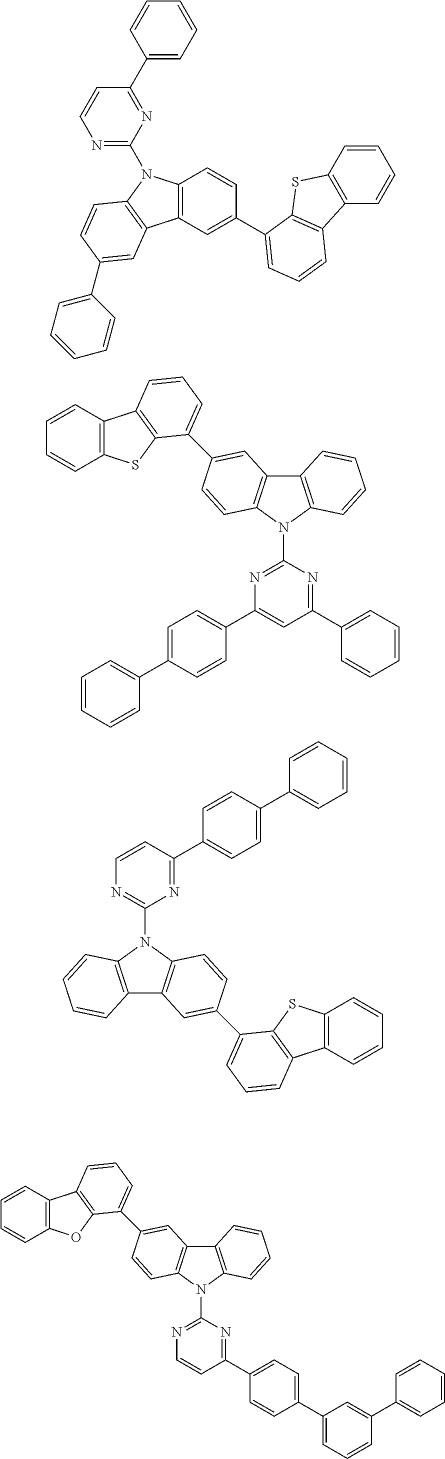

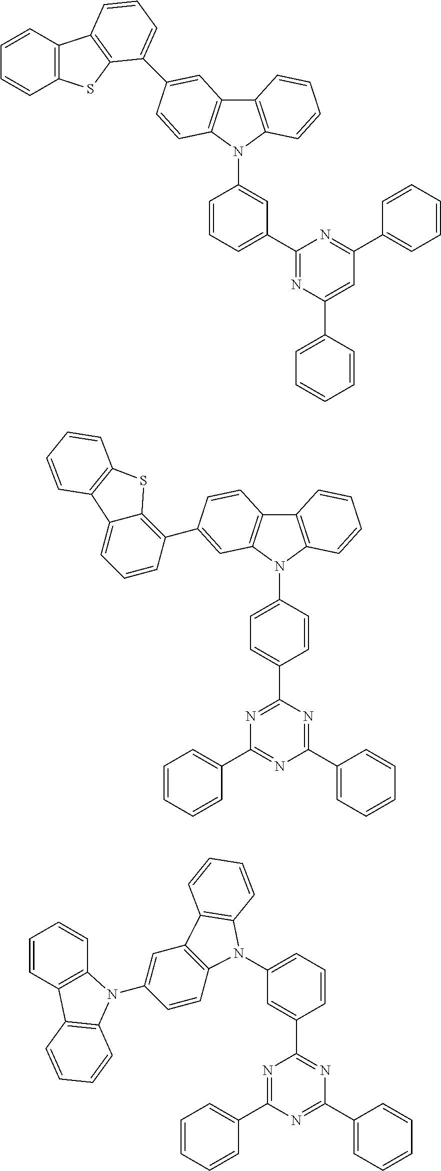

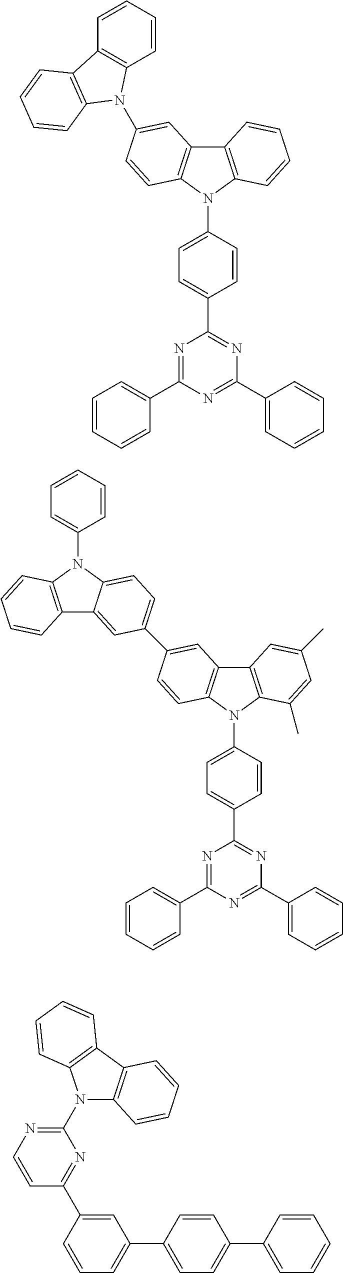

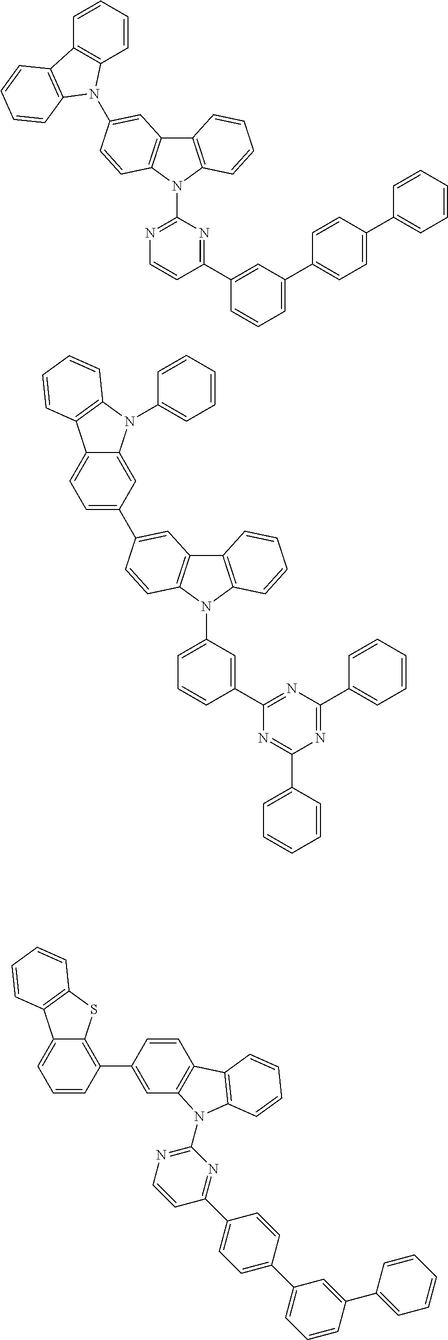 [SMILES: c1ccc(-c2ccc(-c3cccc(-c4ccnc(-n5c6ccccc6c6cc(-n7c8ccccc8c8ccccc87)ccc65)n4)c3)cc2)cc1 ; c1ccc(-c2nc(-c3ccccc3)nc(-c3cccc(-n4c5ccccc5c5cc(-c6ccc7c8ccccc8n(-c8ccccc8)c7c6)ccc54)c3)n2)cc1 ; c1ccc(-c2cccc(-c3ccc(-c4ccnc(-n5c6ccccc6c6ccc(-c7cccc8c7sc7ccccc78)cc65)n4)cc3)c2)cc1]

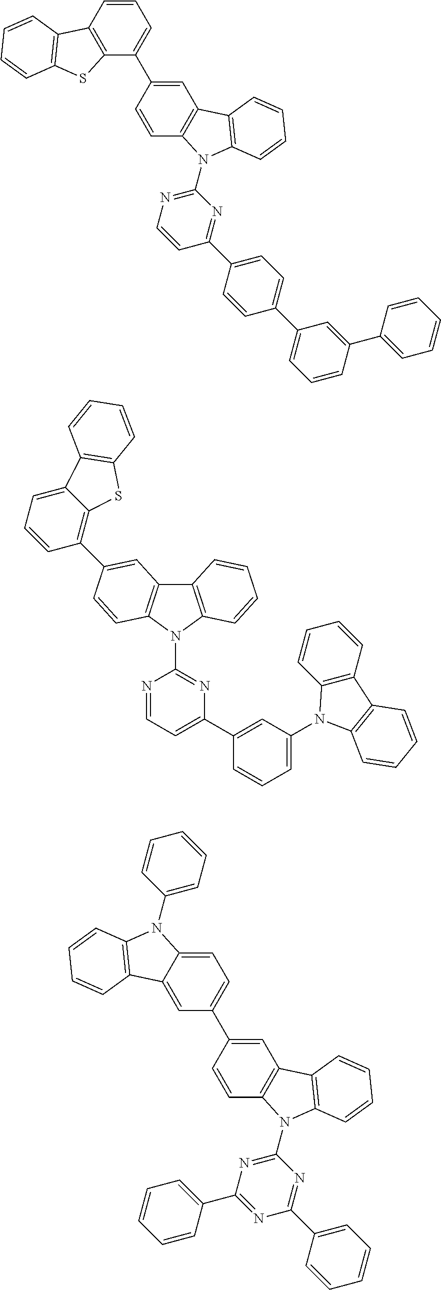 [SMILES: c1ccc(-c2cccc(-c3ccc(-c4ccnc(-n5c6ccccc6c6cc(-c7cccc8c7sc7ccccc78)ccc65)n4)cc3)c2)cc1 ; c1cc(-c2ccnc(-n3c4ccccc4c4cc(-c5cccc6c5sc5ccccc56)ccc43)n2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

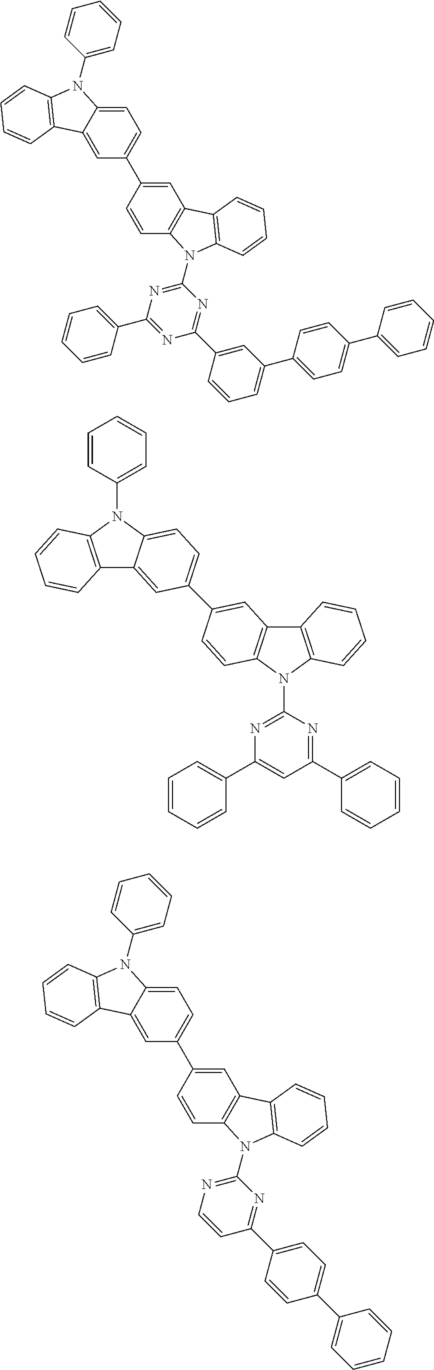 [SMILES: c1ccc(-c2ccc(-c3cccc(-c4nc(-c5ccccc5)nc(-n5c6ccccc6c6cc(-c7ccc8c(c7)c7ccccc7n8-c7ccccc7)ccc65)n4)c3)cc2)cc1 ; c1ccc(-c2cc(-c3ccccc3)nc(-n3c4ccccc4c4cc(-c5ccc6c(c5)c5ccccc5n6-c5ccccc5)ccc43)n2)cc1 ; c1ccc(-c2ccc(-c3ccnc(-n4c5ccccc5c5cc(-c6ccc7c(c6)c6ccccc6n7-c6ccccc6)ccc54)n3)cc2)cc1]

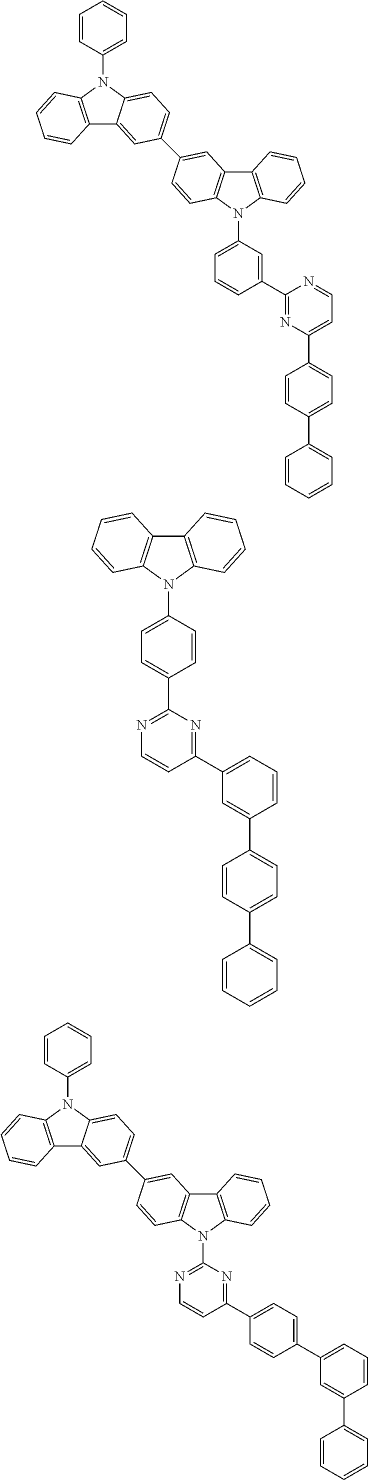 [SMILES: c1ccc(-c2ccc(-c3ccnc(-c4cccc(-n5c6ccccc6c6cc(-c7ccc8c(c7)c7ccccc7n8-c7ccccc7)ccc65)c4)n3)cc2)cc1 ; c1ccc(-c2ccc(-c3cccc(-c4ccnc(-c5ccc(-n6c7ccccc7c7ccccc76)cc5)n4)c3)cc2)cc1 ; c1ccc(-c2cccc(-c3ccc(-c4ccnc(-n5c6ccccc6c6cc(-c7ccc8c(c7)c7ccccc7n8-c7ccccc7)ccc65)n4)cc3)c2)cc1]

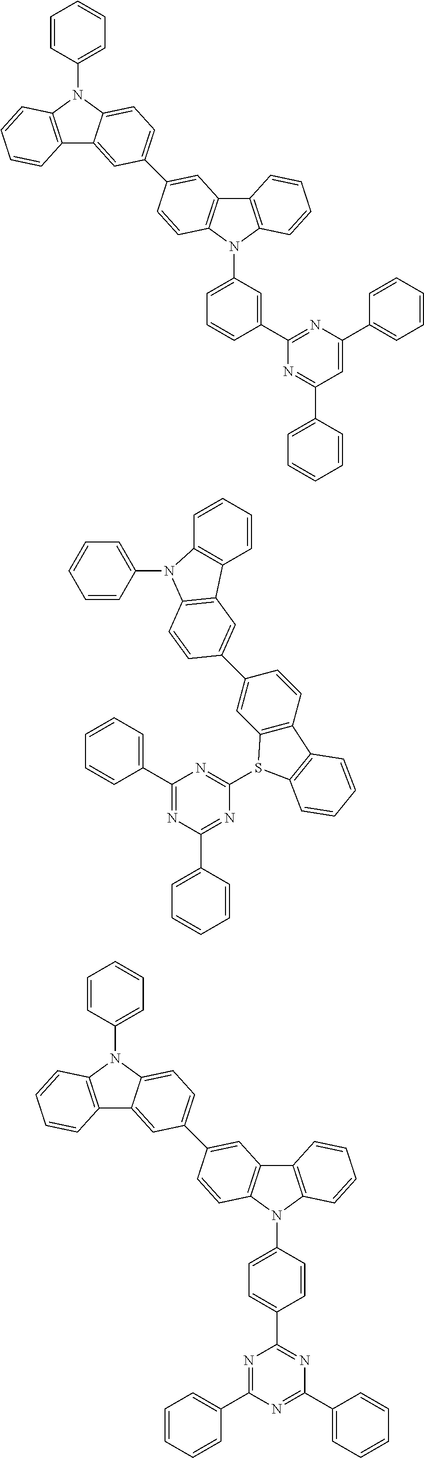

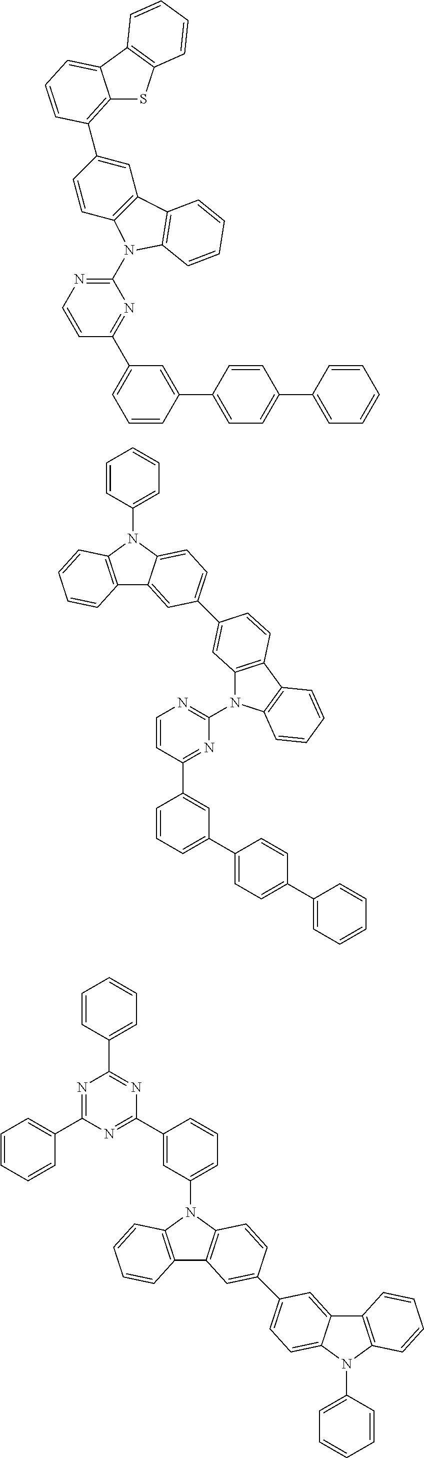

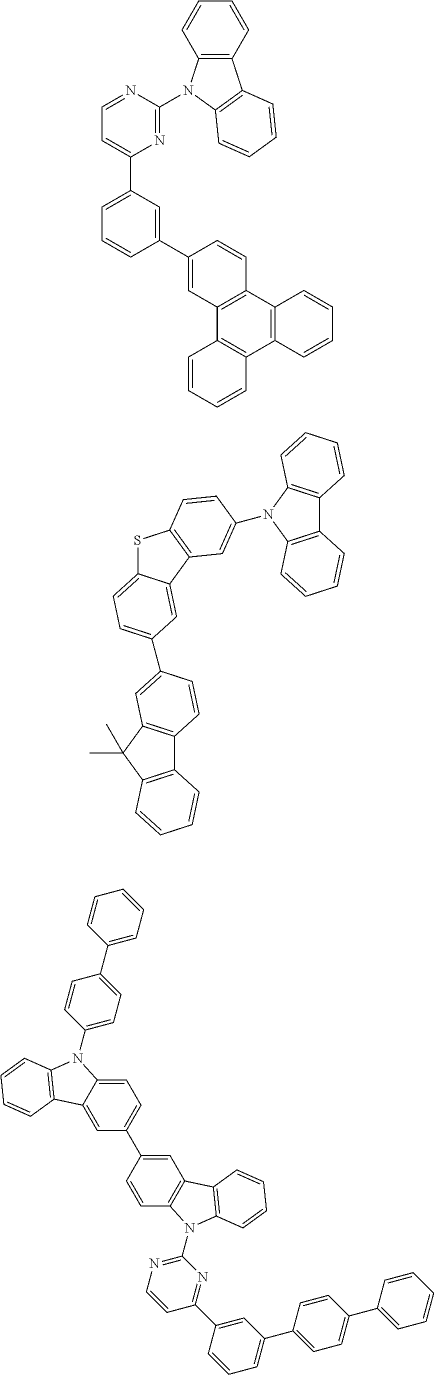 [SMILES: c1cc(-c2ccc3c4ccccc4c4ccccc4c3c2)cc(-c2ccnc(-n3c4ccccc4c4ccccc43)n2)c1 ; CC1(C)c2ccccc2-c2ccc(-c3ccc4sc5ccc(-n6c7ccccc7c7ccccc76)cc5c4c3)cc21 ; c1ccc(-c2ccc(-c3cccc(-c4ccnc(-n5c6ccccc6c6cc(-c7ccc8c(c7)c7ccccc7n8-c7ccc(-c8ccccc8)cc7)ccc65)n4)c3)cc2)cc1]

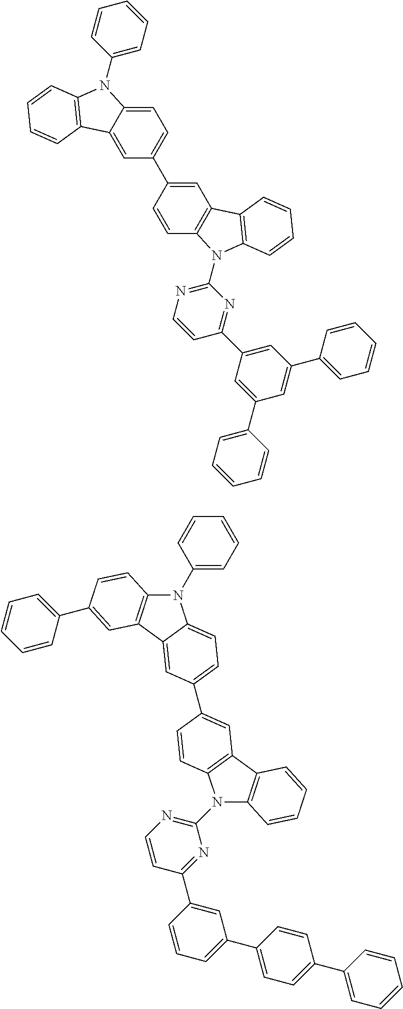

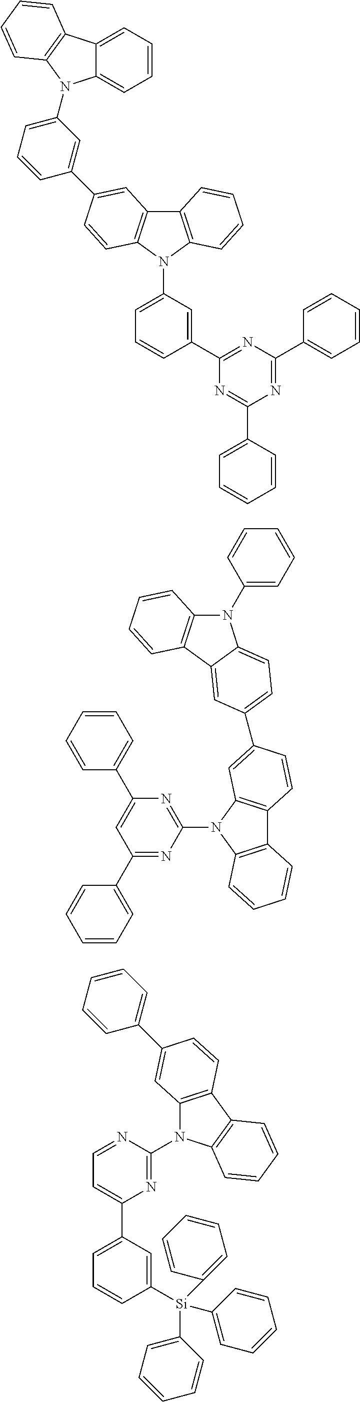

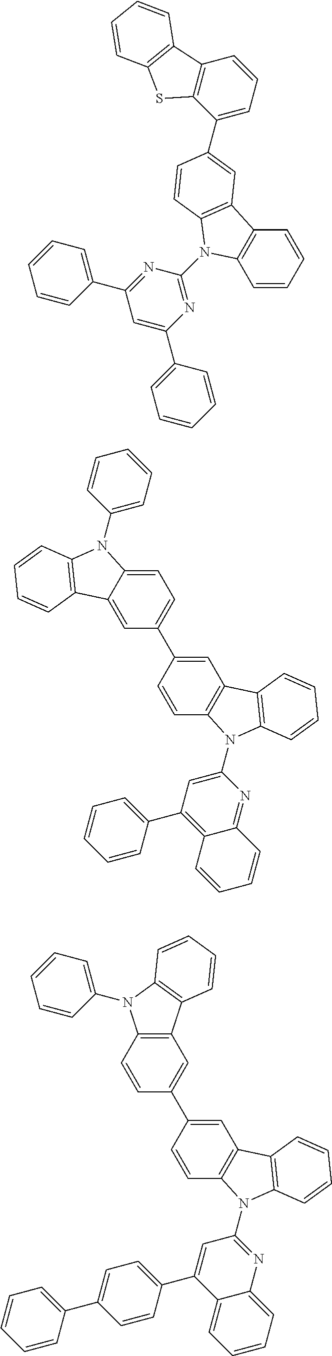

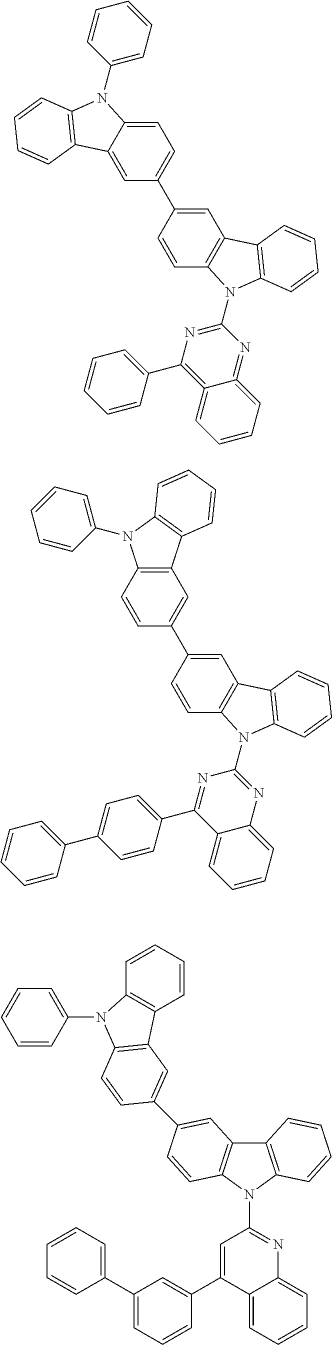

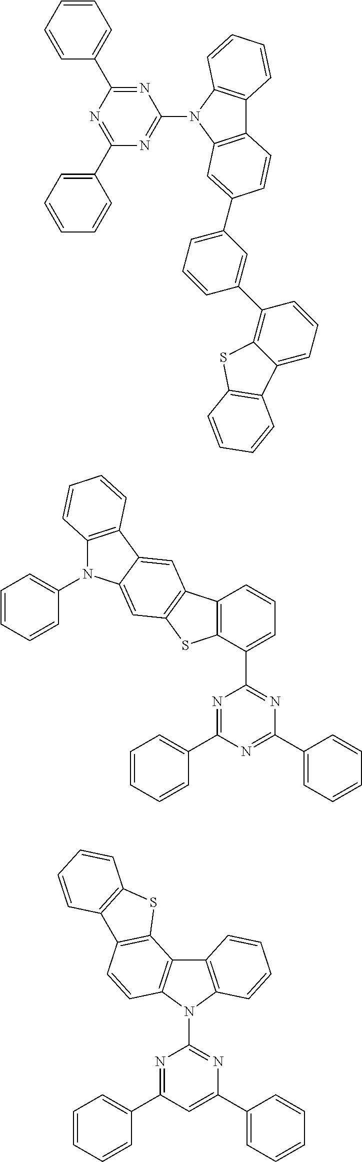 [SMILES: c1ccc(-c2nc(-c3ccccc3)nc(-n3c4ccccc4c4ccc(-c5cccc(-c6cccc7c6sc6ccccc67)c5)cc43)n2)cc1 ; c1ccc(-c2nc(-c3ccccc3)nc(-c3cccc4c3sc3cc5c(cc34)c3ccccc3n5-c3ccccc3)n2)cc1 ; c1ccc(-c2cc(-c3ccccc3)nc(-n3c4ccccc4c4c5sc6ccccc6c5ccc43)n2)cc1]

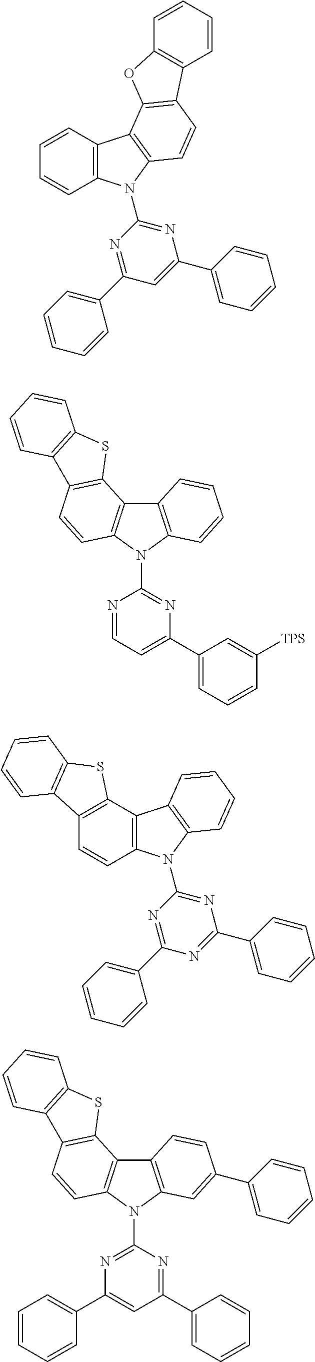

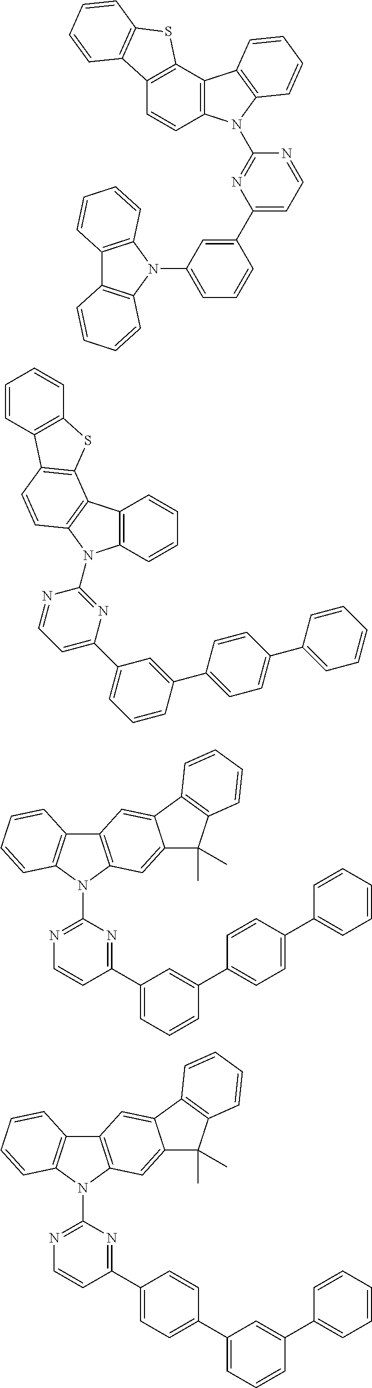

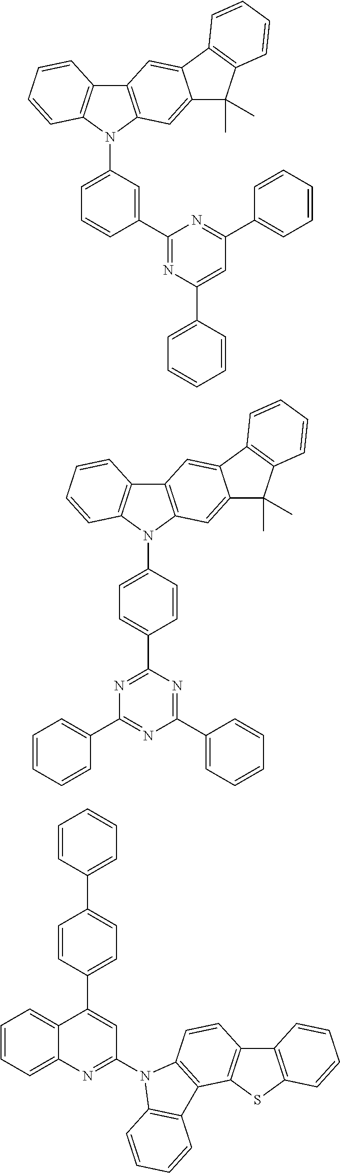

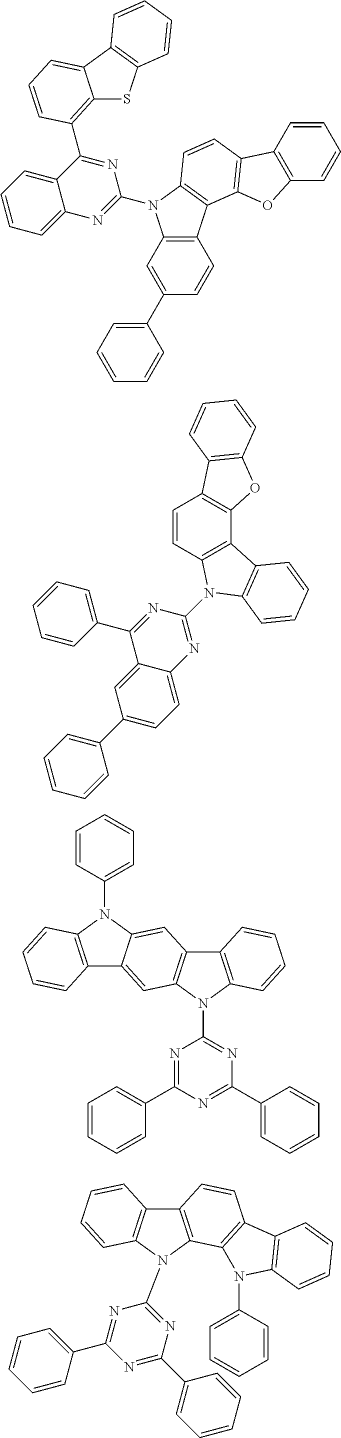

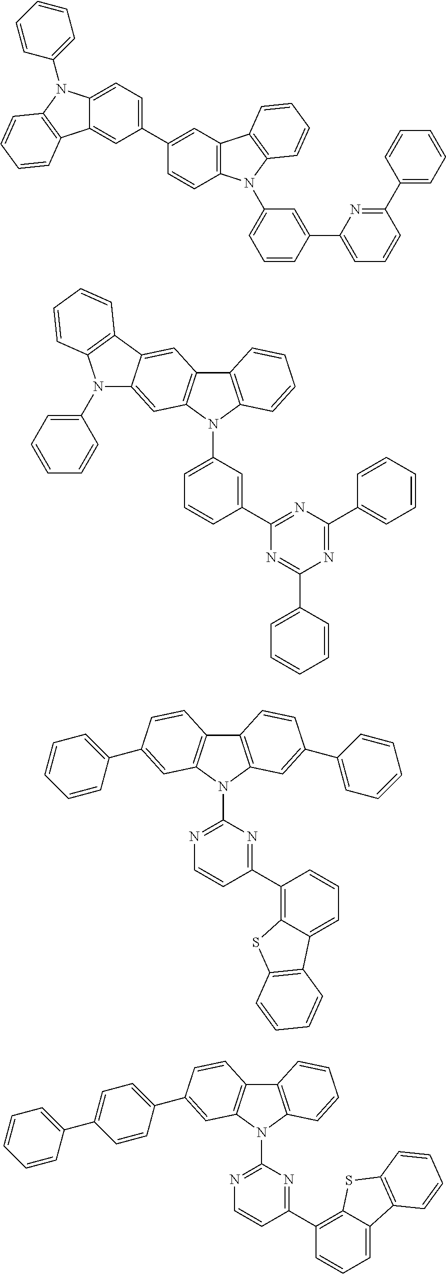

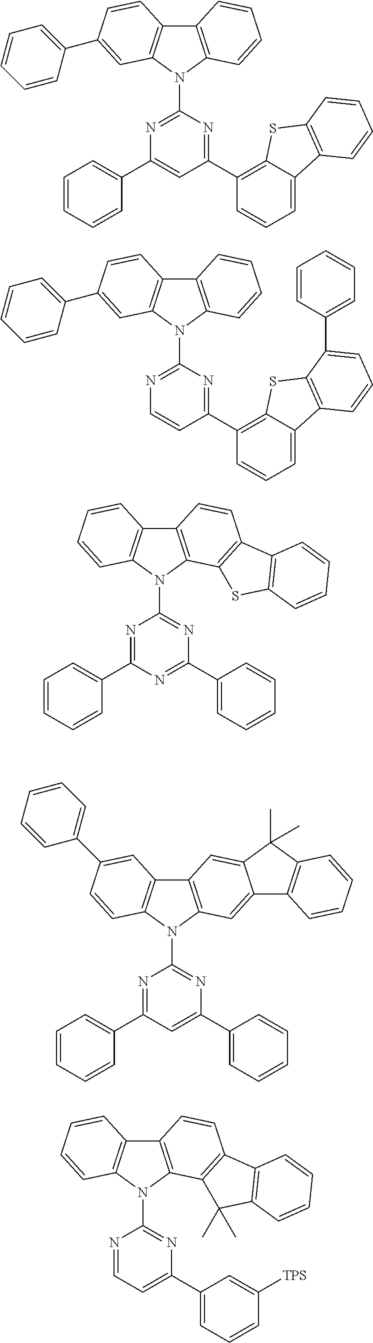

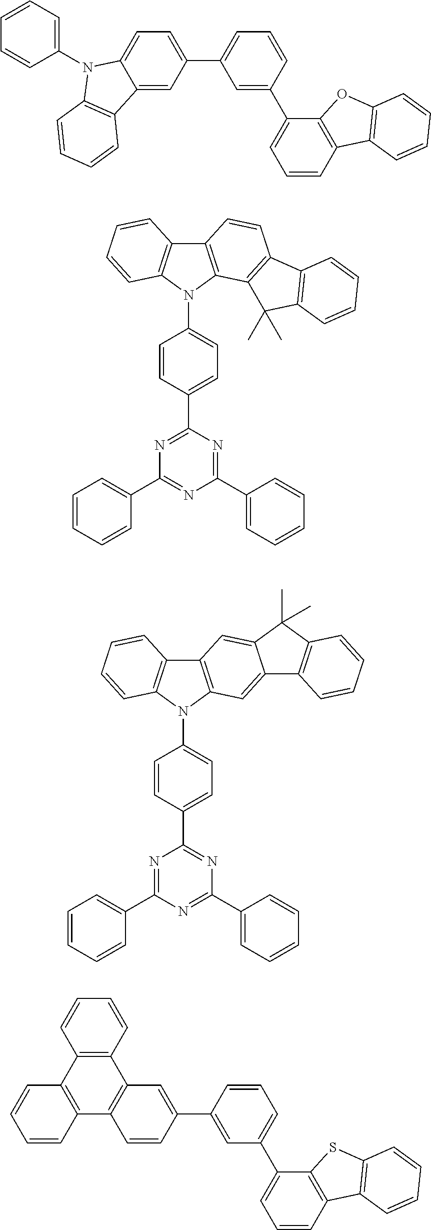

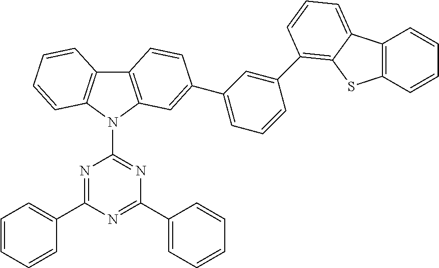 [SMILES: c1ccc(-c2nc(-c3ccccc3)nc(-n3c4ccccc4c4ccc(-c5cccc(-c6cccc7c6sc6ccccc67)c5)cc43)n2)cc1]

[wherein TPS represents a triphenylsilyl group]

The dopant comprised in the organic electroluminescent device according to the present invention is preferably at least one phosphorescent dopant. The dopant materials applied to the organic electroluminescent device according to the present invention are not limited, but may be preferably selected from metallated complex compounds of iridium, osmium, copper, and platinum, more preferably selected from ortho-metallated complex compounds of iridium, osmium, copper, and platinum, and even more preferably ortho-metallated iridium complex compounds.

The dopants comprised in the organic electroluminescent device of the present invention may be preferably selected from compounds represented by the following formulae 101 to 103.

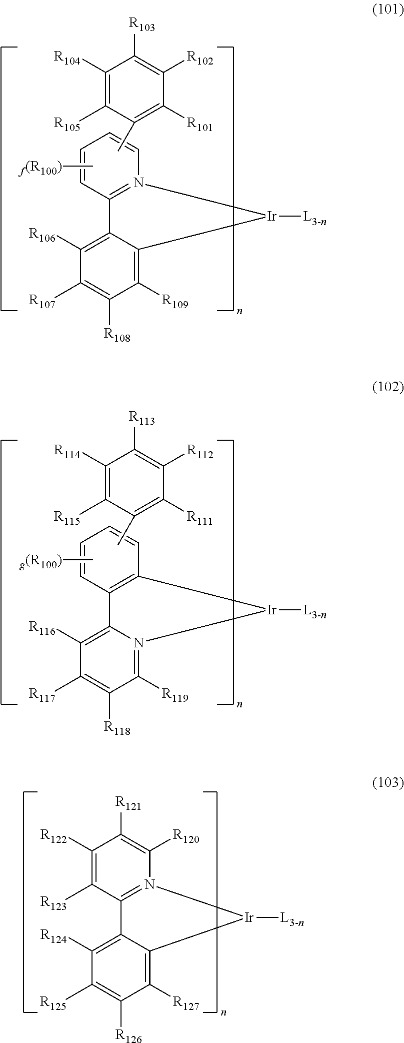

wherein L is selected from the following structures:

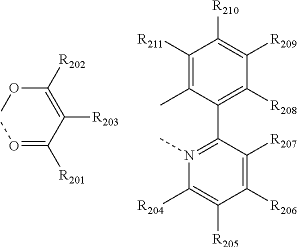

R₁₀₀ represents hydrogen, a substituted or unsubstituted (C1-C30)alkyl, or a substituted or unsubstituted (C3-C30)cycloalkyl;

R₁₀₁ to R₁₀₉, and R₁₁₁ to R₁₂₃ each independently represent hydrogen, deuterium, a halogen, a (C1-C30)alkyl unsubstituted or substituted with a halogen(s), a substituted or unsubstituted (C3-C30)cycloalkyl, a substituted or unsubstituted (C6-C30)aryl, a cyano, or a substituted or unsubstituted (C1-C30)alkoxy; adjacent substituents of R₁₀₆ to R₁₀₉ may be linked to each other to form a substituted or unsubstituted fused ring, e.g., fluorene unsubstituted or substituted with alkyl, dibenzothiophene unsubstituted or substituted with alkyl, or dibenzofuran unsubstituted or substituted with alkyl; and adjacent substituents of R₁₂₀ to R₁₂₃ may be linked to each other to form a substituted or unsubstituted fused ring, e.g., quinoline unsubstituted or substituted with alkyl or aryl;

R₁₂₄ to R₁₂₇ each independently represent hydrogen, deuterium, a halogen, a substituted or unsubstituted (C1-C30)alkyl, or a substituted or unsubstituted (C6-C30)aryl; and adjacent substituents of R₁₂₄ to R₁₂₇ may be linked to each other to form a substituted or unsubstituted fused ring, e.g., fluorene unsubstituted or substituted with alkyl, dibenzothiophene unsubstituted or substituted with alkyl, or dibenzofuran unsubstituted or substituted with alkyl;

R₂₀₁ to R₂₁₁ each independently represent hydrogen, deuterium, a halogen, a (C1-C30)alkyl unsubstituted or substituted with a halogen(s), a substituted or unsubstituted (C3-C30)cycloalkyl, or a substituted or unsubstituted (C6-C30)aryl, and adjacent substituents of R₂₀₈ to R₂₁₁ may be linked to each other to form a substituted or unsubstituted fused ring, e.g., fluorene unsubstituted or substituted with alkyl, dibenzothiophene unsubstituted or substituted with alkyl, or dibenzofuran unsubstituted or substituted with alkyl;

f and g each independently represent an integer of 1 to 3; where f or g is an integer of 2 or more, each of R₁₀₀ may be the same or different; and

n represents an integer of 1 to 3.

Specifically, the dopant compounds include the following:

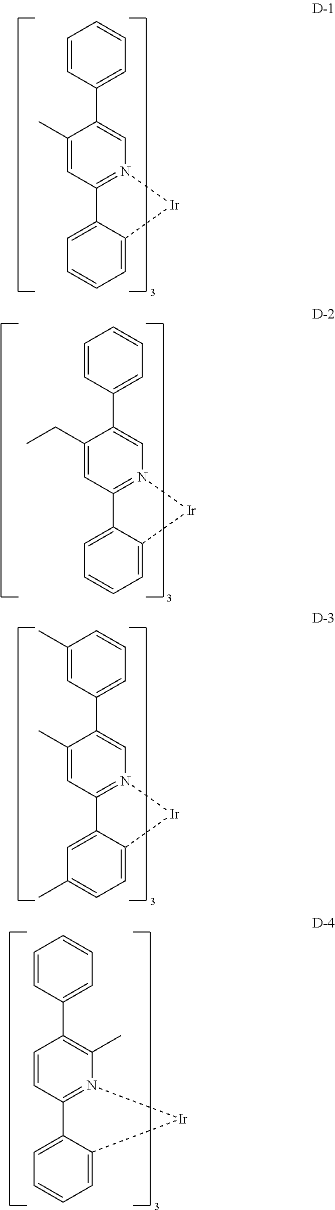

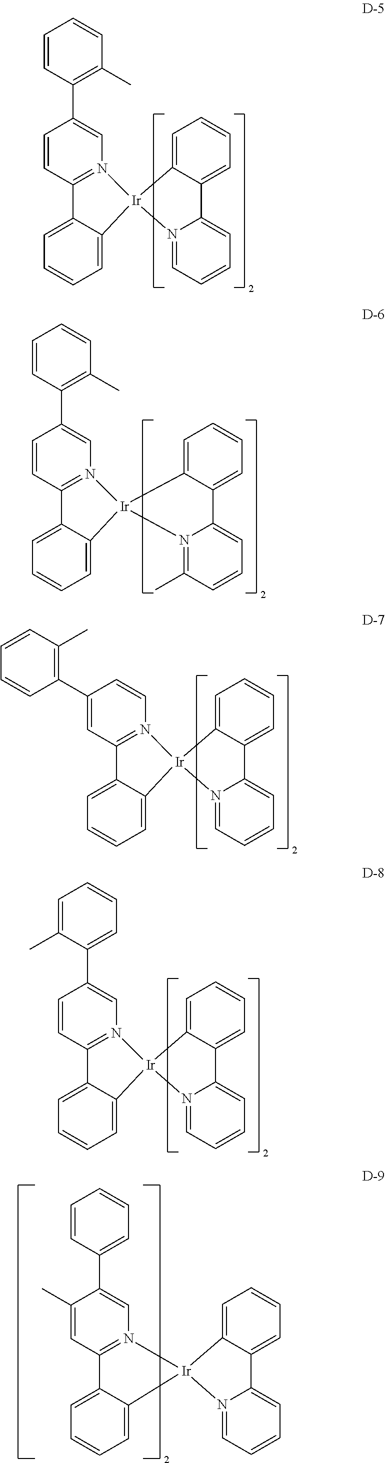

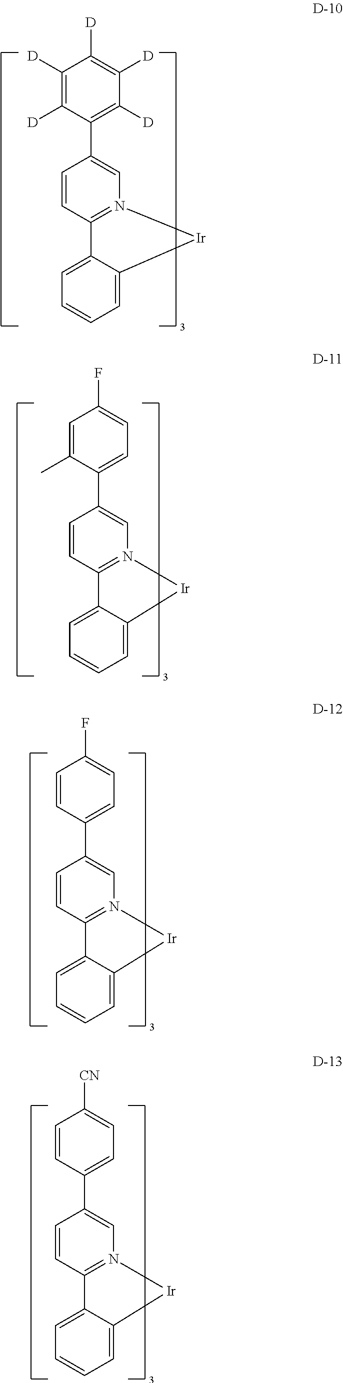

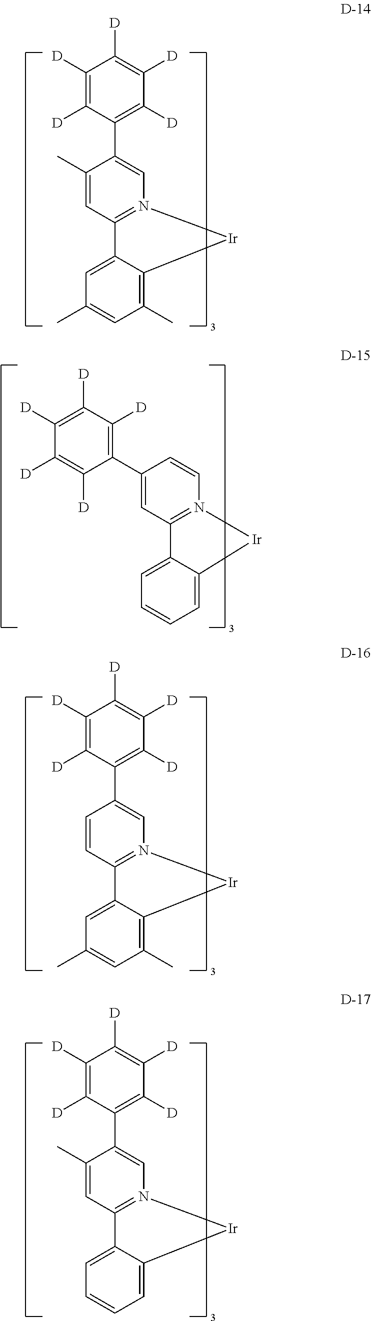

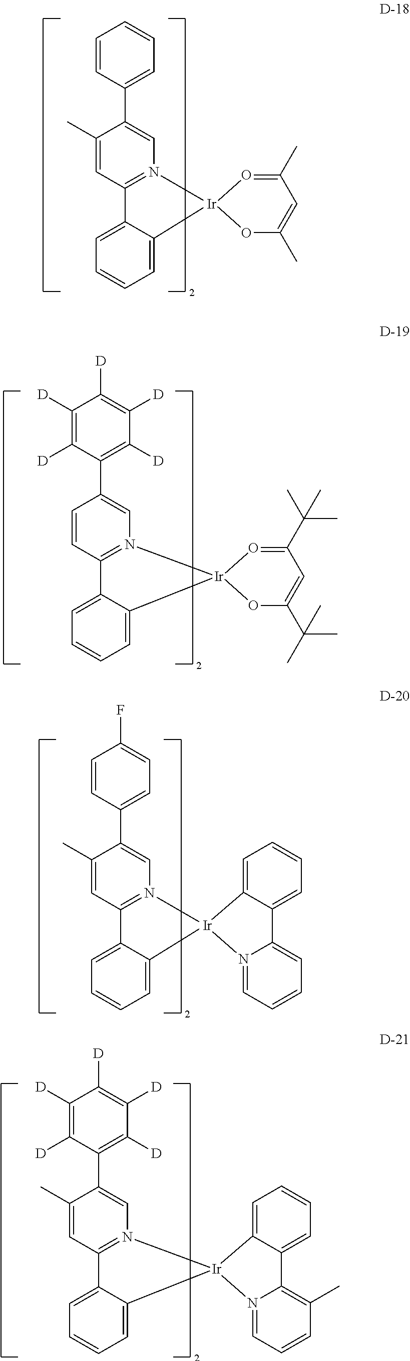

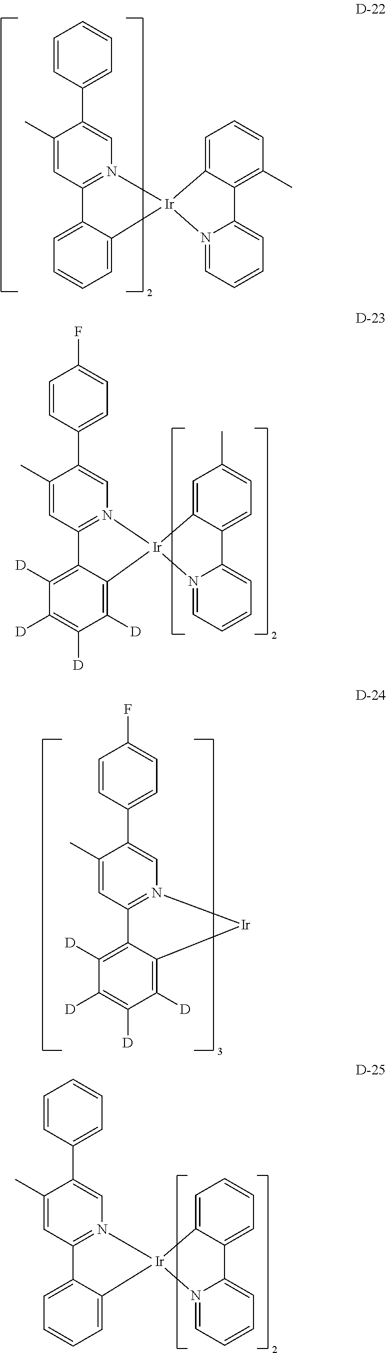

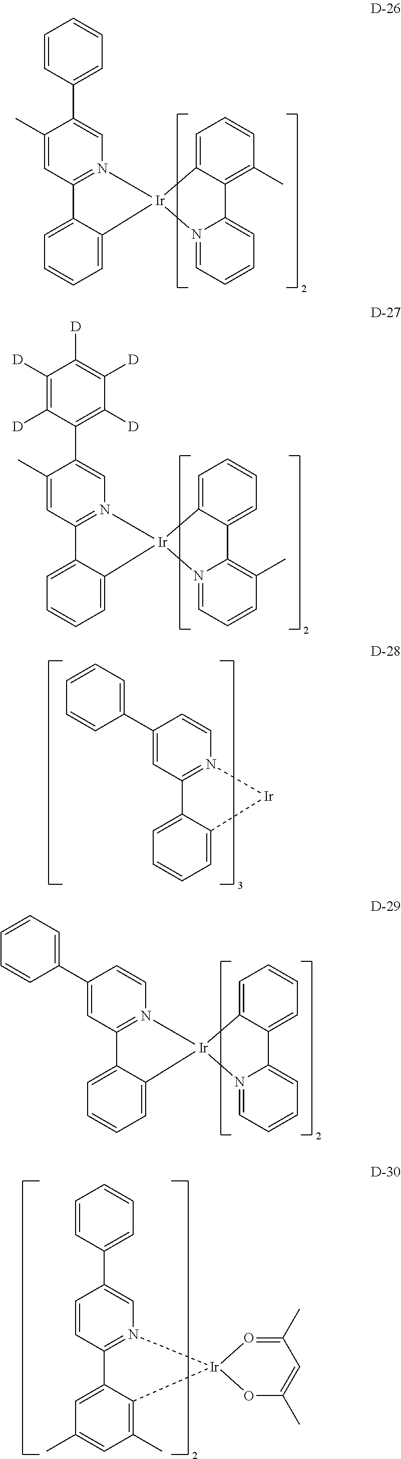

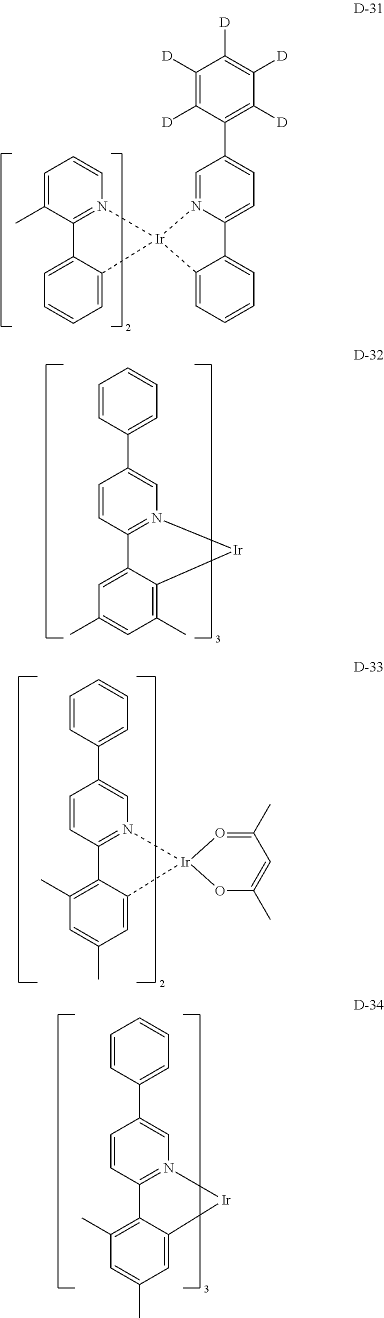

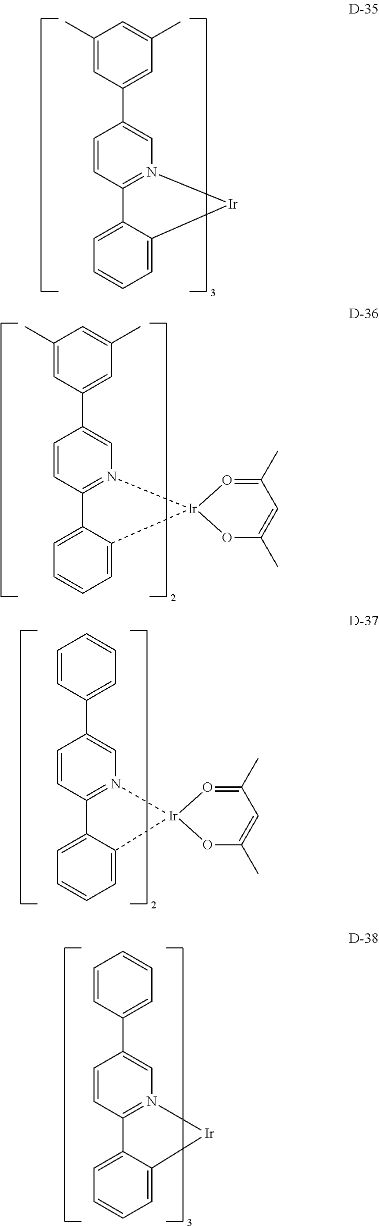

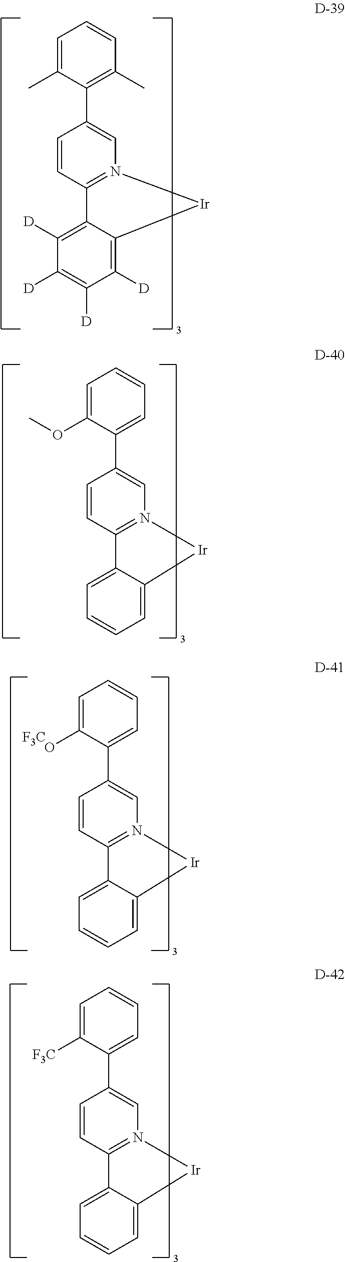

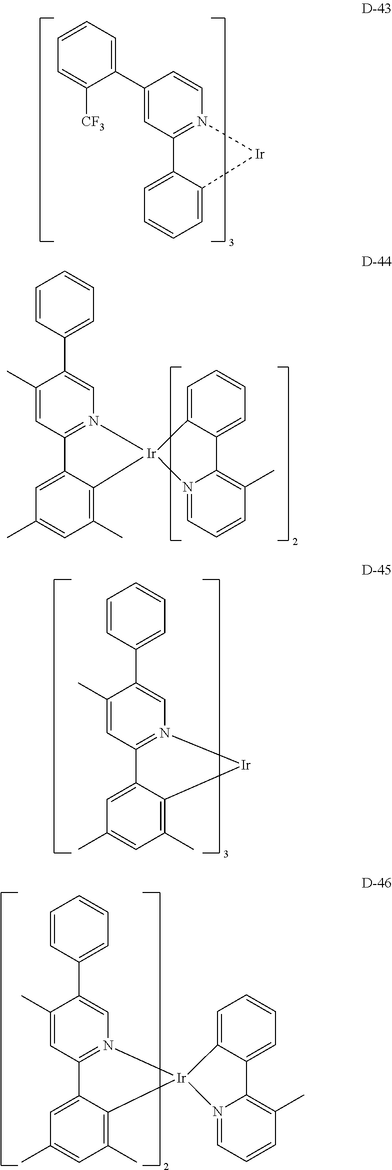

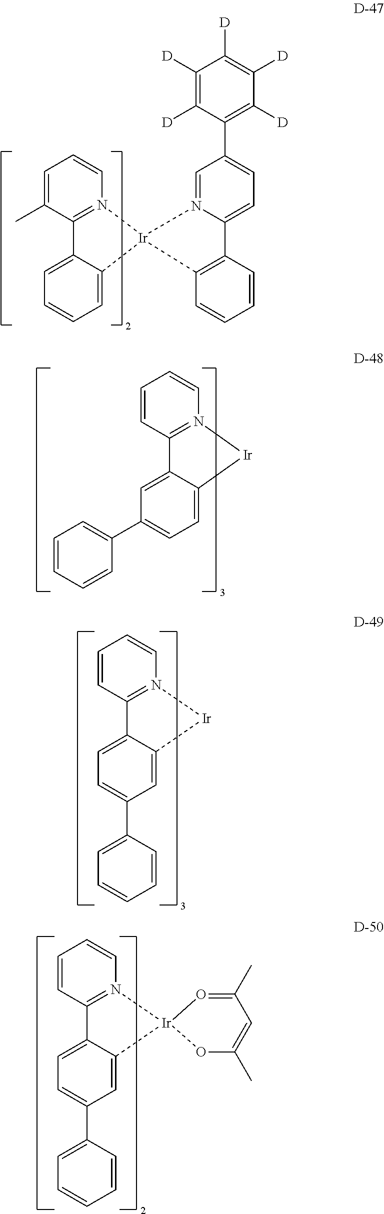

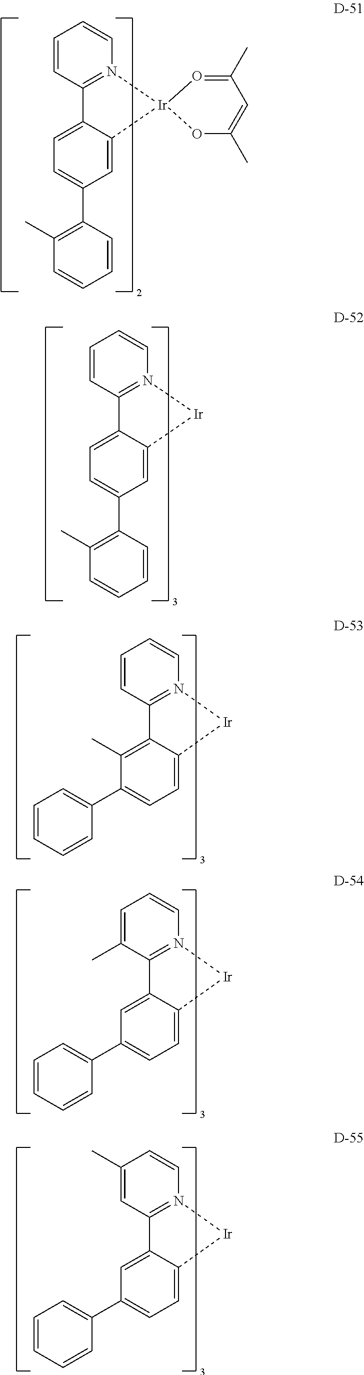

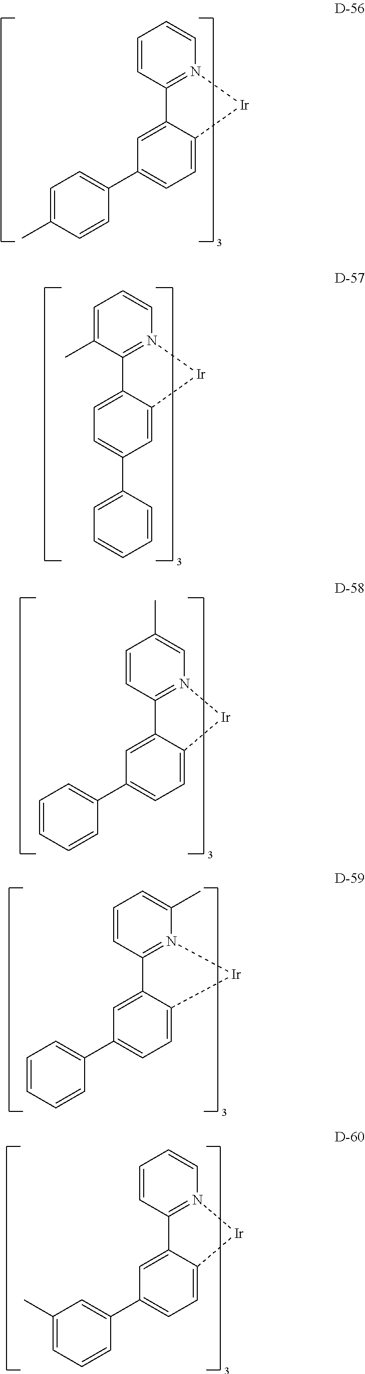

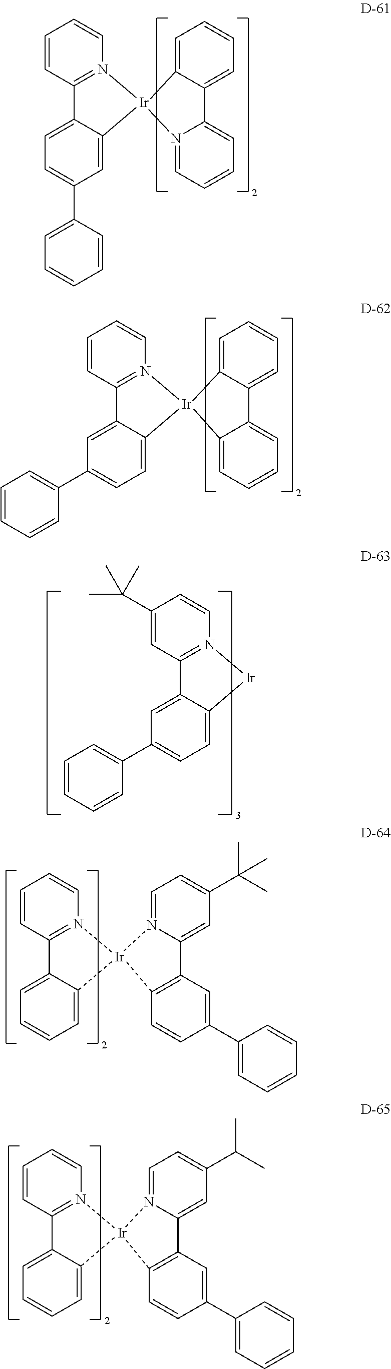

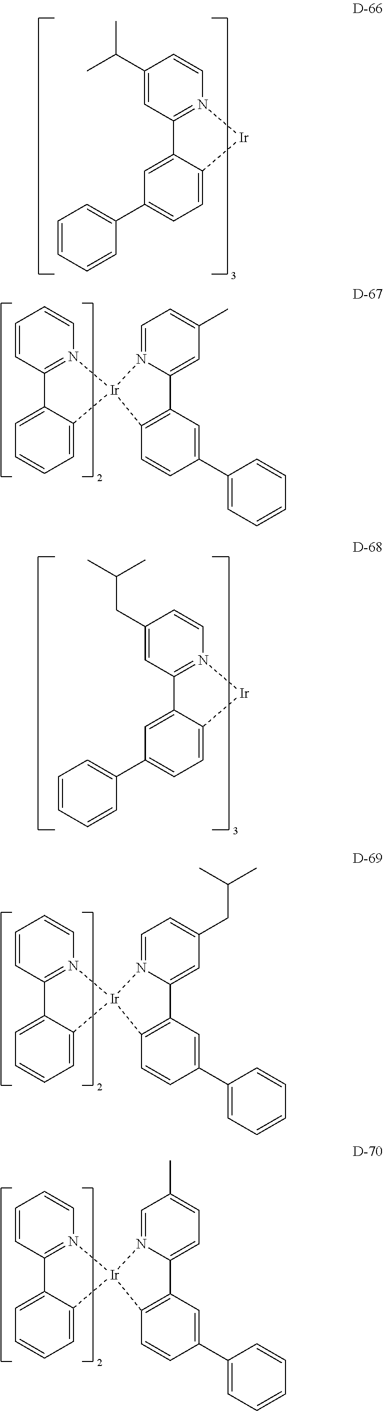

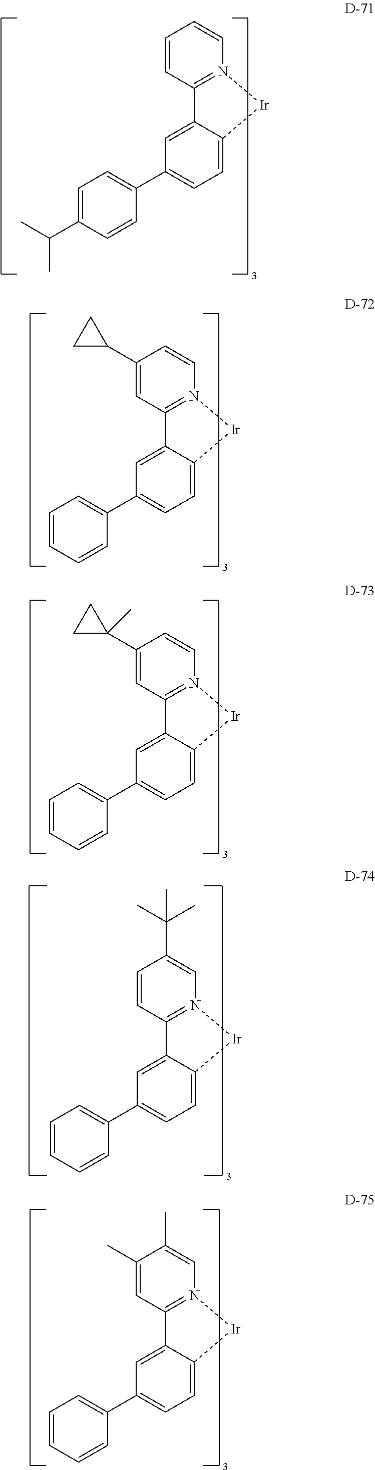

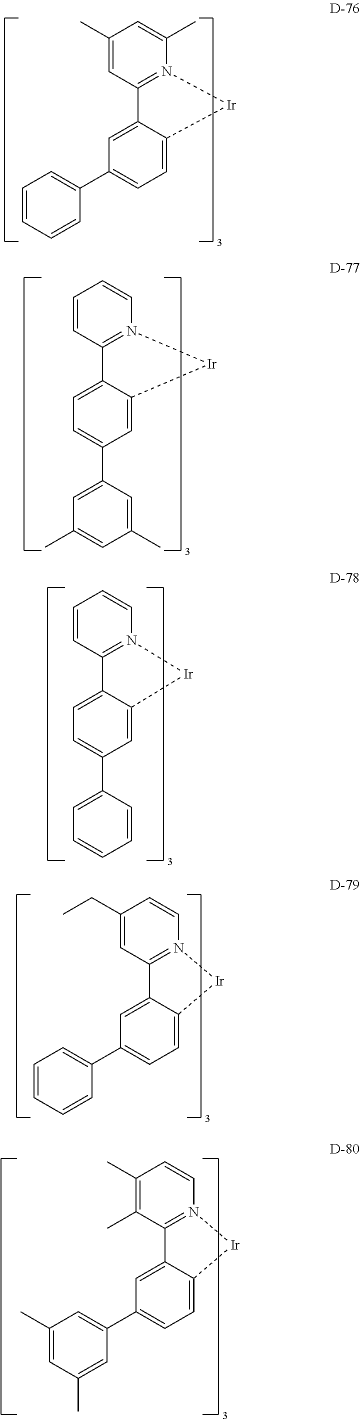

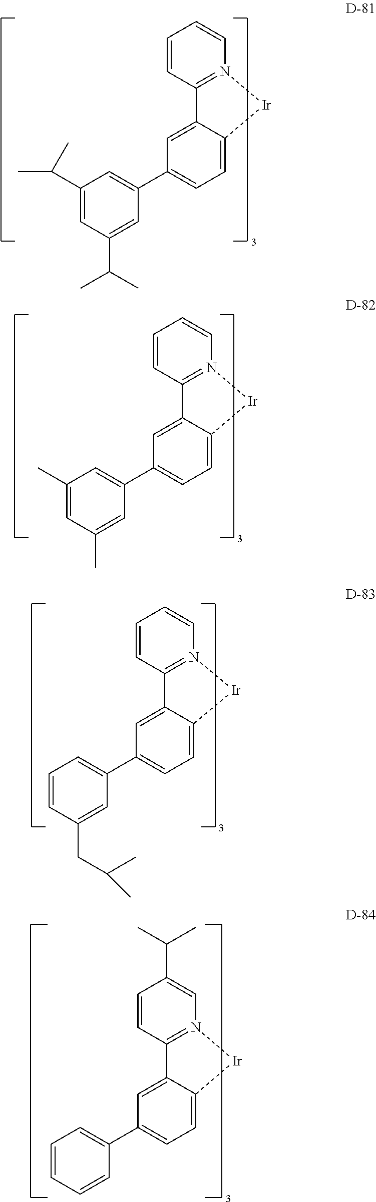

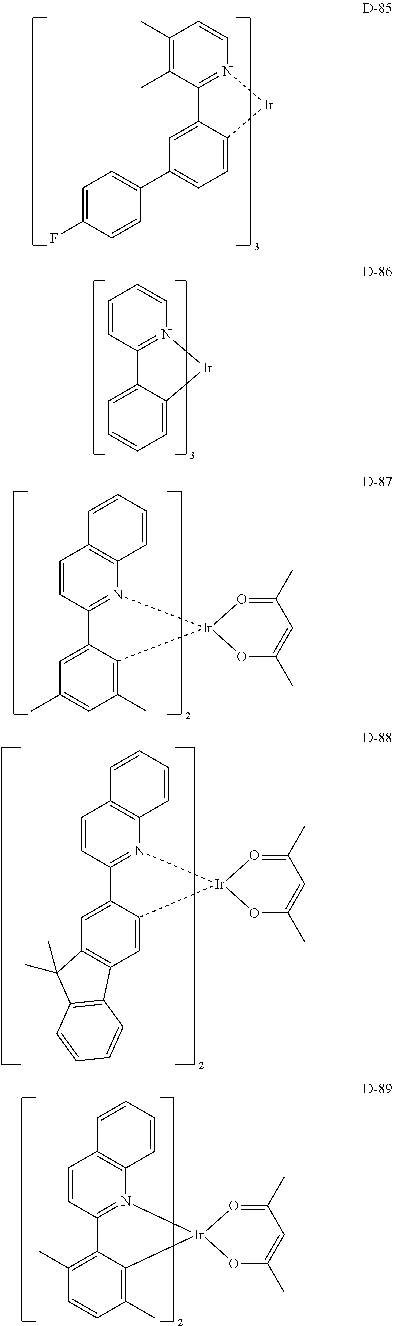

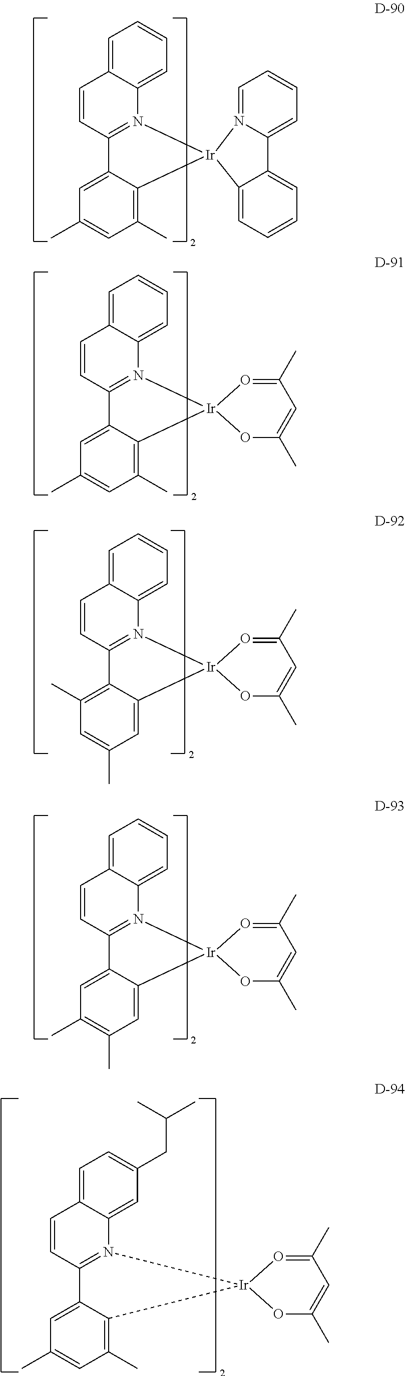

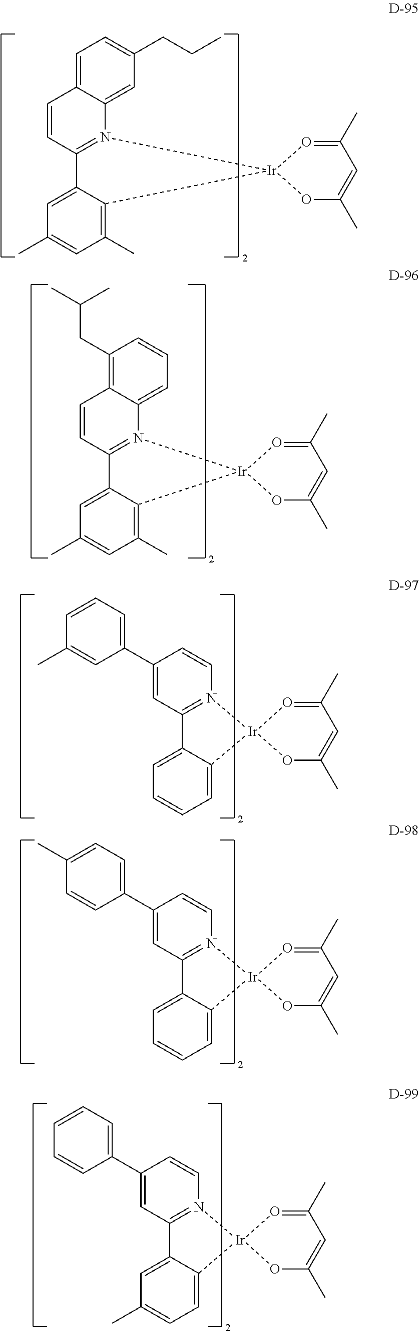

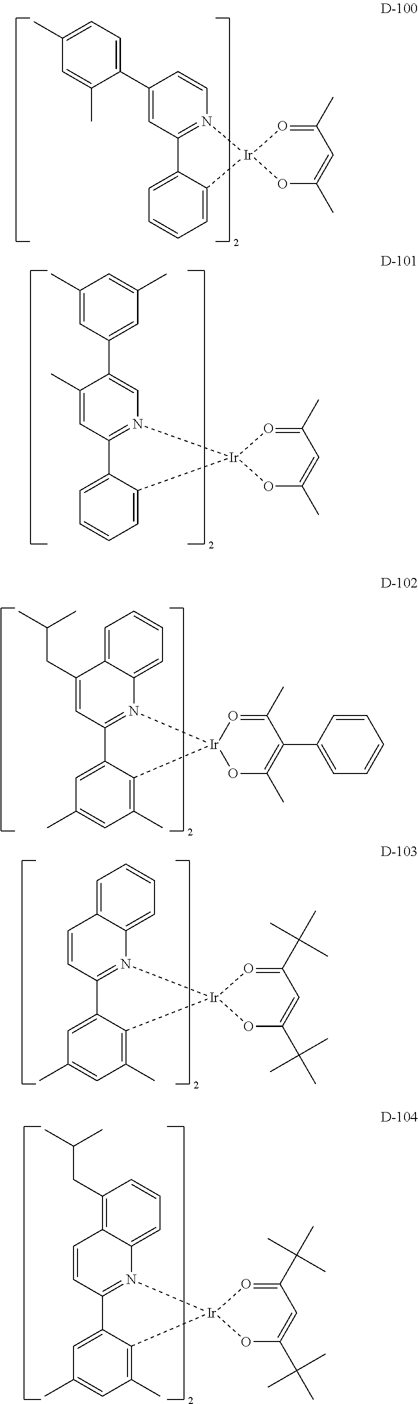

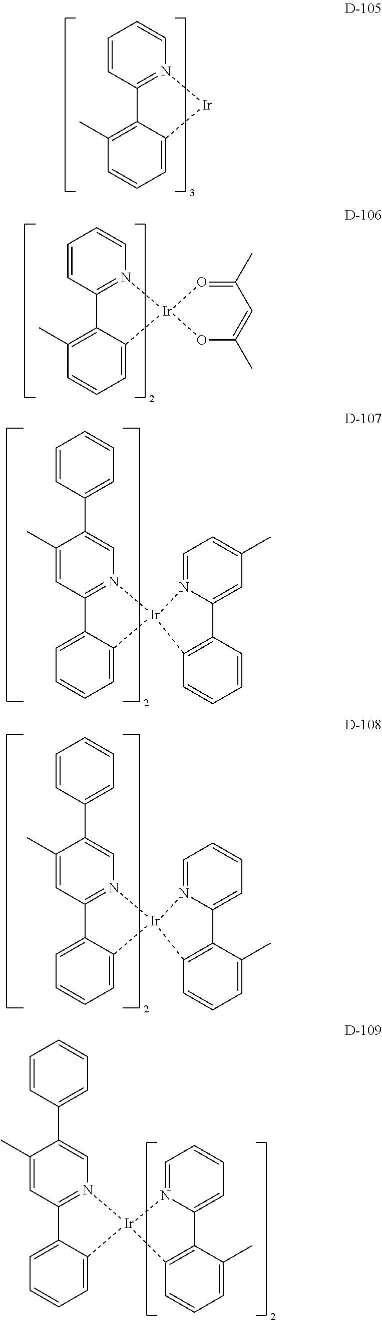

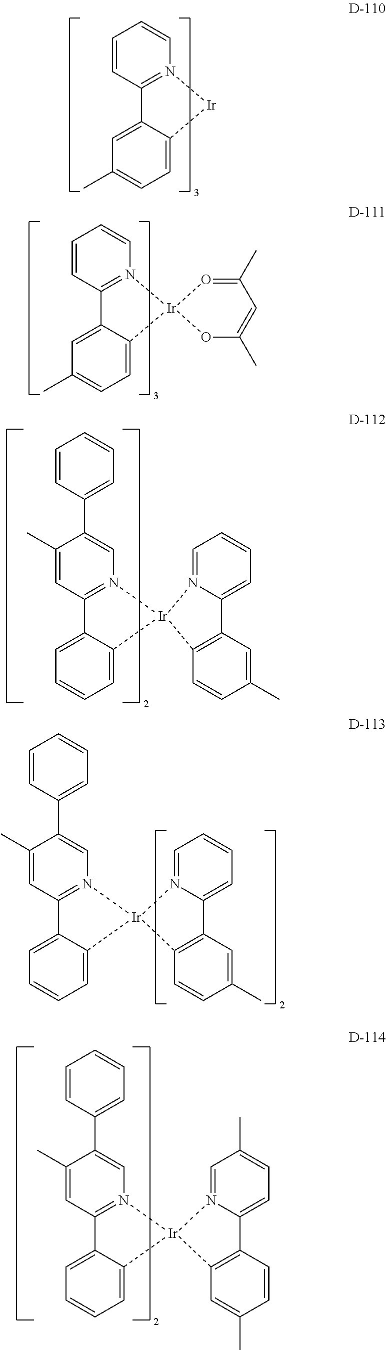

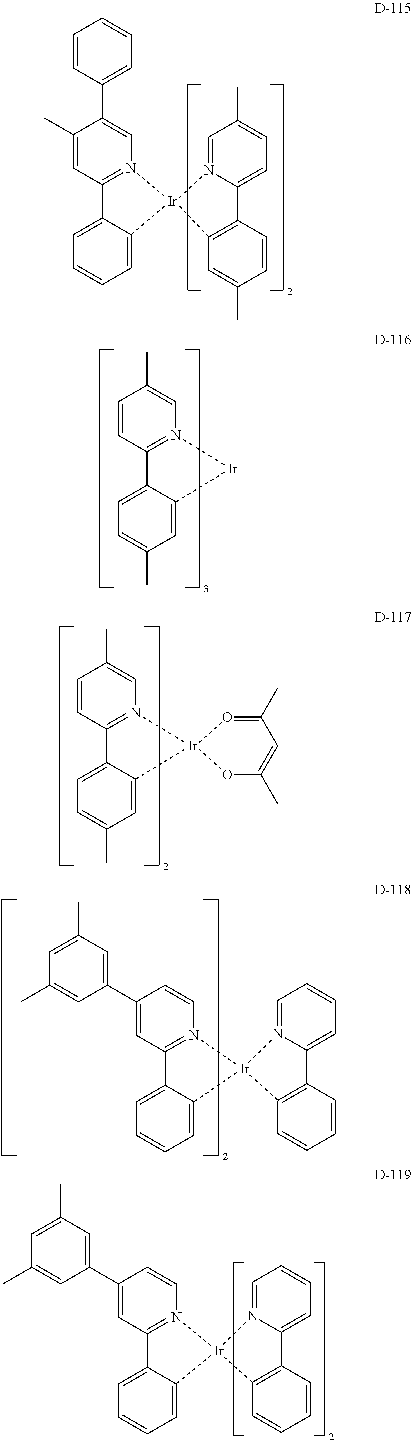

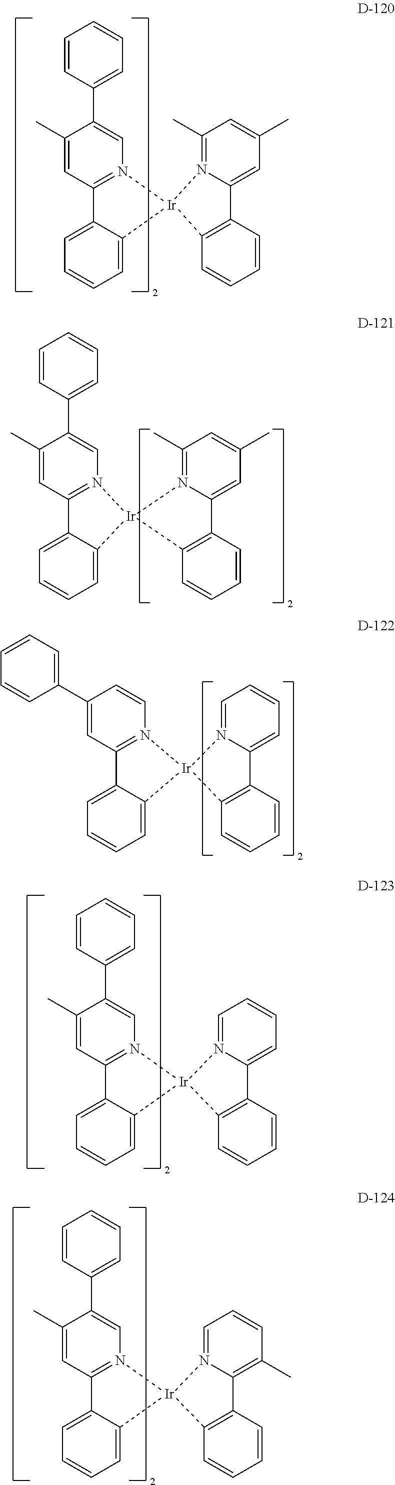

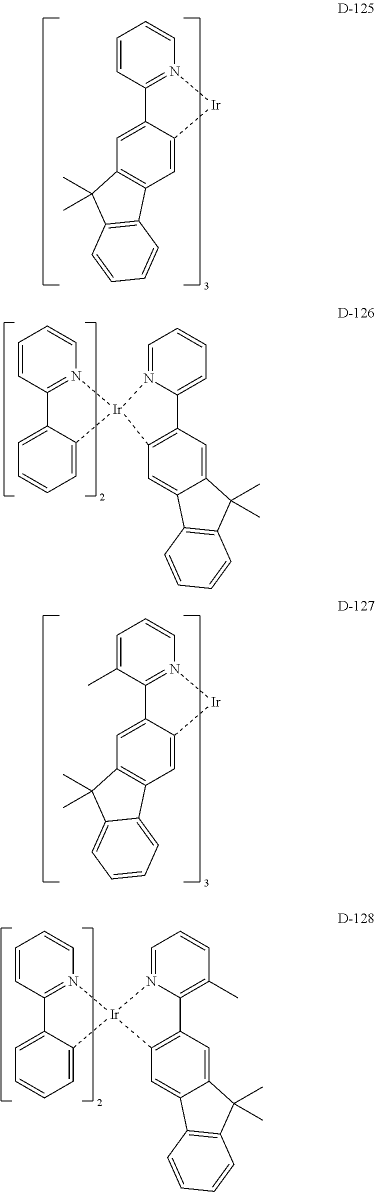

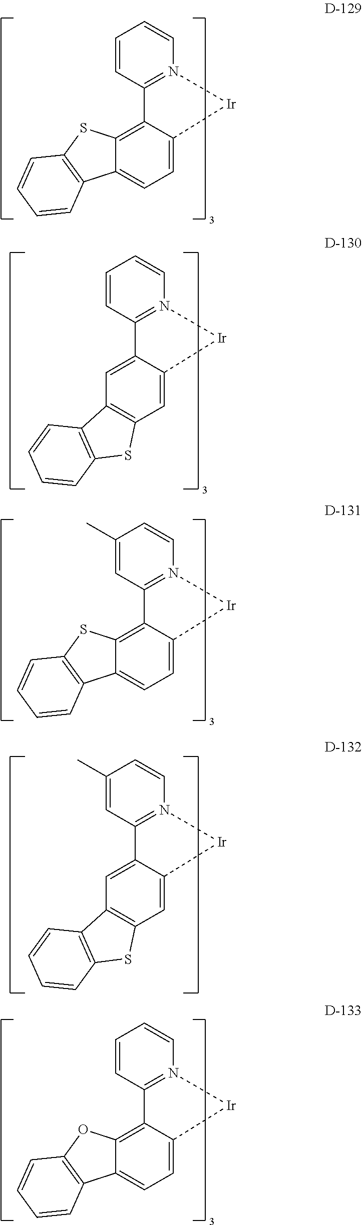

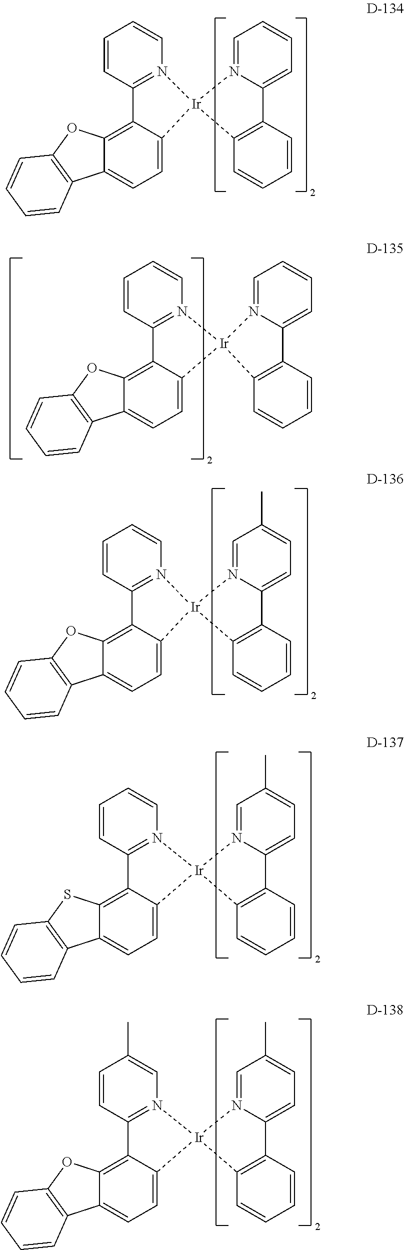

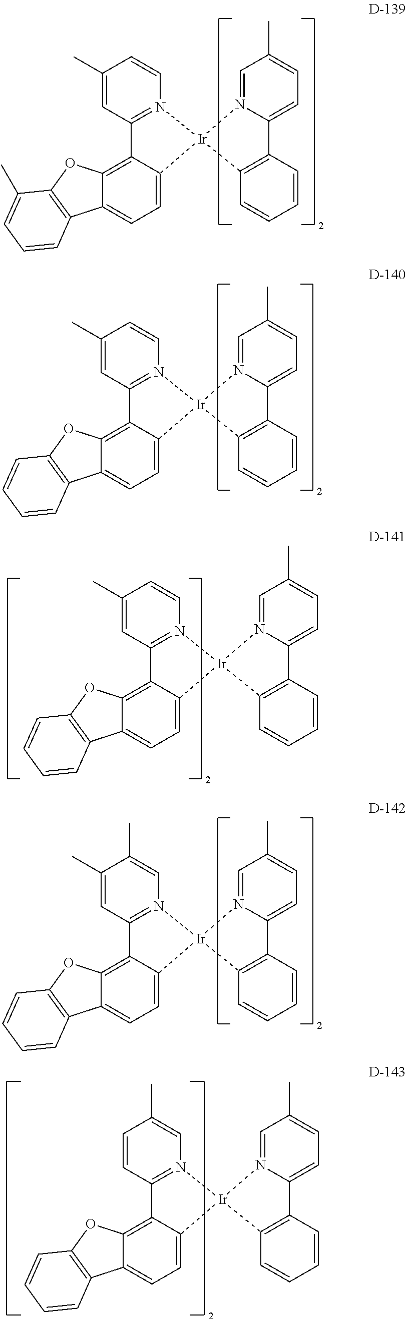

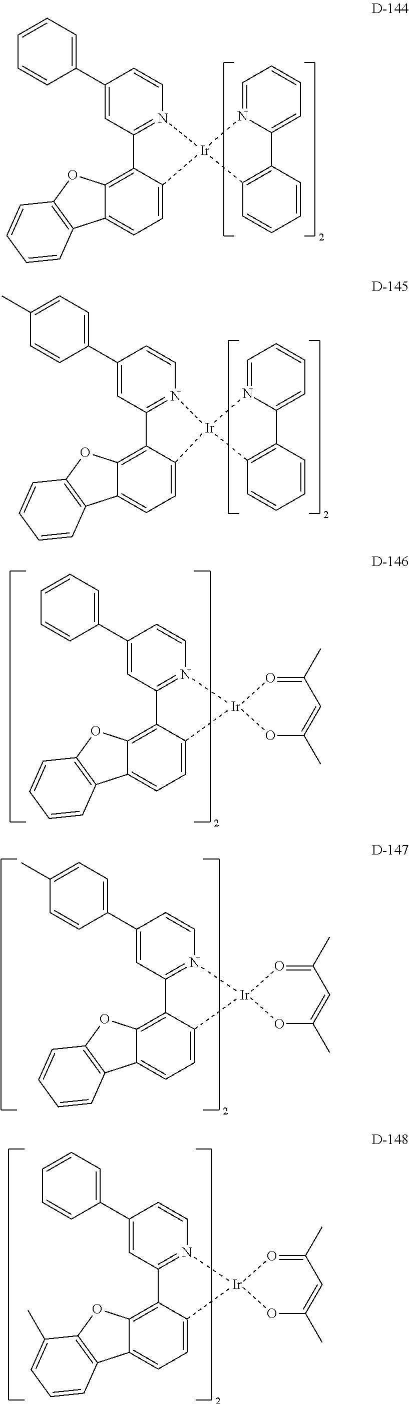

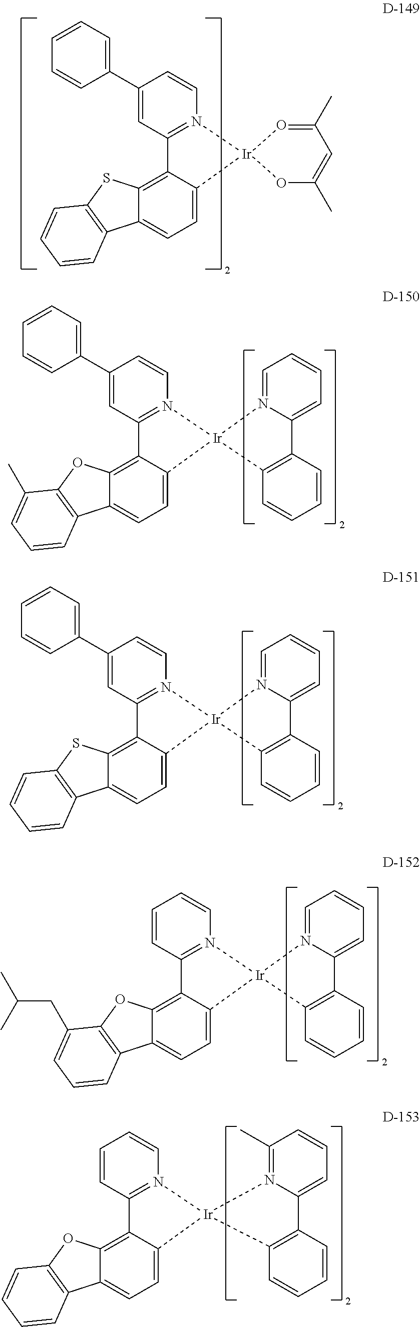

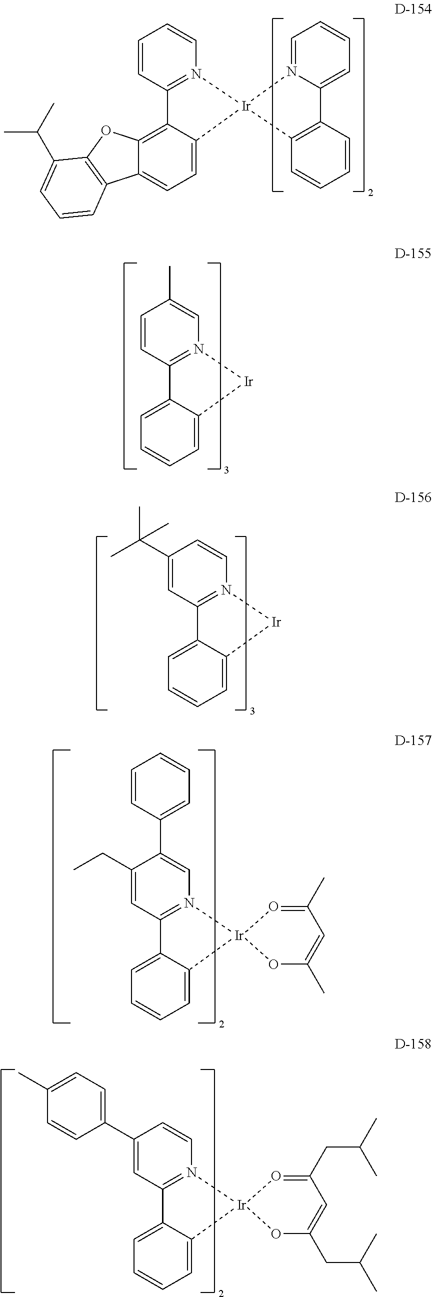

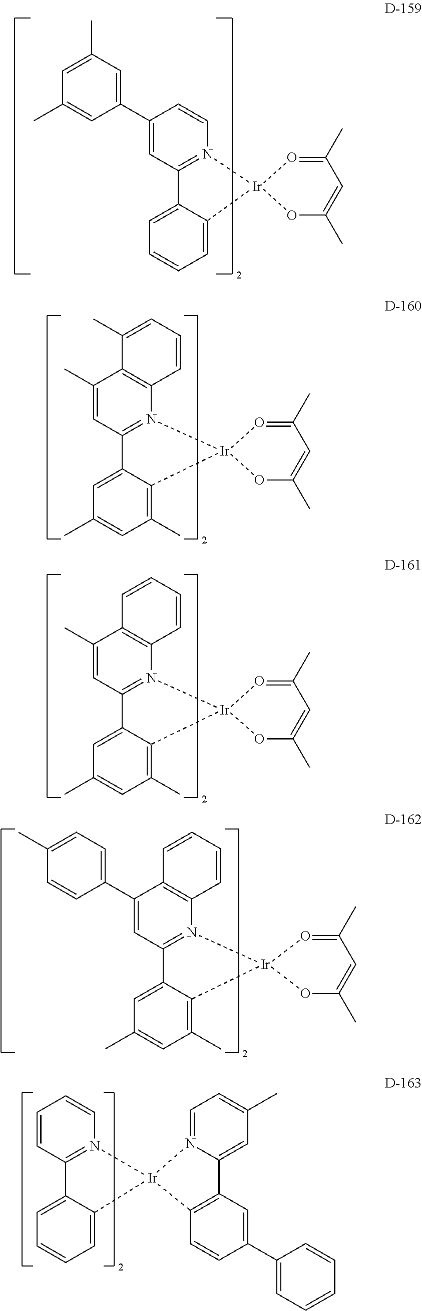

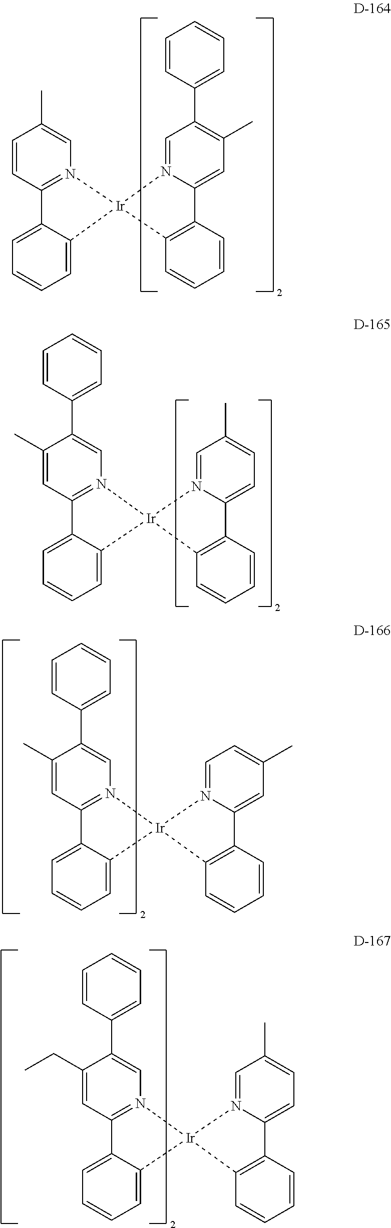

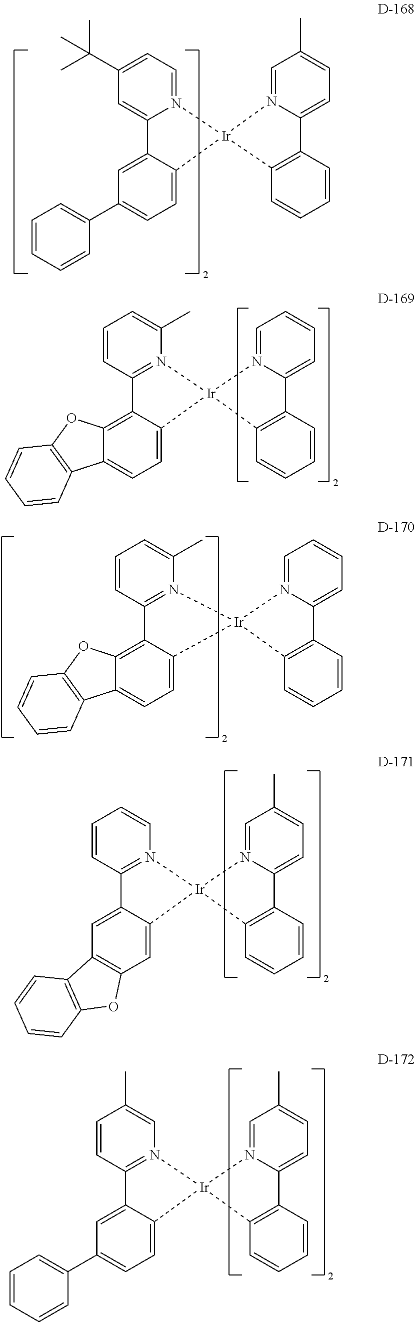

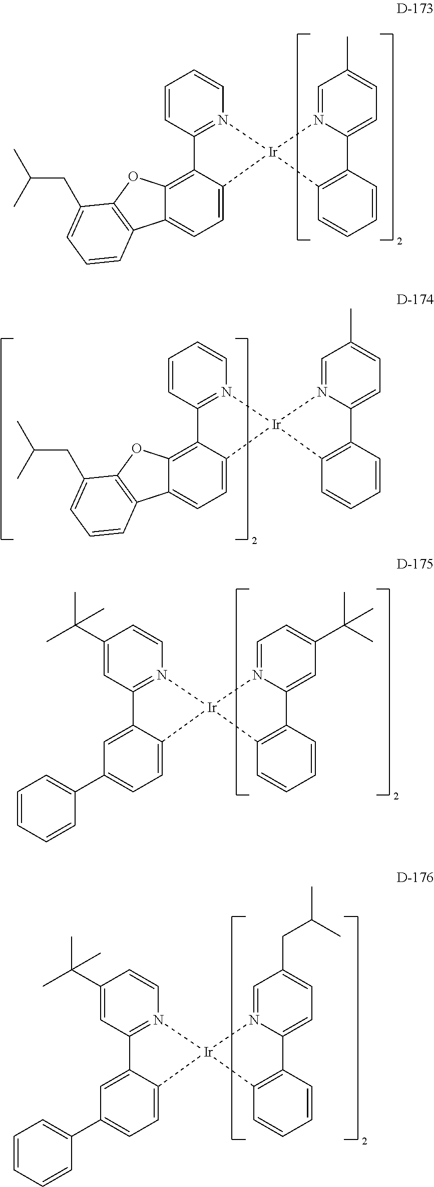

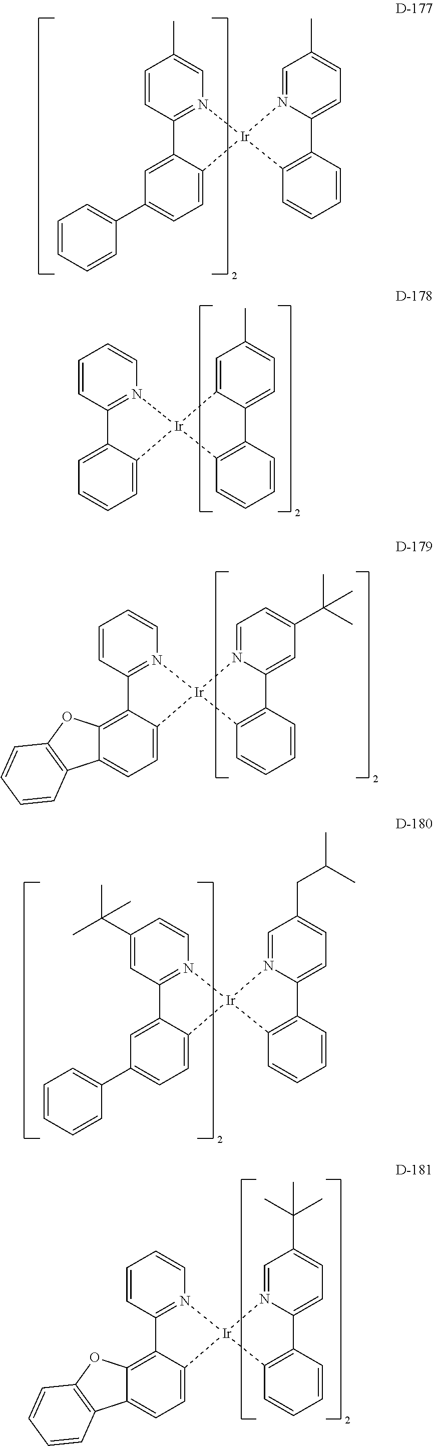

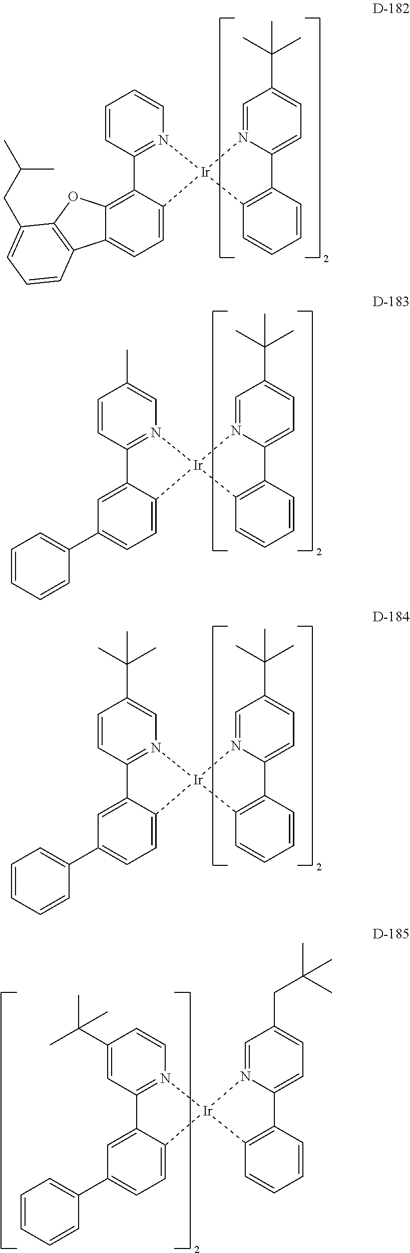

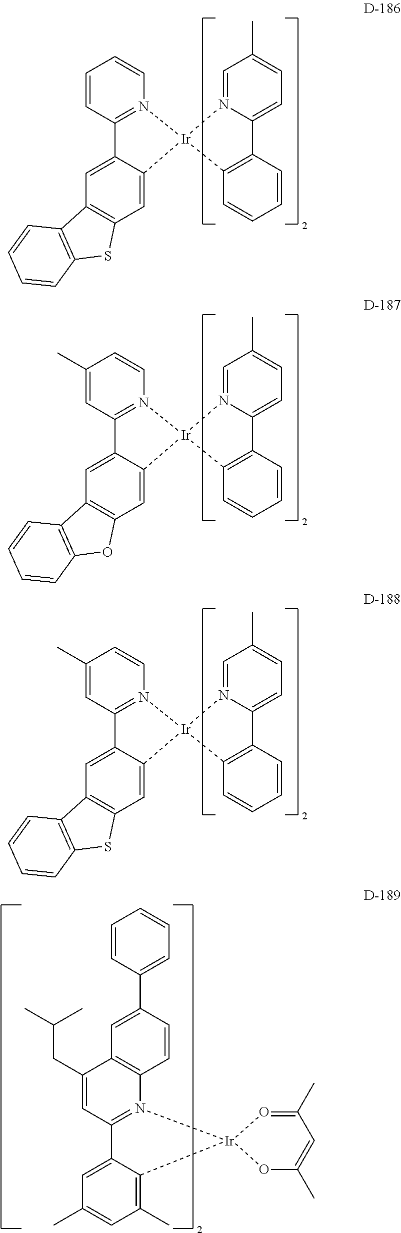

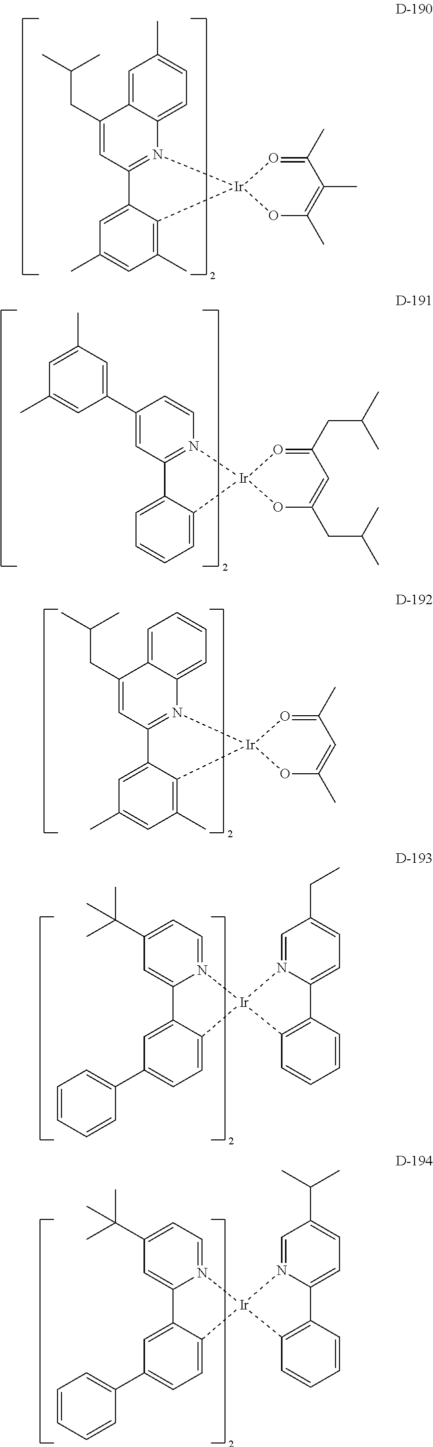

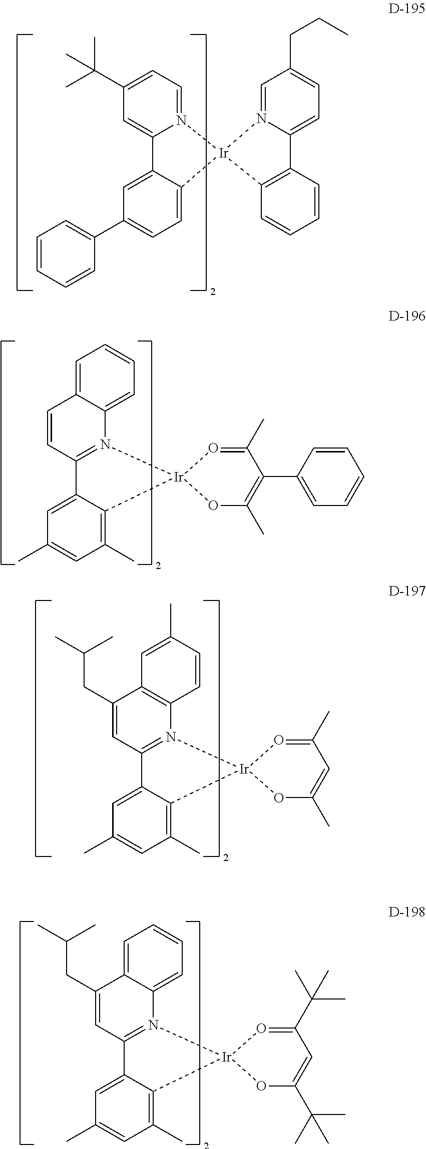

In another embodiment of the present invention, a composition for preparing an organic electroluminescent device is provided. The composition comprises the compound according to the present invention as a host material or a hole transport material.

In addition, the organic electroluminescent device according to the present invention comprises a first electrode; a second electrode; and at least one organic layer between the first and second electrodes. The organic layer comprises a light-emitting layer, and the light-emitting layer may comprise the composition for preparing the organic electroluminescent device according to the present invention.

The organic electroluminescent device according to the present invention may further comprise, in addition to the organic electroluminescent compound represented by formula 1, at least one compound selected from the group consisting of arylamine-based compounds and styrylarylamine-based compounds.

In the organic electroluminescent device according to the present invention, the organic layer may further comprise at least one metal selected from the group consisting of metals of Group 1, metals of Group 2, transition metals of the 4^{th} period, transition metals of the 5^{th} period, lanthanides and organic metals of d-transition elements of the Periodic Table, or at least one complex compound comprising said metal. The organic layer may further comprise a light-emitting layer and a charge generating layer.

In addition, the organic electroluminescent device according to the present invention may emit white light by further comprising at least one light-emitting layer which comprises a blue electroluminescent compound, a red electroluminescent compound or a green electroluminescent compound known in the field, besides the compound according to the present invention. Also, if necessary, a yellow or orange light-emitting layer can be comprised in the device.

According to the present invention, at least one layer (hereinafter, “a surface layer”) is preferably placed on an inner surface(s) of one or both electrode(s); selected from a chalcogenide layer, a metal halide layer, and a metal oxide layer. Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, said chalcogenide includes SiOₓ(1≦×≦2), AlOₓ(1≦×≦1.5), SiON, SiAION, etc.; said metal halide includes LiF, MgF₂, CaF₂, a rare earth metal fluoride, etc.; and said metal oxide includes Cs₂O, Li₂O, MgO, SrO, BaO, CaO, etc.

In the organic electroluminescent device according to the present invention, a mixed region of an electron transport compound and reductive dopant, or a mixed region of a hole transport compound and an oxidative dopant is preferably placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge generating layer to prepare an electroluminescent device having two or more electroluminescent layers and emitting white light.

In order to form each layer of the organic electroluminescent device according to the present invention, dry film-forming methods such as vacuum evaporation, sputtering, plasma and ion plating methods, or wet film-forming methods such as spin coating, dip coating, and flow coating methods can be used.

When using a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

Hereinafter, the organic electroluminescent compound, the preparation method of the compound, and the luminescent properties of the device will be explained in detail with reference to the following examples.

### EXAMPLE 1

### Preparation of Compound H-44

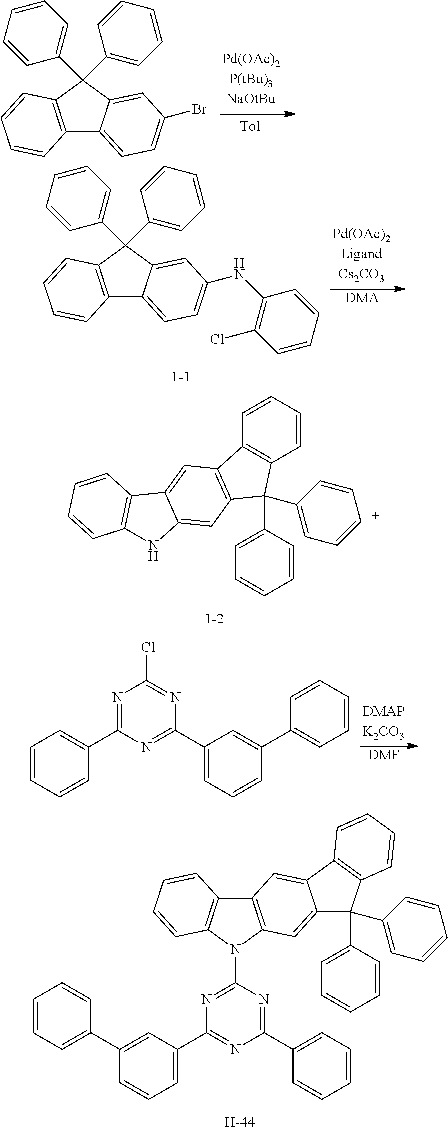

### Preparation of Compound 1-1

After introducing 2-bromo-9,9-diphenyl-9H-fluorene (8 g, 0.020 mol), 2-chloroaniline (3.1 mL, 0.030 mol), Pd(OAc)₂ (181 mg, 0.805 mmol), P(t-Bu)₃ (50% in toluene) (0.8 mL,3 1.61 mmol), NaOt-Bu (4.8 g, 0.056 mol), and toluene 58 mL in a flask, the resulting mixture was stirred at 140° C. for 4 hours. After completing the reaction, the mixture was washed with distilled water and extracted with ethyl acetate (EA). The organic layer was dried with MgSO₄, the solvent was removed with a rotary evaporator, and the residue was purified with column chromatography to obtain compound 1-1 (7.3 g, 82%).

### Preparation of Compound 1-2

After introducing compound 1-1 (7.3 g, 0.016 mol) in a flask, Pd(OAc)₂ (190 mg, 0.84 mmol), tricyclohexylphosphonium tetrafluoroborate (620 mg, 0.0016 mol), Cs₂CO₃ (16 g, 0.050 mol), and dimethylacetamide (DMA) 85 mL were added. The reactant mixture was heated to 190° C. and stirred for 5 hours. After completing the reaction, the mixture was washed with distilled water and extracted with EA. The organic layer was dried with MgSO₄, the solvent was removed with a rotary evaporator, and the residue was purified with column chromatography to obtain compound 1-2 (4.8 g, 59%).

### Preparation of Compound H-44

After introducing compound 1-2 (4.8 g, 0.011 mol) in a flask, 2-([1,1′-biphenyl]-3-yl)-4-chloro-6-phenyl-1,3,5-triazine (4.8 g, 0.014 mol), dimethylaminopyridine (DMAP) (720 mg, 0.005 mmol), K₂CO₃ (4.0 g, 0.029 mol), and dimethylformamide (DMF) 120 mL were added. The reactant mixture was heated to 120° C. and stirred for 3 hours. After completing the reaction, the mixture was washed with distilled water and extracted with EA. The organic layer was dried with MgSO₄, the solvent was removed with a rotary evaporator, and the residue was purified with column chromatography to obtain compound H-44 (6.9 g, 82%).

Compounds H-32, H-44, H-55, H-56, H-57, and H-58 were prepared using the method of Example 1. Yield (%), melting point (° C.), UV spectrum (nm), PL spectrum (nm), and molecular weight of the produced compounds are shown in the following Table.

| | | Melting | UV spectrum | | |
|---|---|---|---|---|---|
| | Yield | Point | (nm, | PL spectrum | Molecular |
| Compound | (%) | (° C.) | toluene) | (nm, toluene) | Weight |
| H-32 | 23 | 198 | 258 (MC) | 535 (MC) | 590.00 |
| H-44 | 47 | 326 | 334 | 486 | 714 |
| H-55 | 47 | 382 | 361 (MC) | 514 (MC) | 790 |
| H-56 | 29 | 371 | 257 (MC) | 543 (MC) | 744.80 |
| H-57 | 57 | 363 | 238 (MC) | 532 (MC) | 728.00 |
| H-58 | 30 | 267 | 254 (MC) | 493 (MC) | 713.00 |

[wherein MC represents methylene chloride]

### DEVICE EXAMPLE 1

### Production of an OLED Device Using the Organic Electroluminescent Compound According to the Present Invention

An OLED device was produced using the organic electroluminescent compound according to the present invention. A transparent electrode indium tin oxide (ITO) thin film (15 Ω/sq) on a glass substrate for an organic light-emitting diode (OLED) device (Geomatec, Japan) was subjected to an ultrasonic washing with trichloroethylene, acetone, ethanol, and distilled water, sequentially, and was then stored in isopropanol. Next, the ITO substrate was mounted on a substrate holder of a vacuum vapor depositing apparatus. N⁴,N⁴′-diphenyl-N⁴,N⁴′-bis(9-phenyl-9H-carbazol-3-yl)-[1,1′-biphenyl]-4,4′-diamine (compound Hl-1) was introduced into a cell of said vacuum vapor depositing apparatus, and then the pressure in the chamber of said apparatus was controlled to 10⁻⁶ torr. Thereafter, an electric current was applied to the cell to evaporate the above introduced material, thereby forming a first hole injection layer having a thickness of 80 nm on the ITO substrate. 1,4,5,8,9,12-hexaazatriphenylene-hexacarbonitrile (compound HI-2) was then introduced into another cell of said vacuum vapor depositing apparatus, and was evaporated by applying an electric current to the cell, thereby forming a second hole injection layer having a thickness of 3 nm on the first hole injection layer. N-([1,1′-biphenyl]-4-yl)-9,9-dimethyl-N-(4-(9-phenyl-9H-carbazol-3-yl)phenyl)-9H-fluorene-2-amine (compound HT-1) was introduced into another cell of said vacuum vapor depositing apparatus, and was evaporated by applying an electric current to the cell, thereby forming a hole transport layer having a thickness of 40 nm on the second hole injection layer. Thereafter, compound H-44 was introduced into one cell of the vacuum vapor depositing apparatus as a host material, and compound D-1 was introduced into another cell as a dopant. The two materials were evaporated at different rates and were deposited in a doping amount of 15 wt % (the amount of dopant) based on the total amount of the host and dopant to form a light-emitting layer having a thickness of 40 nm on the hole transport layer. 2,4-bis(9,9-dimethyl-9H-fluoren-2-yl)-6-(naphthalen-2-yl)-1,3,5-triazine (compound ET-1) and lithium quinolate (compound E1-1) were then introduced into another two cells, evaporated at the rate of 4:6, and deposited to form an electron transport layer having a thickness of 35 nm on the light-emitting layer. Next, after depositing lithium quinolate as an electron injection layer having a thickness of 2 nm on the electron transport layer, an Al cathode having a thickness of 80 nm was deposited by another vacuum vapor deposition apparatus on the electron injection layer. Thus, an OLED device was produced. All the materials used for producing the OLED device were purified by vacuum sublimation at 10⁻⁶ torr prior to use.

The produced OLED device showed a green emission of which the time taken for the luminance at 15,000 nit to be reduced from 100% to 95% at a constant current was 13.7 hours.

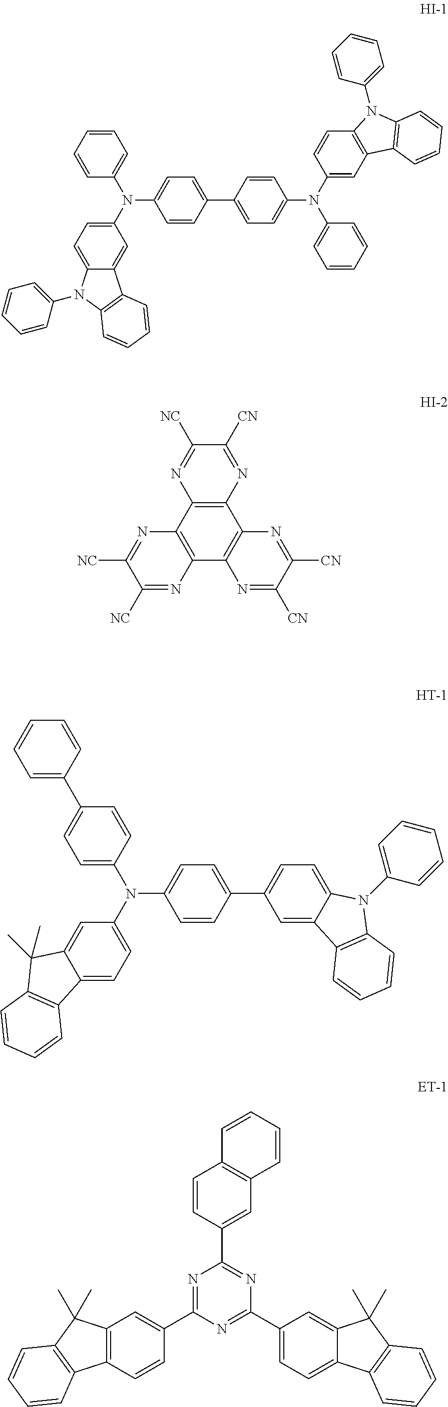 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(-c3nc(-c4ccc5c(c4)C(C)(C)c4ccccc4-5)nc(-c4ccc5ccccc5c4)n3)cc21]

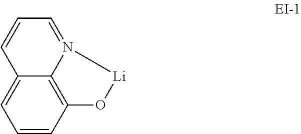

### DEVICE EXAMPLE 2

### Production of an OLED Device Using the Organic Electroluminescent Compound According to the Present Invention

An OLED device was produced in the same manner as in Device Example 1, except for using compound H-58 for the host as the light-emitting material. The produced OLED device showed a green emission of which the time taken for the luminance at 15,000 nit to be reduced from 100% to 95% at a constant current was 8.9 hours.

### COMPARATIVE EXAMPLE 1

### Production of an OLED Device Using a Conventional Organic Electroluminescent Compound

An OLED device was produced in the same manner as in Device Example 1, except for using the compound below for the host as the light-emitting material.

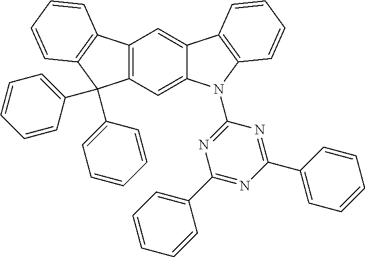 [SMILES: c1ccc(-c2nc(-c3ccccc3)nc(-n3c4ccccc4c4cc5c(cc43)C(c3ccccc3)(c3ccccc3)c3ccccc3-5)n2)cc1]

The produced OLED device showed a green emission of which the time taken for the luminance at 15,000 nit to be reduced from 100% to 95% at a constant current was 6.6 hours.

It is verified that the lifespan characteristics of the organic electroluminescent compound according to the present invention are superior to conventional materials.

1. An organic electroluminescent compound represented by the following formula 1: 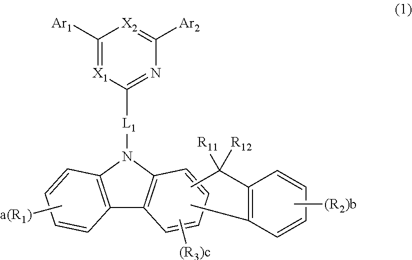
wherein
X₁ and X₂ each independently represent CH or N;
L₁ represents a single bond, a substituted or unsubstituted (C6-C30)arylene, or a substituted or unsubstituted 5- to 30-membered heteroarylene;
Ar₁ represents a substituted or unsubstituted (C6-C18)aryl;
Ar₂ represents a substituted or unsubstituted (C6-C18)aryl, or a substituted or unsubstituted 5- to 15-membered heteroaryl;
Ar₁ and Ar₂ are different from each other;
R₁ and R₂ each independently represent hydrogen, deuterium, a halogen, a cyano, a substituted or unsubstituted (C1-C30)alkyl, a substituted or unsubstituted (C6-C30)aryl, a substituted or unsubstituted 5- to 30-membered heteroaryl, a substituted or unsubstituted (C6-C30)aryl(C1-C30)alkyl, a substituted or unsubstituted (C3-C30)cycloalkyl, a substituted or unsubstituted (C1-C30)alkoxy, a substituted or unsubstituted (C1-C30)alkylsilyl, a substituted or unsubstituted (C6-C30)arylsilyl, a substituted or unsubstituted (C6-C30)aryl(C1-C30)alkylsilyl, a substituted or unsubstituted (C1-C30)alkylamino, a substituted or unsubstituted (C6-C30)arylamino, or a substituted or unsubstituted (C1-C30)alkyl(C6-C30)arylamino; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur;
R₃ represents hydrogen, deuterium, a halogen, a cyano, a substituted or unsubstituted (C1-C30)alkyl, a substituted or unsubstituted (C6-C30)aryl, or a substituted or unsubstituted 5- to 30-membered heteroaryl; or are linked to an adjacent substituent(s) to form a mono- or polycyclic (C3-C30) alicyclic or aromatic ring, whose carbon atom(s) may be replaced with at least one hetero atom selected from nitrogen, oxygen, and sulfur;
R₁₁ and R₁₂ each independently represent a substituted or unsubstituted (C1-C30)alkyl, a substituted or unsubstituted (C6-C30)aryl, or a substituted or unsubstituted 5- to 30-membered heteroaryl; or are linked to each other to form a mono- or polycyclic (C3-C30) alicyclic or aromatic ring, whose carbon atom(s) may be replaced with at least one hetero atom selected from nitrogen, oxygen, and sulfur;
a and b each independently represent an integer of 1 to 4, where a or b is an integer of 2 or more, each of R₁ and each of R₂ may be the same or different;
c is an integer of 1 to 2, where c is 2, each of R₃ may be the same or different; and
the heteroaryl(ene) contains at least one hetero atom selected from B, N, O, S, Si, and P.

2. The organic electroluminescent compound according to claim 1, wherein formula 1 is represented by one of the following formulae 2 to 7: 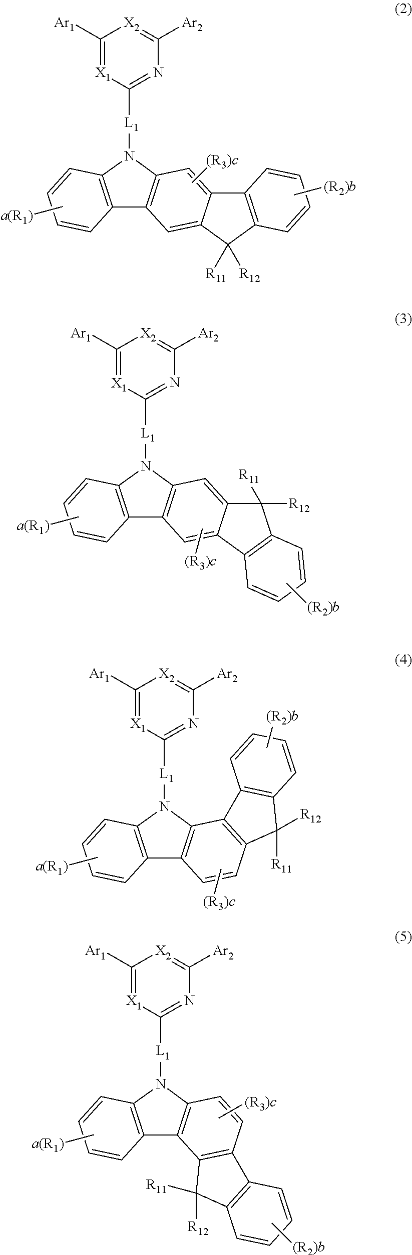 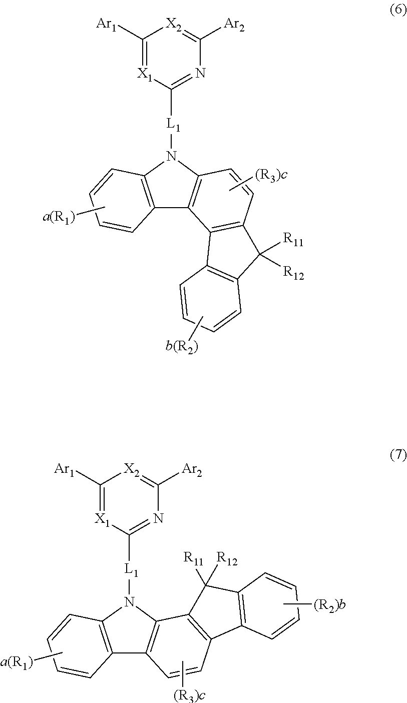
wherein
X₁, X₂, L₁, Ar₁, Ar₂, R₁ to R₃, R₁₁, R₁₂, a, b, and c are as defined in claim 1.

3. The organic electroluminescent compound according to claim 1, wherein the substituents of the substituted (C1-C30)alkyl, the substituted (C3-C30)cycloalkyl, the substituted (C6-C30)aryl(ene), the substituted 5- to 30-membered heteroaryl(ene), the substituted (C6-C30)aryl(C1-C30)alkyl, the substituted (C1-C30)alkoxy, the substituted (C1-C30)alkylsilyl, the substituted (C6-C30)arylsilyl, the substituted (C6-C30)aryl(C1-C30)alkylsilyl, the substituted (C1-C30)alkylamino, the substituted (C6-C30)arylamino, and the substituted (C1-C30)alkyl(C6-C30)arylamino in L₁, Ar₁, Ar₂, R₁ to R₃, R₁₁, and R₁₂ each independently are at least one selected from the group consisting of deuterium, a halogen, a cyano, a carboxyl, a nitro, a hydroxyl, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a 3- to 7-membered heterocycloalkyl, a (C6-C30)aryloxy, a (C6-C30)arylthio, a 5- to 30-membered heteroaryl unsubstituted or substituted with a (C6-C30)aryl, a (C6-C30)aryl unsubstituted or substituted with a 5- to 30-membered heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

4. The organic electroluminescent compound according to claim 1, wherein
X₁ and X₂ each independently represent CH or N;
L₁ represents a single bond, or a substituted or unsubstituted (C6-C12)arylene;
Ar₁ represents a substituted or unsubstituted (C6-C18)aryl;
Ar₂ represents a substituted or unsubstituted (C6-C18)aryl, or a substituted or unsubstituted 5- to 15-membered heteroaryl;
Ar₁ and Ar₂ are different from each other;
R₁ and R₂ each independently represent hydrogen, a substituted or unsubstituted (C6-C12)aryl, or a substituted or unsubstituted 5- to 15-membered heteroaryl;
R₃ represents hydrogen; and
R₁₁ and R₁₂ each independently represent a substituted or unsubstituted (C1-C6)alkyl, or a substituted or unsubstituted (C6-C12)aryl; or are linked to each other to form a mono- or polycyclic (C3-C15) alicyclic or aromatic ring.

5. The organic electroluminescent compound according to claim 1, wherein
X₁ and X₂ each independently represent CH or N;
L₁ represents a single bond, or an unsubstituted (C6-C12)arylene;
Ar₁ represents a (C6-C18)aryl unsubstituted or substituted with a cyano, a (C1-C6)alkyl, a (C6-C12)aryl, or a 5- to 15-membered heteroaryl;
Ar₂ represents a (C6-C18)aryl unsubstituted or substituted with a cyano, a (C1-C6)alkyl, a (C6-C12)aryl, or a 5- to 15-membered heteroaryl; or a 5- to 15-membered heteroaryl unsubstituted or substituted with a (C6-C12)aryl;
Ar₁ and Ar₂ are different from each other;
R₁ and R₂ each independently represent hydrogen, an unsubstituted (C6-C12)aryl, or a 5- to 15-membered heteroaryl unsubstituted or substituted with a (C6-C12)aryl;
R₃ represents hydrogen; and
R₁₁ and R₁₂ each independently represent an unsubstituted (C1-C6)alkyl, or an unsubstituted (C6-C12)aryl; or are linked to each other to form a mono- or polycyclic (C3-C15) aromatic ring.

6. The organic electroluminescent compound according to claim 1, wherein Ar₁ and Ar₂ each independently represent a substituted or unsubstituted phenyl, a substituted or unsubstituted biphenyl, a substituted or unsubstituted terphenyl, a substituted or unsubstituted dibenzothiophene, a substituted or unsubstituted dibenzofuran, a substituted or unsubstituted carbazole, or a substituted or unsubstituted fluorene.

7. The organic electroluminescent compound according to claim 1, wherein the compound represented by formula 1 is selected from the group consisting of:

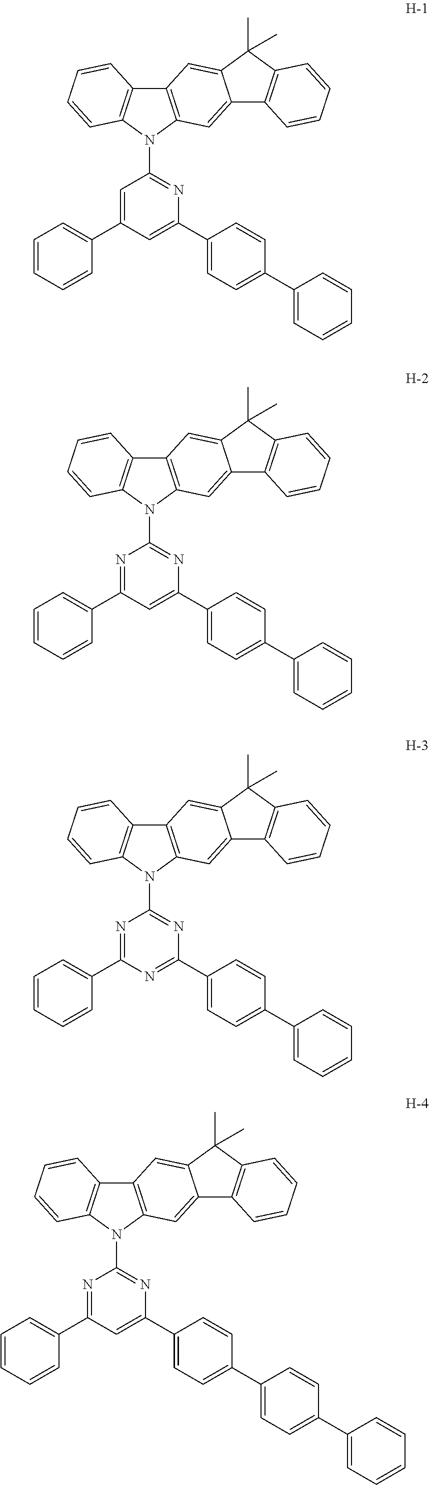 [SMILES: CC1(C)c2ccccc2-c2cc3c(cc21)c1ccccc1n3-c1cc(-c2ccccc2)cc(-c2ccc(-c3ccccc3)cc2)n1 ; CC1(C)c2ccccc2-c2cc3c(cc21)c1ccccc1n3-c1nc(-c2ccccc2)cc(-c2ccc(-c3ccccc3)cc2)n1 ; CC1(C)c2ccccc2-c2cc3c(cc21)c1ccccc1n3-c1nc(-c2ccccc2)nc(-c2ccc(-c3ccccc3)cc2)n1 ; CC1(C)c2ccccc2-c2cc3c(cc21)c1ccccc1n3-c1nc(-c2ccccc2)cc(-c2ccc(-c3ccc(-c4ccccc4)cc3)cc2)n1]

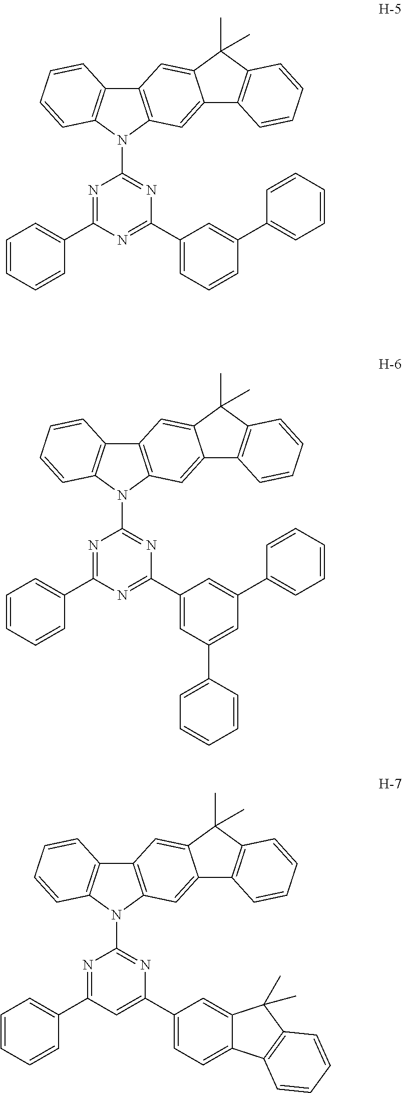 [SMILES: CC1(C)c2ccccc2-c2cc3c(cc21)c1ccccc1n3-c1nc(-c2ccccc2)nc(-c2cccc(-c3ccccc3)c2)n1 ; CC1(C)c2ccccc2-c2cc3c(cc21)c1ccccc1n3-c1nc(-c2ccccc2)nc(-c2cc(-c3ccccc3)cc(-c3ccccc3)c2)n1 ; CC1(C)c2ccccc2-c2ccc(-c3cc(-c4ccccc4)nc(-n4c5ccccc5c5cc6c(cc54)-c4ccccc4C6(C)C)n3)cc21]

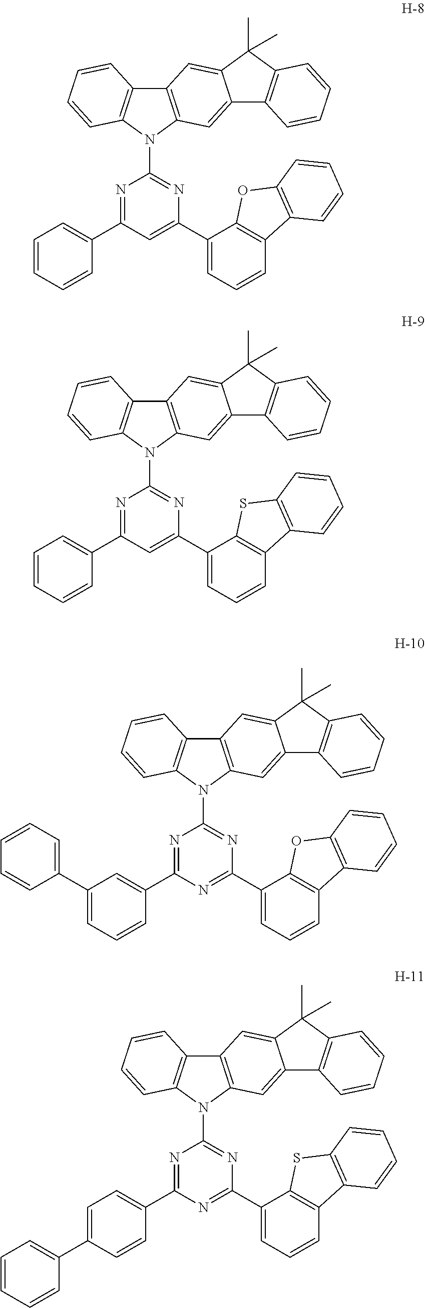

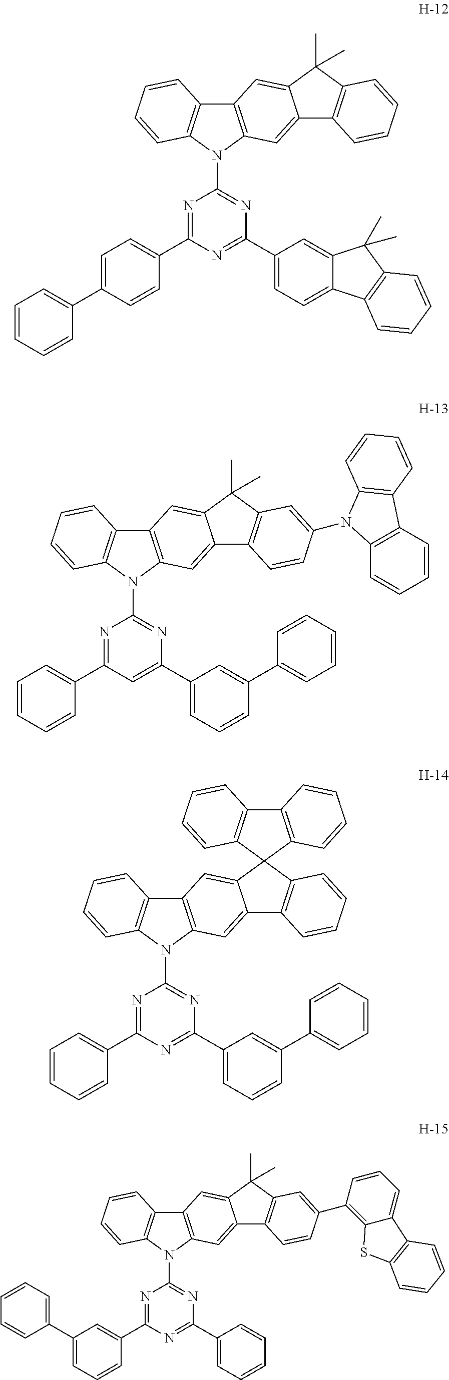 [SMILES: CC1(C)c2ccccc2-c2ccc(-c3nc(-c4ccc(-c5ccccc5)cc4)nc(-n4c5ccccc5c5cc6c(cc54)-c4ccccc4C6(C)C)n3)cc21 ; CC1(C)c2cc(-n3c4ccccc4c4ccccc43)ccc2-c2cc3c(cc21)c1ccccc1n3-c1nc(-c2ccccc2)cc(-c2cccc(-c3ccccc3)c2)n1 ; c1ccc(-c2cccc(-c3nc(-c4ccccc4)nc(-n4c5ccccc5c5cc6c(cc54)-c4ccccc4C64c5ccccc5-c5ccccc54)n3)c2)cc1 ; CC1(C)c2cc(-c3cccc4c3sc3ccccc34)ccc2-c2cc3c(cc21)c1ccccc1n3-c1nc(-c2ccccc2)nc(-c2cccc(-c3ccccc3)c2)n1]

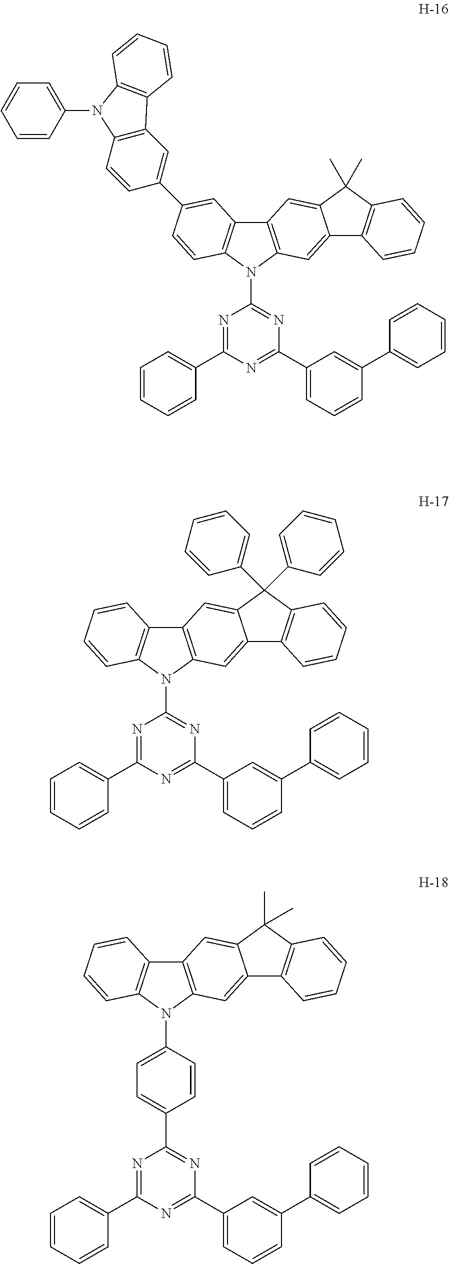 [SMILES: CC1(C)c2ccccc2-c2cc3c(cc21)c1cc(-c2ccc4c(c2)c2ccccc2n4-c2ccccc2)ccc1n3-c1nc(-c2ccccc2)nc(-c2cccc(-c3ccccc3)c2)n1 ; c1ccc(-c2cccc(-c3nc(-c4ccccc4)nc(-n4c5ccccc5c5cc6c(cc54)-c4ccccc4C6(c4ccccc4)c4ccccc4)n3)c2)cc1 ; CC1(C)c2ccccc2-c2cc3c(cc21)c1ccccc1n3-c1ccc(-c2nc(-c3ccccc3)nc(-c3cccc(-c4ccccc4)c3)n2)cc1]

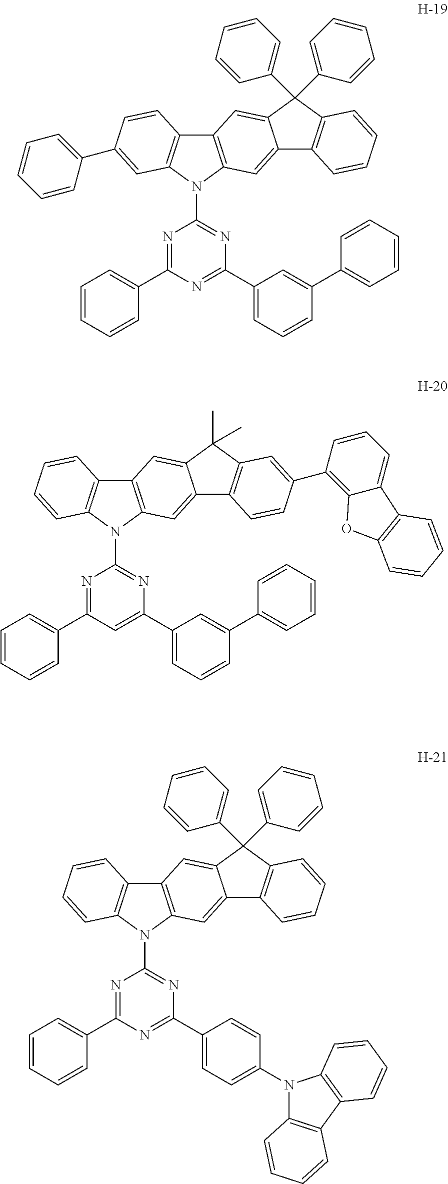 [SMILES: c1ccc(-c2cccc(-c3nc(-c4ccccc4)nc(-n4c5cc(-c6ccccc6)ccc5c5cc6c(cc54)-c4ccccc4C6(c4ccccc4)c4ccccc4)n3)c2)cc1 ; CC1(C)c2cc(-c3cccc4c3oc3ccccc34)ccc2-c2cc3c(cc21)c1ccccc1n3-c1nc(-c2ccccc2)cc(-c2cccc(-c3ccccc3)c2)n1 ; c1ccc(-c2nc(-c3ccc(-n4c5ccccc5c5ccccc54)cc3)nc(-n3c4ccccc4c4cc5c(cc43)-c3ccccc3C5(c3ccccc3)c3ccccc3)n2)cc1]

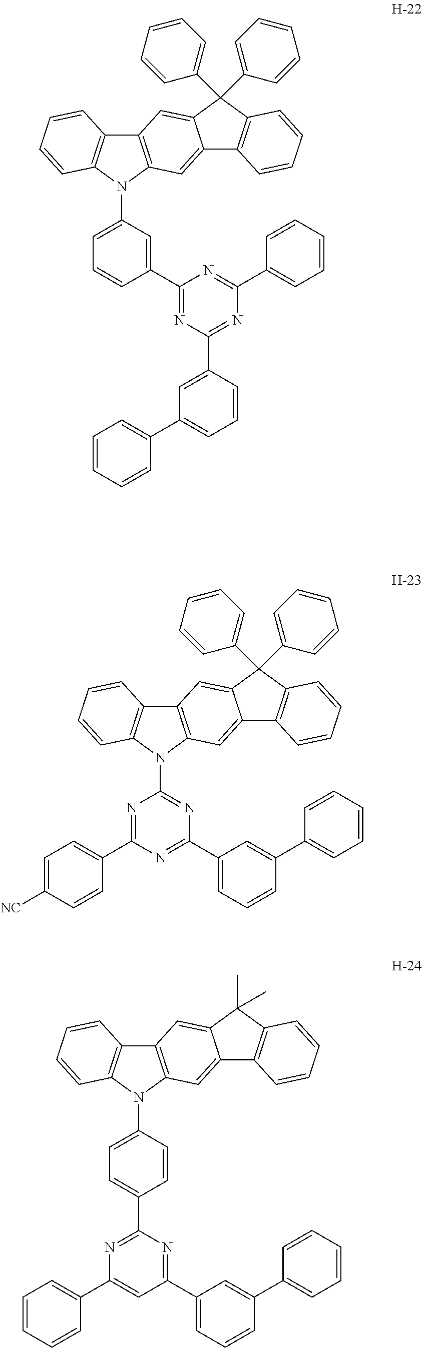 [SMILES: c1ccc(-c2cccc(-c3nc(-c4ccccc4)nc(-c4cccc(-n5c6ccccc6c6cc7c(cc65)-c5ccccc5C7(c5ccccc5)c5ccccc5)c4)n3)c2)cc1 ; N#Cc1ccc(-c2nc(-c3cccc(-c4ccccc4)c3)nc(-n3c4ccccc4c4cc5c(cc43)-c3ccccc3C5(c3ccccc3)c3ccccc3)n2)cc1 ; CC1(C)c2ccccc2-c2cc3c(cc21)c1ccccc1n3-c1ccc(-c2nc(-c3ccccc3)cc(-c3cccc(-c4ccccc4)c3)n2)cc1]

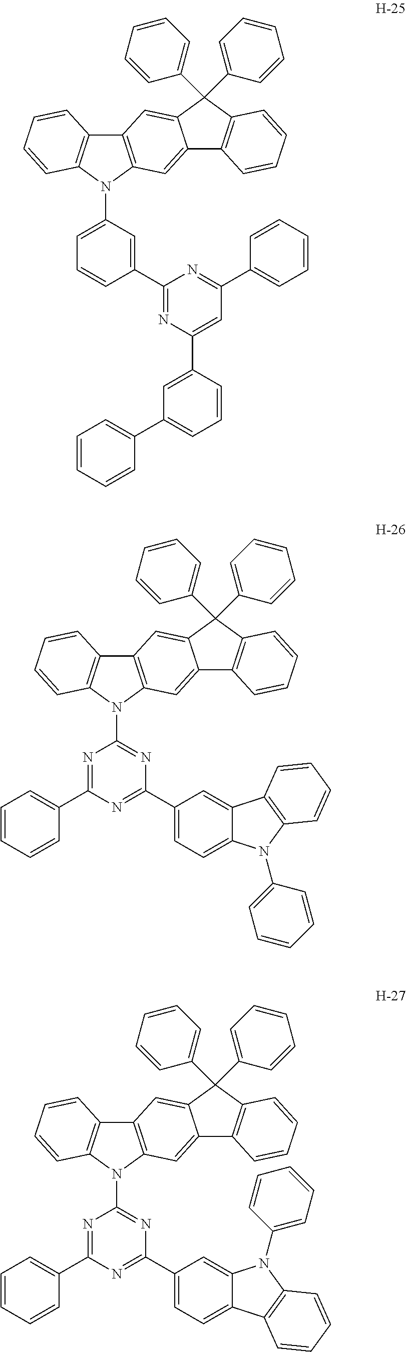 [SMILES: c1ccc(-c2cccc(-c3cc(-c4ccccc4)nc(-c4cccc(-n5c6ccccc6c6cc7c(cc65)-c5ccccc5C7(c5ccccc5)c5ccccc5)c4)n3)c2)cc1 ; c1ccc(-c2nc(-c3ccc4c(c3)c3ccccc3n4-c3ccccc3)nc(-n3c4ccccc4c4cc5c(cc43)-c3ccccc3C5(c3ccccc3)c3ccccc3)n2)cc1 ; c1ccc(-c2nc(-c3ccc4c5ccccc5n(-c5ccccc5)c4c3)nc(-n3c4ccccc4c4cc5c(cc43)-c3ccccc3C5(c3ccccc3)c3ccccc3)n2)cc1]

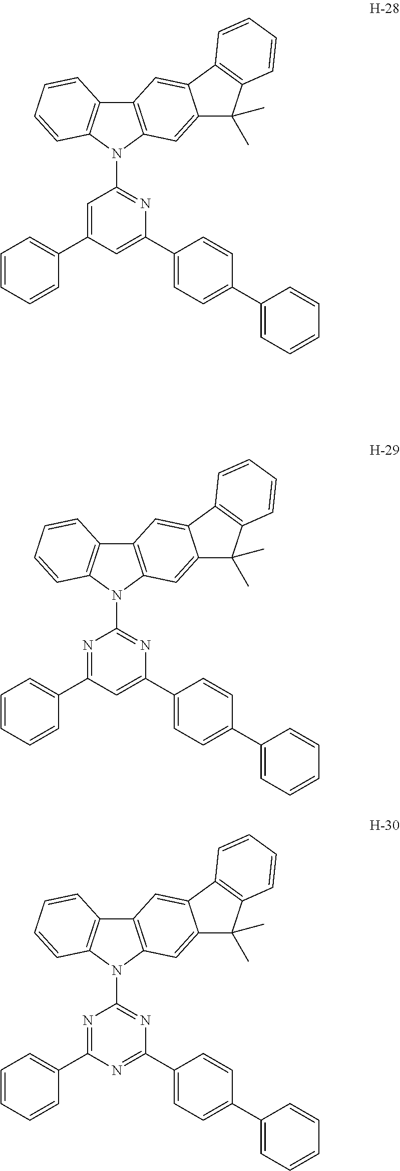 [SMILES: CC1(C)c2ccccc2-c2cc3c4ccccc4n(-c4cc(-c5ccccc5)cc(-c5ccc(-c6ccccc6)cc5)n4)c3cc21 ; CC1(C)c2ccccc2-c2cc3c4ccccc4n(-c4nc(-c5ccccc5)cc(-c5ccc(-c6ccccc6)cc5)n4)c3cc21 ; CC1(C)c2ccccc2-c2cc3c4ccccc4n(-c4nc(-c5ccccc5)nc(-c5ccc(-c6ccccc6)cc5)n4)c3cc21]

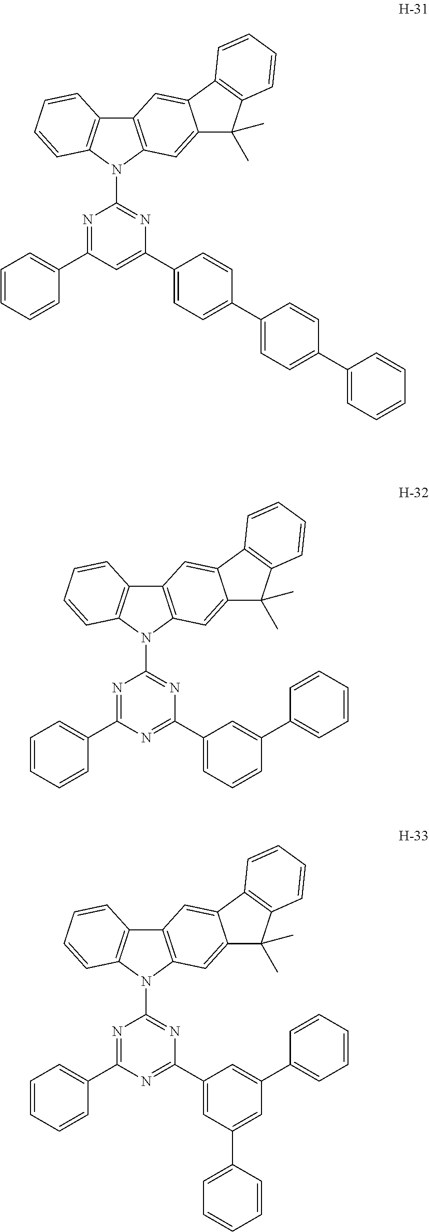 [SMILES: CC1(C)c2ccccc2-c2cc3c4ccccc4n(-c4nc(-c5ccccc5)cc(-c5ccc(-c6ccc(-c7ccccc7)cc6)cc5)n4)c3cc21 ; CC1(C)c2ccccc2-c2cc3c4ccccc4n(-c4nc(-c5ccccc5)nc(-c5cccc(-c6ccccc6)c5)n4)c3cc21 ; CC1(C)c2ccccc2-c2cc3c4ccccc4n(-c4nc(-c5ccccc5)nc(-c5cc(-c6ccccc6)cc(-c6ccccc6)c5)n4)c3cc21]

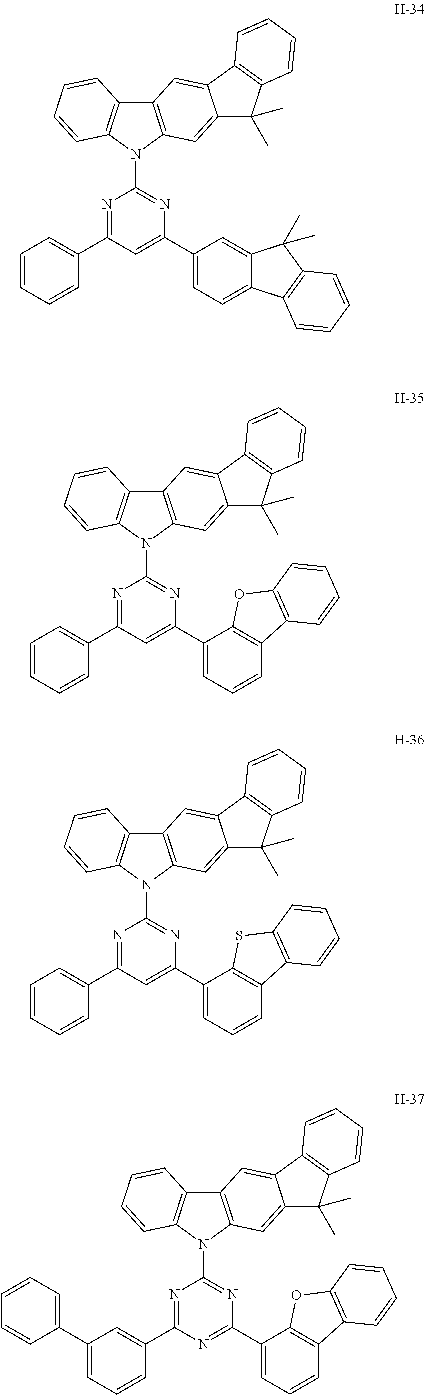 [SMILES: CC1(C)c2ccccc2-c2ccc(-c3cc(-c4ccccc4)nc(-n4c5ccccc5c5cc6c(cc54)C(C)(C)c4ccccc4-6)n3)cc21 ; CC1(C)c2ccccc2-c2cc3c4ccccc4n(-c4nc(-c5ccccc5)cc(-c5cccc6c5oc5ccccc56)n4)c3cc21 ; CC1(C)c2ccccc2-c2cc3c4ccccc4n(-c4nc(-c5ccccc5)cc(-c5cccc6c5sc5ccccc56)n4)c3cc21 ; CC1(C)c2ccccc2-c2cc3c4ccccc4n(-c4nc(-c5cccc(-c6ccccc6)c5)nc(-c5cccc6c5oc5ccccc56)n4)c3cc21]

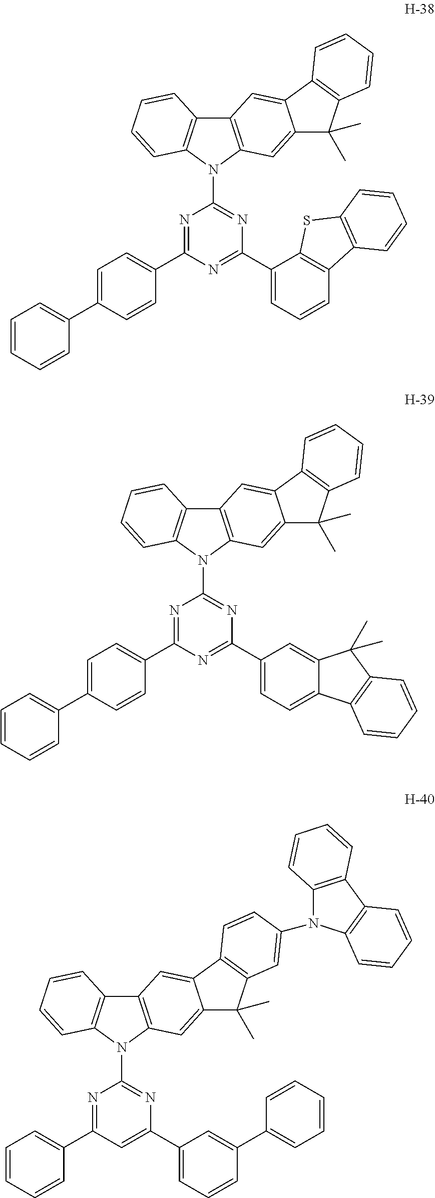 [SMILES: CC1(C)c2ccccc2-c2cc3c4ccccc4n(-c4nc(-c5ccc(-c6ccccc6)cc5)nc(-c5cccc6c5sc5ccccc56)n4)c3cc21 ; CC1(C)c2ccccc2-c2ccc(-c3nc(-c4ccc(-c5ccccc5)cc4)nc(-n4c5ccccc5c5cc6c(cc54)C(C)(C)c4ccccc4-6)n3)cc21 ; CC1(C)c2cc(-n3c4ccccc4c4ccccc43)ccc2-c2cc3c4ccccc4n(-c4nc(-c5ccccc5)cc(-c5cccc(-c6ccccc6)c5)n4)c3cc21]

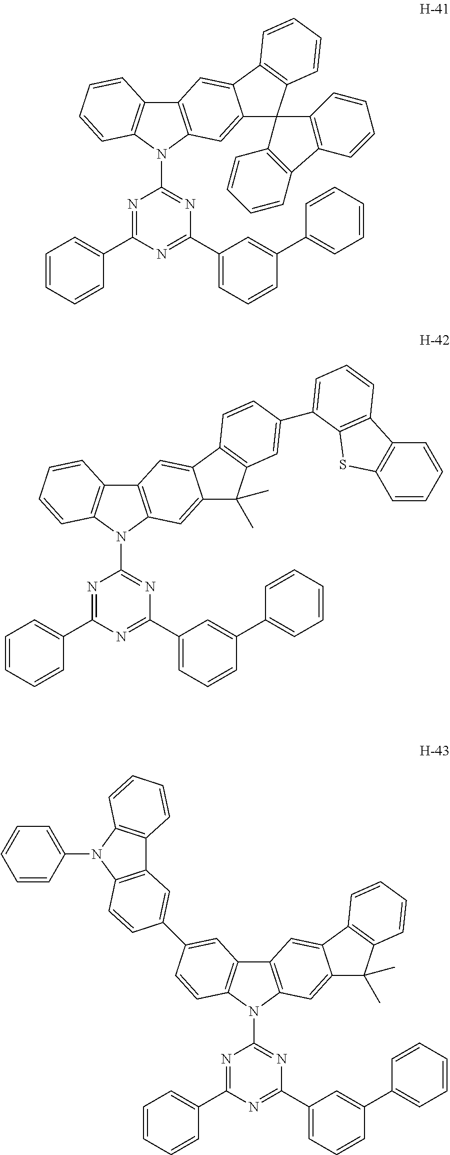 [SMILES: c1ccc(-c2cccc(-c3nc(-c4ccccc4)nc(-n4c5ccccc5c5cc6c(cc54)C4(c5ccccc5-c5ccccc54)c4ccccc4-6)n3)c2)cc1 ; CC1(C)c2cc(-c3cccc4c3sc3ccccc34)ccc2-c2cc3c4ccccc4n(-c4nc(-c5ccccc5)nc(-c5cccc(-c6ccccc6)c5)n4)c3cc21 ; CC1(C)c2ccccc2-c2cc3c4cc(-c5ccc6c(c5)c5ccccc5n6-c5ccccc5)ccc4n(-c4nc(-c5ccccc5)nc(-c5cccc(-c6ccccc6)c5)n4)c3cc21]

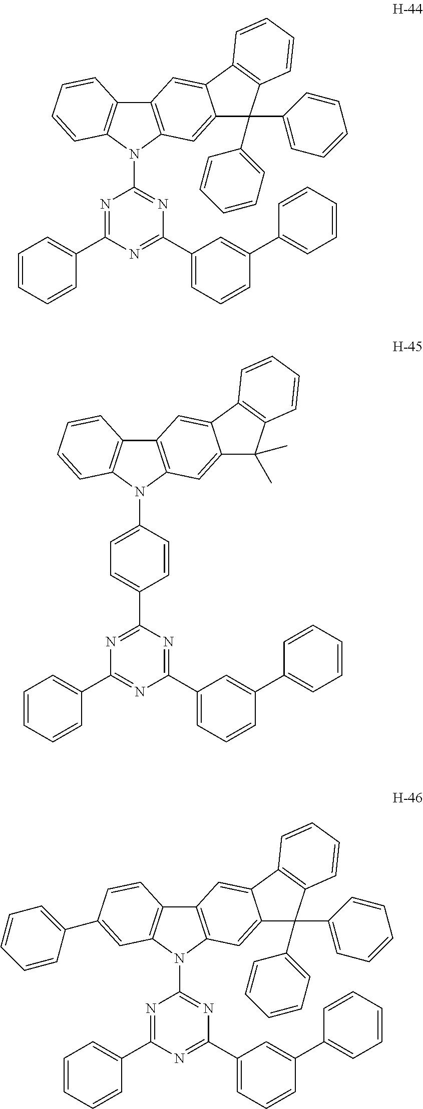 [SMILES: c1ccc(-c2cccc(-c3nc(-c4ccccc4)nc(-n4c5ccccc5c5cc6c(cc54)C(c4ccccc4)(c4ccccc4)c4ccccc4-6)n3)c2)cc1 ; CC1(C)c2ccccc2-c2cc3c4ccccc4n(-c4ccc(-c5nc(-c6ccccc6)nc(-c6cccc(-c7ccccc7)c6)n5)cc4)c3cc21 ; c1ccc(-c2cccc(-c3nc(-c4ccccc4)nc(-n4c5cc(-c6ccccc6)ccc5c5cc6c(cc54)C(c4ccccc4)(c4ccccc4)c4ccccc4-6)n3)c2)cc1]

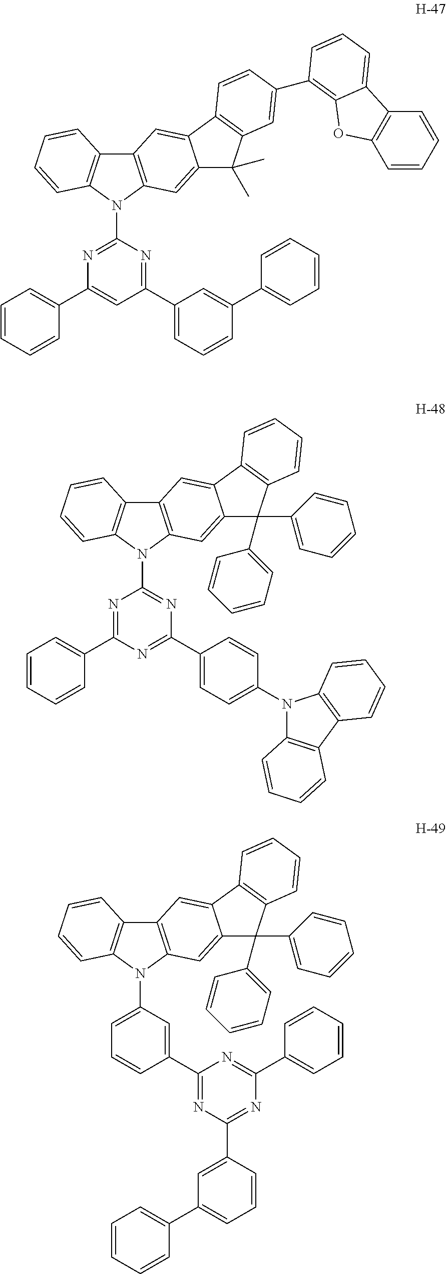

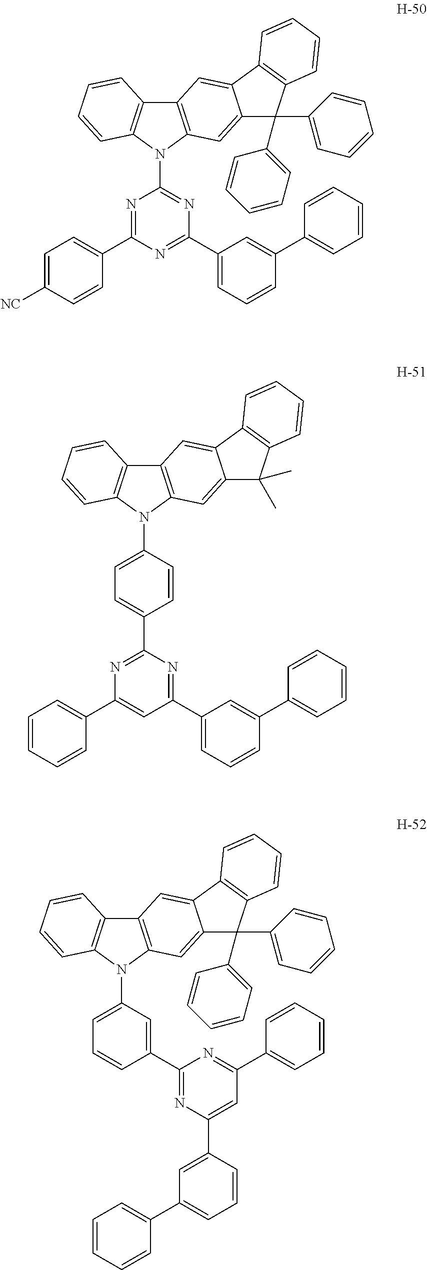 [SMILES: N#Cc1ccc(-c2nc(-c3cccc(-c4ccccc4)c3)nc(-n3c4ccccc4c4cc5c(cc43)C(c3ccccc3)(c3ccccc3)c3ccccc3-5)n2)cc1 ; CC1(C)c2ccccc2-c2cc3c4ccccc4n(-c4ccc(-c5nc(-c6ccccc6)cc(-c6cccc(-c7ccccc7)c6)n5)cc4)c3cc21 ; c1ccc(-c2cccc(-c3cc(-c4ccccc4)nc(-c4cccc(-n5c6ccccc6c6cc7c(cc65)C(c5ccccc5)(c5ccccc5)c5ccccc5-7)c4)n3)c2)cc1]

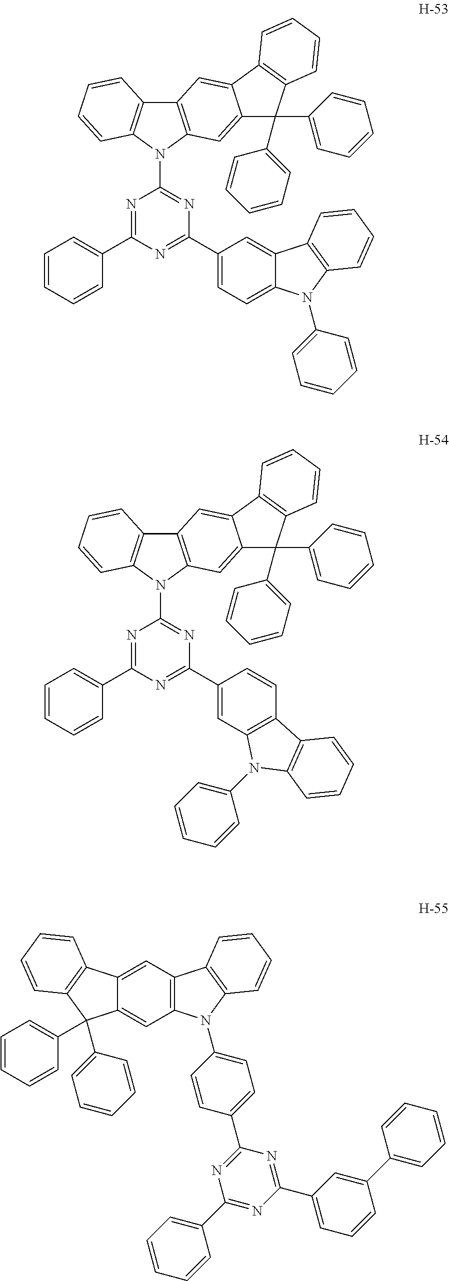 [SMILES: c1ccc(-c2nc(-c3ccc4c(c3)c3ccccc3n4-c3ccccc3)nc(-n3c4ccccc4c4cc5c(cc43)C(c3ccccc3)(c3ccccc3)c3ccccc3-5)n2)cc1 ; c1ccc(-c2nc(-c3ccc4c5ccccc5n(-c5ccccc5)c4c3)nc(-n3c4ccccc4c4cc5c(cc43)C(c3ccccc3)(c3ccccc3)c3ccccc3-5)n2)cc1 ; c1ccc(-c2cccc(-c3nc(-c4ccccc4)nc(-c4ccc(-n5c6ccccc6c6cc7c(cc65)C(c5ccccc5)(c5ccccc5)c5ccccc5-7)cc4)n3)c2)cc1]

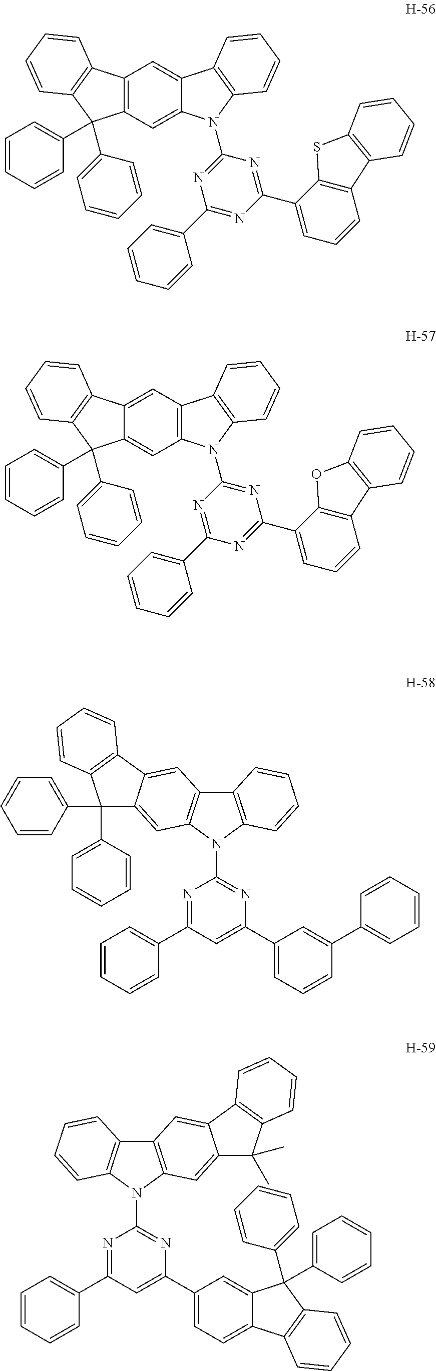 [SMILES: c1ccc(-c2nc(-c3cccc4c3sc3ccccc34)nc(-n3c4ccccc4c4cc5c(cc43)C(c3ccccc3)(c3ccccc3)c3ccccc3-5)n2)cc1 ; c1ccc(-c2nc(-c3cccc4c3oc3ccccc34)nc(-n3c4ccccc4c4cc5c(cc43)C(c3ccccc3)(c3ccccc3)c3ccccc3-5)n2)cc1 ; c1ccc(-c2cccc(-c3cc(-c4ccccc4)nc(-n4c5ccccc5c5cc6c(cc54)C(c4ccccc4)(c4ccccc4)c4ccccc4-6)n3)c2)cc1 ; CC1(C)c2ccccc2-c2cc3c4ccccc4n(-c4nc(-c5ccccc5)cc(-c5ccc6c(c5)C(c5ccccc5)(c5ccccc5)c5ccccc5-6)n4)c3cc21]

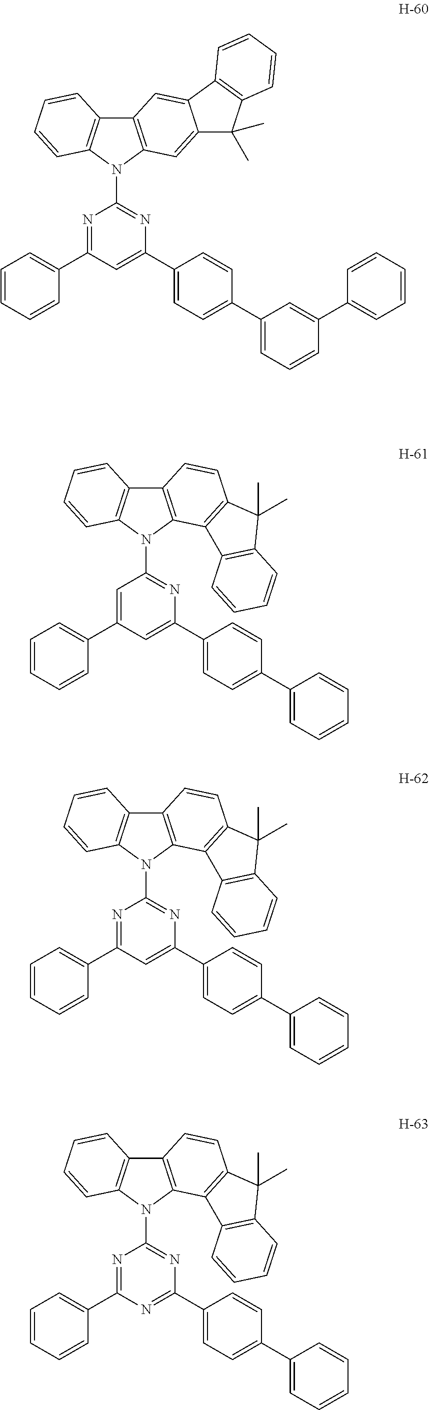 [SMILES: CC1(C)c2ccccc2-c2cc3c4ccccc4n(-c4nc(-c5ccccc5)cc(-c5ccc(-c6cccc(-c7ccccc7)c6)cc5)n4)c3cc21 ; CC1(C)c2ccccc2-c2c1ccc1c3ccccc3n(-c3cc(-c4ccccc4)cc(-c4ccc(-c5ccccc5)cc4)n3)c21 ; CC1(C)c2ccccc2-c2c1ccc1c3ccccc3n(-c3nc(-c4ccccc4)cc(-c4ccc(-c5ccccc5)cc4)n3)c21 ; CC1(C)c2ccccc2-c2c1ccc1c3ccccc3n(-c3nc(-c4ccccc4)nc(-c4ccc(-c5ccccc5)cc4)n3)c21]

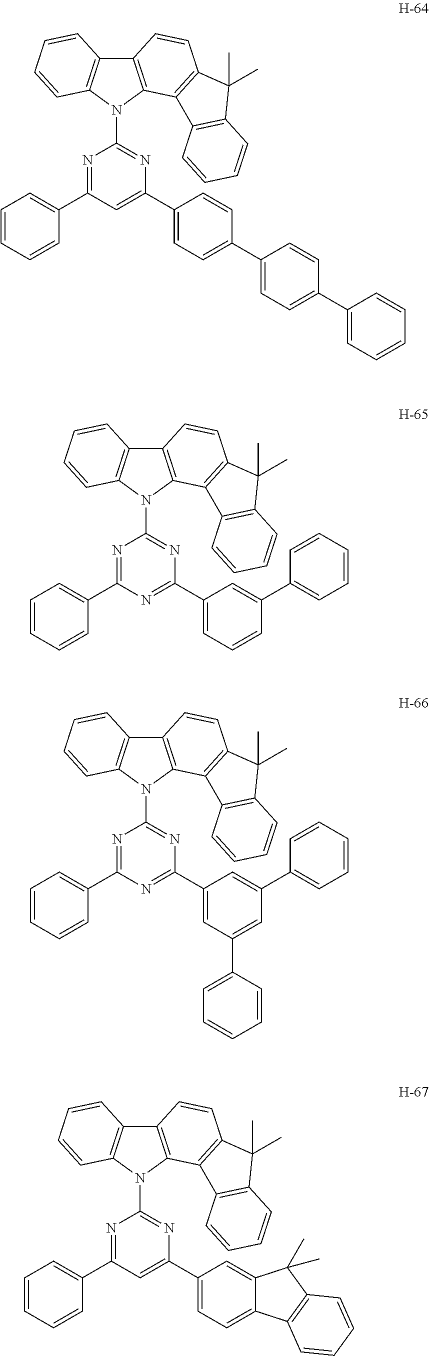 [SMILES: CC1(C)c2ccccc2-c2c1ccc1c3ccccc3n(-c3nc(-c4ccccc4)cc(-c4ccc(-c5ccc(-c6ccccc6)cc5)cc4)n3)c21 ; CC1(C)c2ccccc2-c2c1ccc1c3ccccc3n(-c3nc(-c4ccccc4)nc(-c4cccc(-c5ccccc5)c4)n3)c21 ; CC1(C)c2ccccc2-c2c1ccc1c3ccccc3n(-c3nc(-c4ccccc4)nc(-c4cc(-c5ccccc5)cc(-c5ccccc5)c4)n3)c21 ; CC1(C)c2ccccc2-c2ccc(-c3cc(-c4ccccc4)nc(-n4c5ccccc5c5ccc6c(c54)-c4ccccc4C6(C)C)n3)cc21]

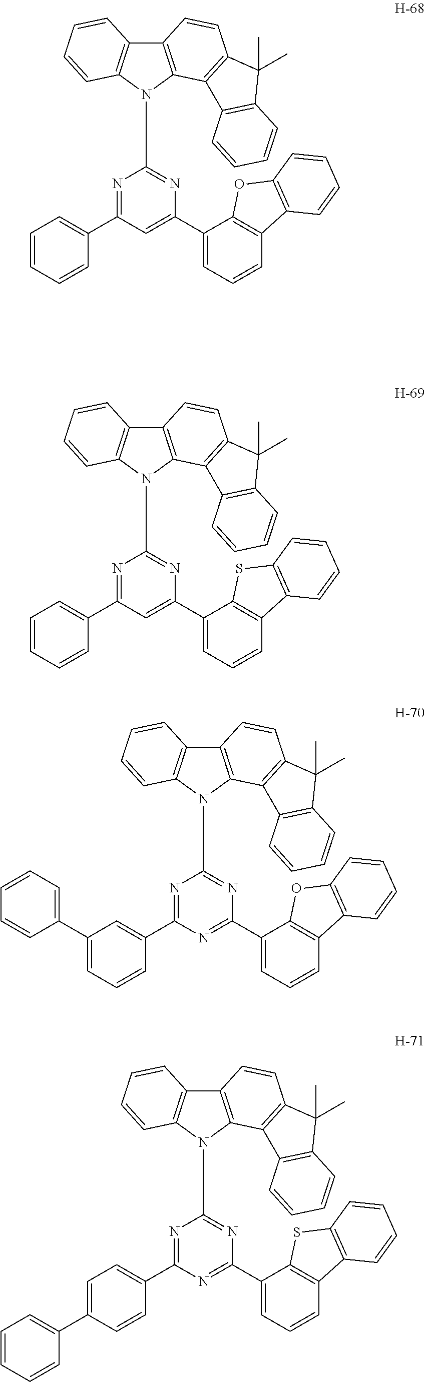 [SMILES: CC1(C)c2ccccc2-c2c1ccc1c3ccccc3n(-c3nc(-c4ccccc4)cc(-c4cccc5c4oc4ccccc45)n3)c21 ; CC1(C)c2ccccc2-c2c1ccc1c3ccccc3n(-c3nc(-c4ccccc4)cc(-c4cccc5c4sc4ccccc45)n3)c21 ; CC1(C)c2ccccc2-c2c1ccc1c3ccccc3n(-c3nc(-c4cccc(-c5ccccc5)c4)nc(-c4cccc5c4oc4ccccc45)n3)c21 ; CC1(C)c2ccccc2-c2c1ccc1c3ccccc3n(-c3nc(-c4ccc(-c5ccccc5)cc4)nc(-c4cccc5c4sc4ccccc45)n3)c21]

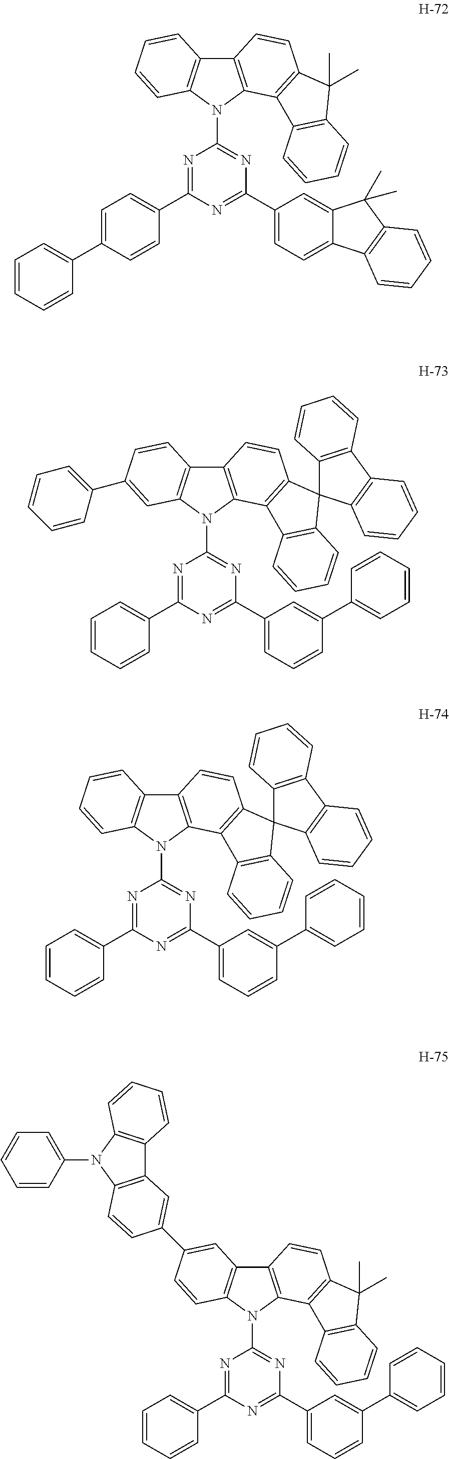 [SMILES: CC1(C)c2ccccc2-c2ccc(-c3nc(-c4ccc(-c5ccccc5)cc4)nc(-n4c5ccccc5c5ccc6c(c54)-c4ccccc4C6(C)C)n3)cc21 ; c1ccc(-c2cccc(-c3nc(-c4ccccc4)nc(-n4c5cc(-c6ccccc6)ccc5c5ccc6c(c54)-c4ccccc4C64c5ccccc5-c5ccccc54)n3)c2)cc1 ; c1ccc(-c2cccc(-c3nc(-c4ccccc4)nc(-n4c5ccccc5c5ccc6c(c54)-c4ccccc4C64c5ccccc5-c5ccccc54)n3)c2)cc1 ; CC1(C)c2ccccc2-c2c1ccc1c3cc(-c4ccc5c(c4)c4ccccc4n5-c4ccccc4)ccc3n(-c3nc(-c4ccccc4)nc(-c4cccc(-c5ccccc5)c4)n3)c21]

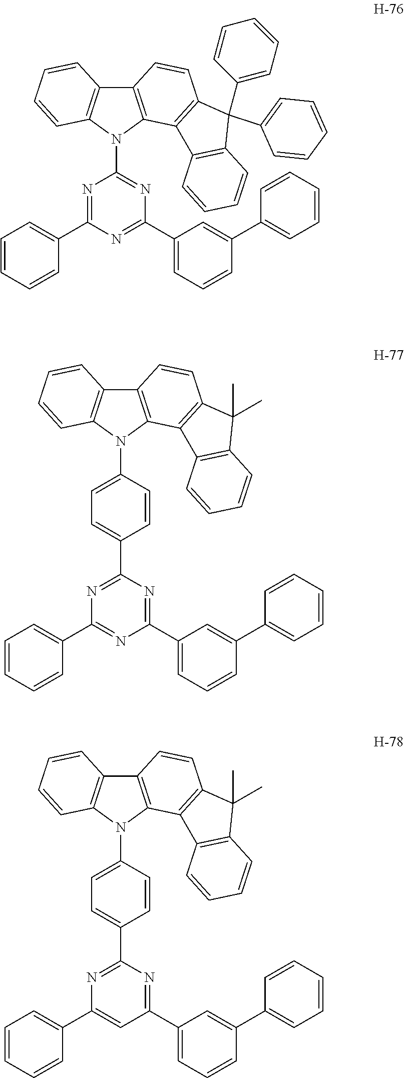 [SMILES: c1ccc(-c2cccc(-c3nc(-c4ccccc4)nc(-n4c5ccccc5c5ccc6c(c54)-c4ccccc4C6(c4ccccc4)c4ccccc4)n3)c2)cc1 ; CC1(C)c2ccccc2-c2c1ccc1c3ccccc3n(-c3ccc(-c4nc(-c5ccccc5)nc(-c5cccc(-c6ccccc6)c5)n4)cc3)c21 ; CC1(C)c2ccccc2-c2c1ccc1c3ccccc3n(-c3ccc(-c4nc(-c5ccccc5)cc(-c5cccc(-c6ccccc6)c5)n4)cc3)c21]

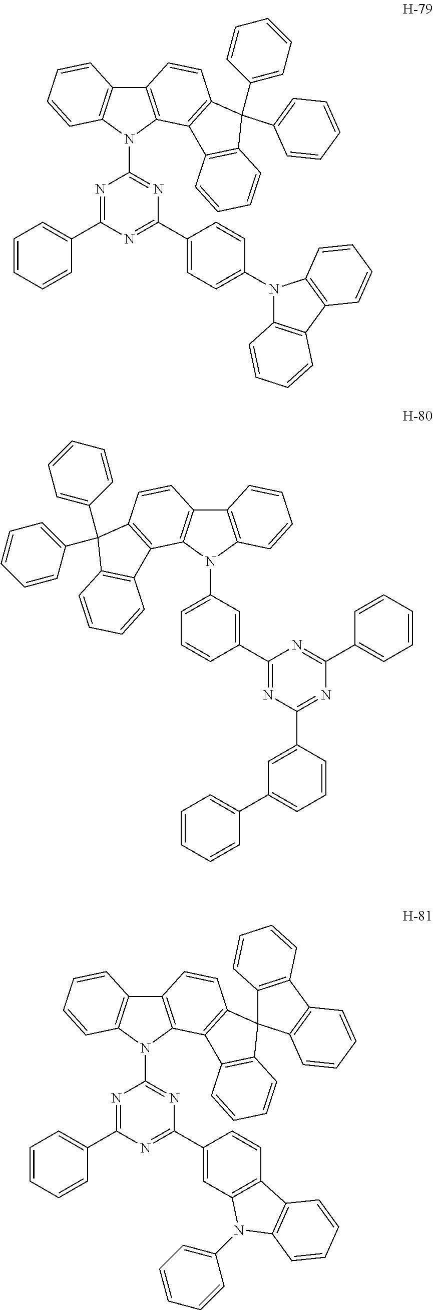 [SMILES: c1ccc(-c2nc(-c3ccc(-n4c5ccccc5c5ccccc54)cc3)nc(-n3c4ccccc4c4ccc5c(c43)-c3ccccc3C5(c3ccccc3)c3ccccc3)n2)cc1 ; c1ccc(-c2cccc(-c3nc(-c4ccccc4)nc(-c4cccc(-n5c6ccccc6c6ccc7c(c65)-c5ccccc5C7(c5ccccc5)c5ccccc5)c4)n3)c2)cc1 ; c1ccc(-c2nc(-c3ccc4c5ccccc5n(-c5ccccc5)c4c3)nc(-n3c4ccccc4c4ccc5c(c43)-c3ccccc3C53c4ccccc4-c4ccccc43)n2)cc1]

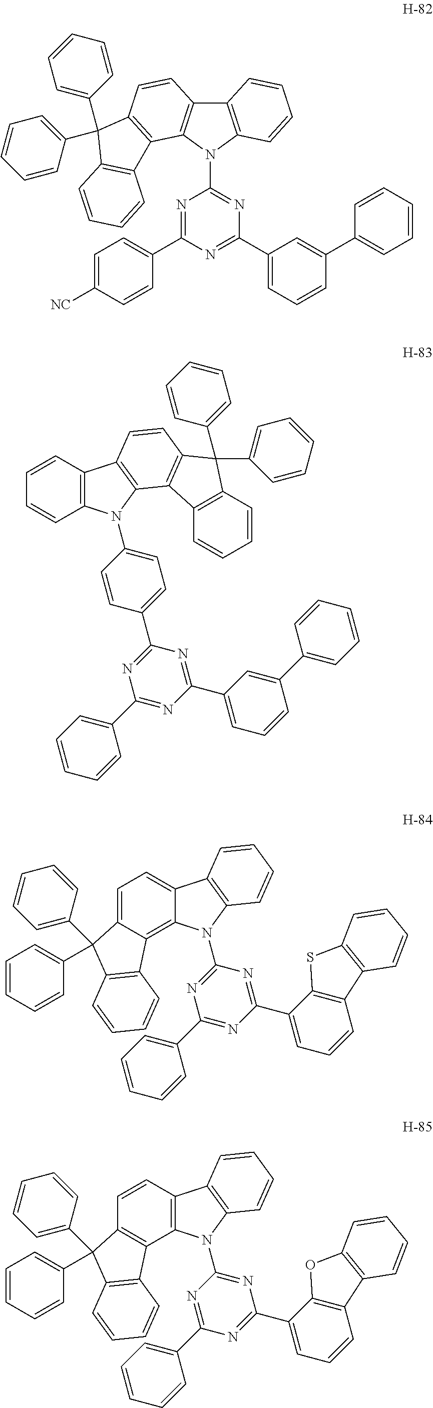 [SMILES: N#Cc1ccc(-c2nc(-c3cccc(-c4ccccc4)c3)nc(-n3c4ccccc4c4ccc5c(c43)-c3ccccc3C5(c3ccccc3)c3ccccc3)n2)cc1 ; c1ccc(-c2cccc(-c3nc(-c4ccccc4)nc(-c4ccc(-n5c6ccccc6c6ccc7c(c65)-c5ccccc5C7(c5ccccc5)c5ccccc5)cc4)n3)c2)cc1 ; c1ccc(-c2nc(-c3cccc4c3sc3ccccc34)nc(-n3c4ccccc4c4ccc5c(c43)-c3ccccc3C5(c3ccccc3)c3ccccc3)n2)cc1 ; c1ccc(-c2nc(-c3cccc4c3oc3ccccc34)nc(-n3c4ccccc4c4ccc5c(c43)-c3ccccc3C5(c3ccccc3)c3ccccc3)n2)cc1]

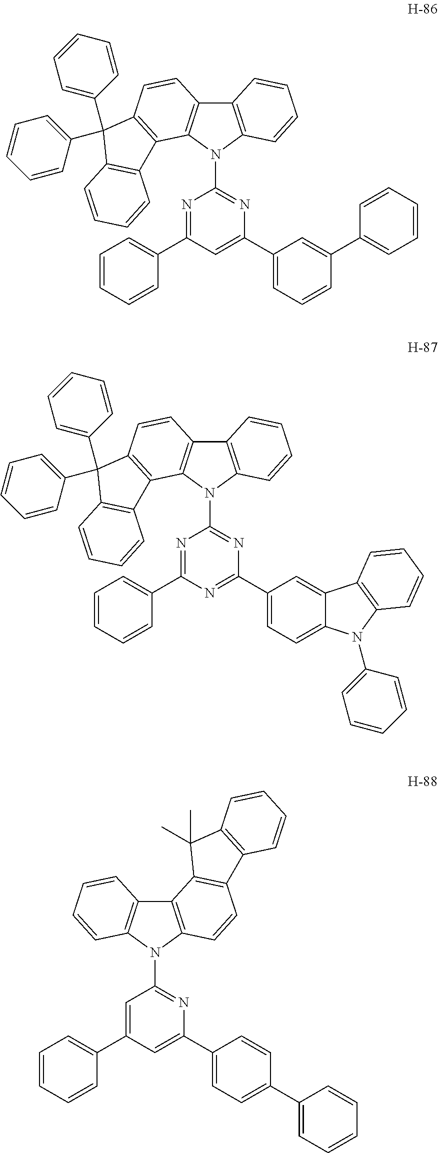 [SMILES: c1ccc(-c2cccc(-c3cc(-c4ccccc4)nc(-n4c5ccccc5c5ccc6c(c54)-c4ccccc4C6(c4ccccc4)c4ccccc4)n3)c2)cc1 ; c1ccc(-c2nc(-c3ccc4c(c3)c3ccccc3n4-c3ccccc3)nc(-n3c4ccccc4c4ccc5c(c43)-c3ccccc3C5(c3ccccc3)c3ccccc3)n2)cc1 ; CC1(C)c2ccccc2-c2ccc3c(c21)c1ccccc1n3-c1cc(-c2ccccc2)cc(-c2ccc(-c3ccccc3)cc2)n1]

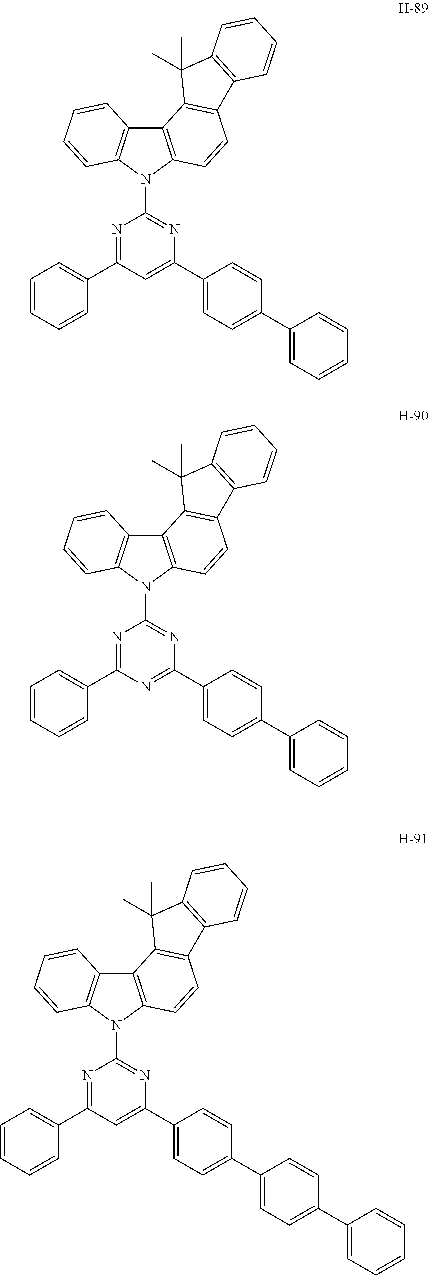 [SMILES: CC1(C)c2ccccc2-c2ccc3c(c21)c1ccccc1n3-c1nc(-c2ccccc2)cc(-c2ccc(-c3ccccc3)cc2)n1 ; CC1(C)c2ccccc2-c2ccc3c(c21)c1ccccc1n3-c1nc(-c2ccccc2)nc(-c2ccc(-c3ccccc3)cc2)n1 ; CC1(C)c2ccccc2-c2ccc3c(c21)c1ccccc1n3-c1nc(-c2ccccc2)cc(-c2ccc(-c3ccc(-c4ccccc4)cc3)cc2)n1]

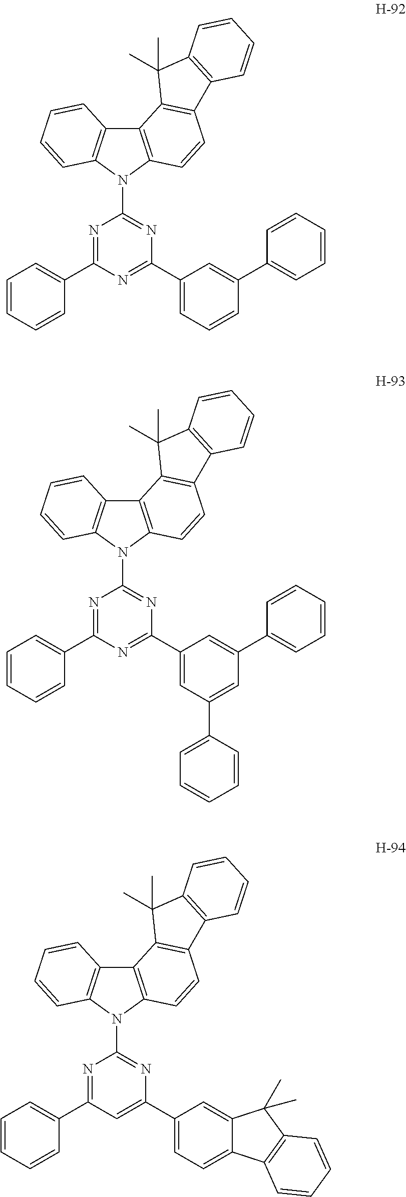 [SMILES: CC1(C)c2ccccc2-c2ccc3c(c21)c1ccccc1n3-c1nc(-c2ccccc2)nc(-c2cccc(-c3ccccc3)c2)n1 ; CC1(C)c2ccccc2-c2ccc3c(c21)c1ccccc1n3-c1nc(-c2ccccc2)nc(-c2cc(-c3ccccc3)cc(-c3ccccc3)c2)n1 ; CC1(C)c2ccccc2-c2ccc(-c3cc(-c4ccccc4)nc(-n4c5ccccc5c5c6c(ccc54)-c4ccccc4C6(C)C)n3)cc21]

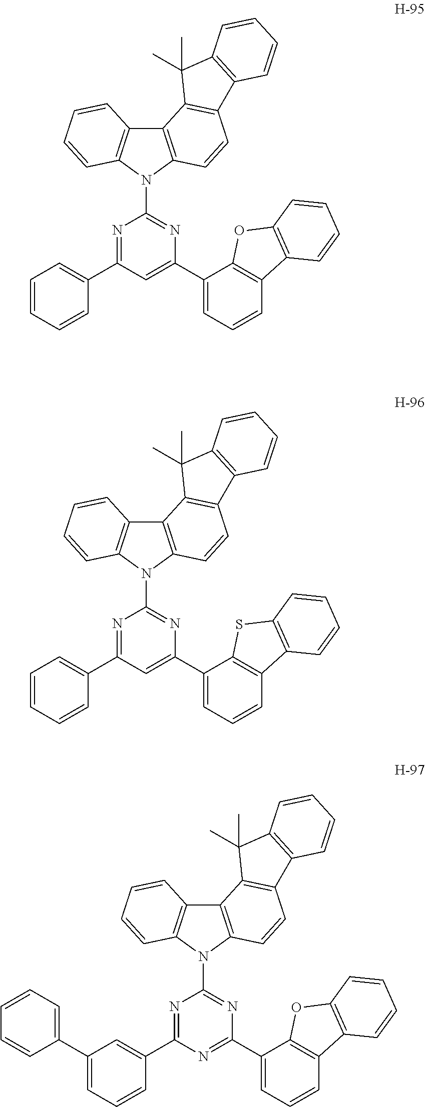 [SMILES: CC1(C)c2ccccc2-c2ccc3c(c21)c1ccccc1n3-c1nc(-c2ccccc2)cc(-c2cccc3c2oc2ccccc23)n1 ; CC1(C)c2ccccc2-c2ccc3c(c21)c1ccccc1n3-c1nc(-c2ccccc2)cc(-c2cccc3c2sc2ccccc23)n1 ; CC1(C)c2ccccc2-c2ccc3c(c21)c1ccccc1n3-c1nc(-c2cccc(-c3ccccc3)c2)nc(-c2cccc3c2oc2ccccc23)n1]

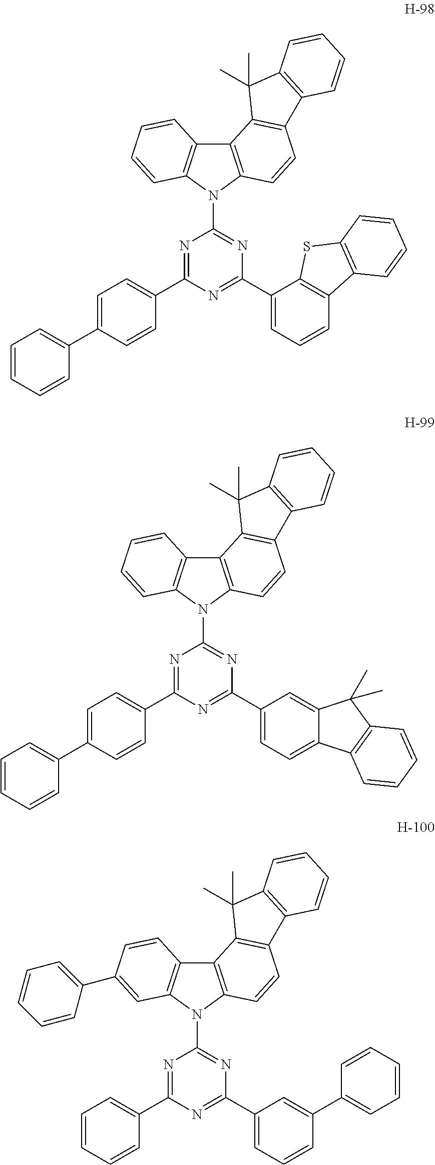 [SMILES: CC1(C)c2ccccc2-c2ccc3c(c21)c1ccccc1n3-c1nc(-c2ccc(-c3ccccc3)cc2)nc(-c2cccc3c2sc2ccccc23)n1 ; CC1(C)c2ccccc2-c2ccc(-c3nc(-c4ccc(-c5ccccc5)cc4)nc(-n4c5ccccc5c5c6c(ccc54)-c4ccccc4C6(C)C)n3)cc21 ; CC1(C)c2ccccc2-c2ccc3c(c21)c1ccc(-c2ccccc2)cc1n3-c1nc(-c2ccccc2)nc(-c2cccc(-c3ccccc3)c2)n1]

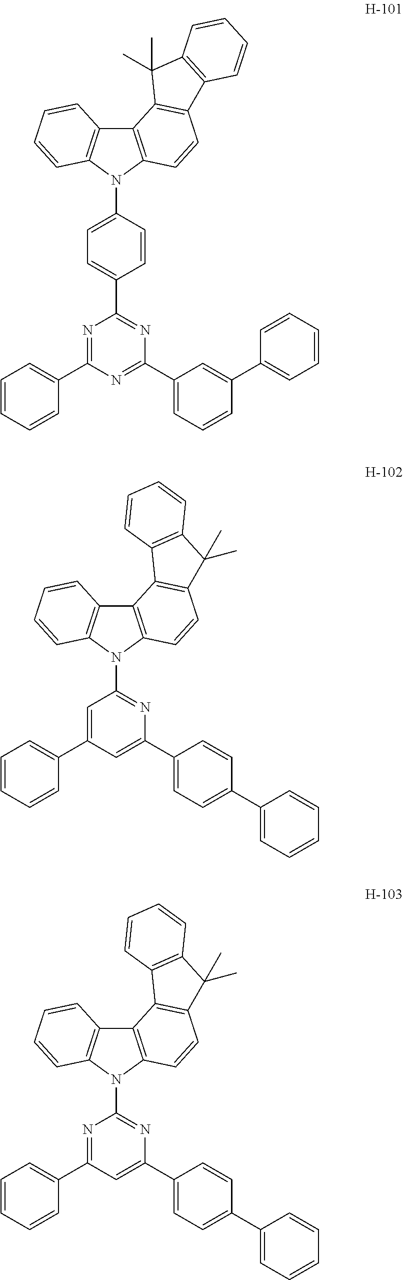 [SMILES: CC1(C)c2ccccc2-c2ccc3c(c21)c1ccccc1n3-c1ccc(-c2nc(-c3ccccc3)nc(-c3cccc(-c4ccccc4)c3)n2)cc1 ; CC1(C)c2ccccc2-c2c1ccc1c2c2ccccc2n1-c1cc(-c2ccccc2)cc(-c2ccc(-c3ccccc3)cc2)n1 ; CC1(C)c2ccccc2-c2c1ccc1c2c2ccccc2n1-c1nc(-c2ccccc2)cc(-c2ccc(-c3ccccc3)cc2)n1]

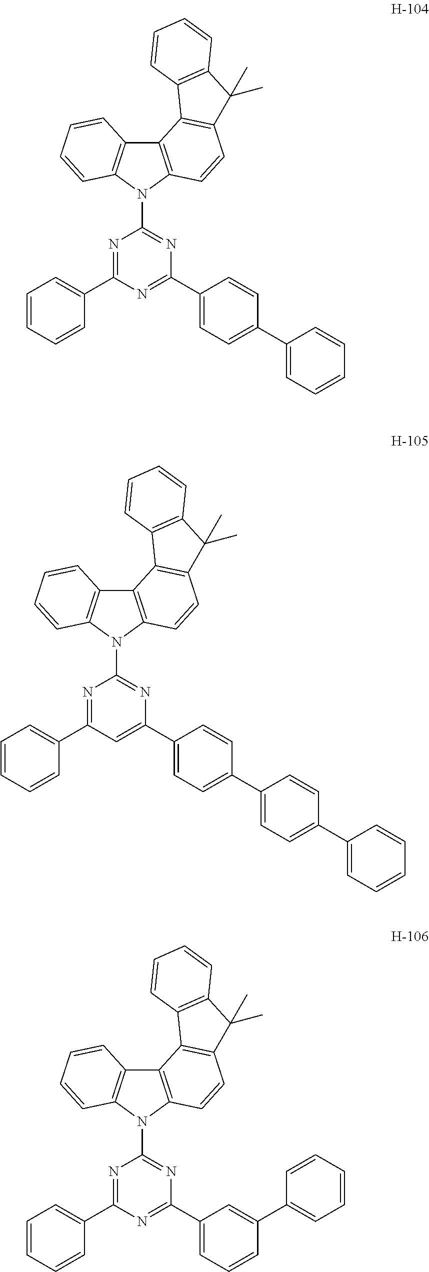 [SMILES: CC1(C)c2ccccc2-c2c1ccc1c2c2ccccc2n1-c1nc(-c2ccccc2)nc(-c2ccc(-c3ccccc3)cc2)n1 ; CC1(C)c2ccccc2-c2c1ccc1c2c2ccccc2n1-c1nc(-c2ccccc2)cc(-c2ccc(-c3ccc(-c4ccccc4)cc3)cc2)n1 ; CC1(C)c2ccccc2-c2c1ccc1c2c2ccccc2n1-c1nc(-c2ccccc2)nc(-c2cccc(-c3ccccc3)c2)n1]

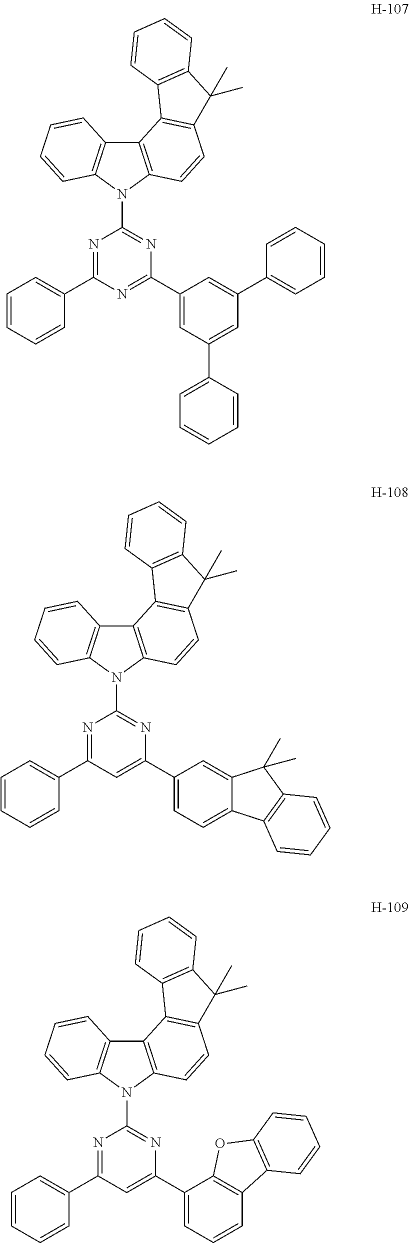 [SMILES: CC1(C)c2ccccc2-c2c1ccc1c2c2ccccc2n1-c1nc(-c2ccccc2)nc(-c2cc(-c3ccccc3)cc(-c3ccccc3)c2)n1 ; CC1(C)c2ccccc2-c2ccc(-c3cc(-c4ccccc4)nc(-n4c5ccccc5c5c6c(ccc54)C(C)(C)c4ccccc4-6)n3)cc21 ; CC1(C)c2ccccc2-c2c1ccc1c2c2ccccc2n1-c1nc(-c2ccccc2)cc(-c2cccc3c2oc2ccccc23)n1]

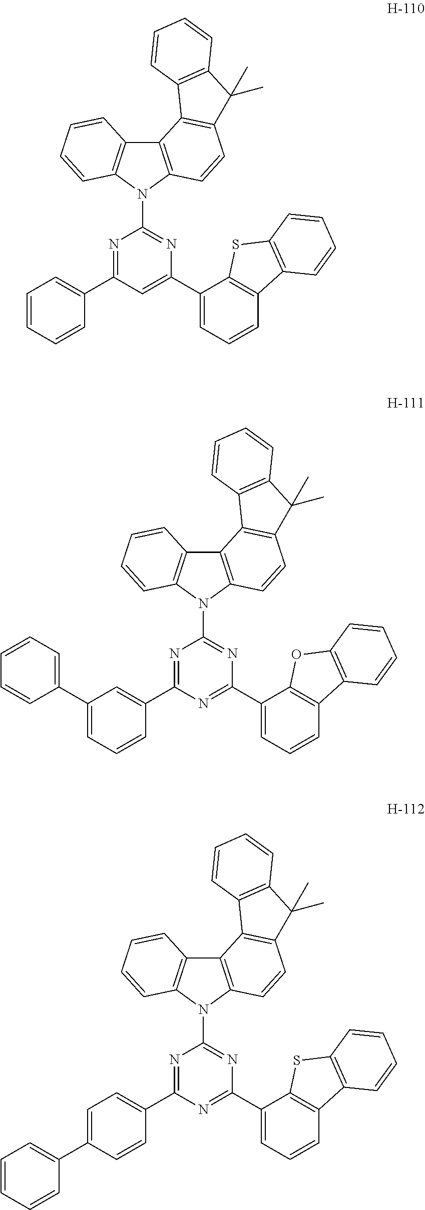 [SMILES: CC1(C)c2ccccc2-c2c1ccc1c2c2ccccc2n1-c1nc(-c2ccccc2)cc(-c2cccc3c2sc2ccccc23)n1 ; CC1(C)c2ccccc2-c2c1ccc1c2c2ccccc2n1-c1nc(-c2cccc(-c3ccccc3)c2)nc(-c2cccc3c2oc2ccccc23)n1 ; CC1(C)c2ccccc2-c2c1ccc1c2c2ccccc2n1-c1nc(-c2ccc(-c3ccccc3)cc2)nc(-c2cccc3c2sc2ccccc23)n1]

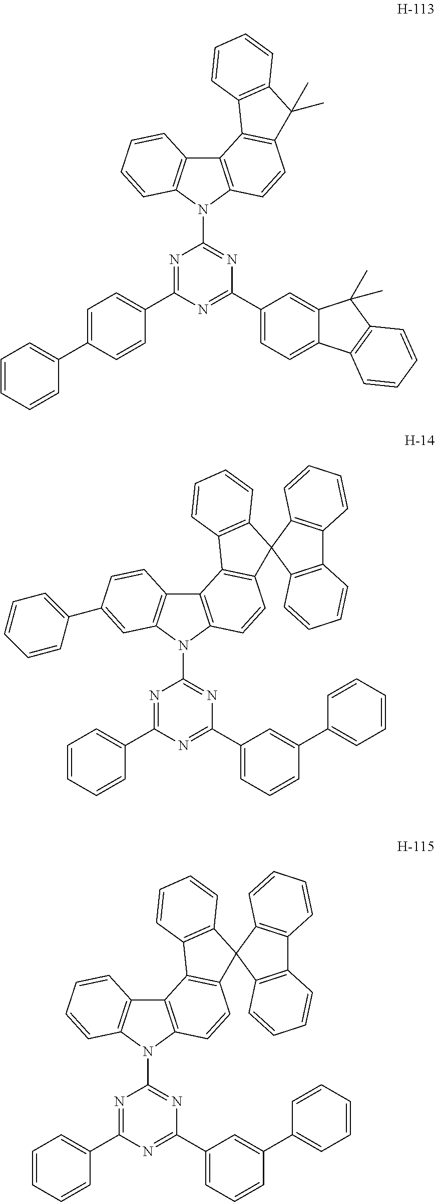 [SMILES: CC1(C)c2ccccc2-c2ccc(-c3nc(-c4ccc(-c5ccccc5)cc4)nc(-n4c5ccccc5c5c6c(ccc54)C(C)(C)c4ccccc4-6)n3)cc21 ; c1ccc(-c2cccc(-c3nc(-c4ccccc4)nc(-n4c5cc(-c6ccccc6)ccc5c5c6c(ccc54)C4(c5ccccc5-c5ccccc54)c4ccccc4-6)n3)c2)cc1 ; c1ccc(-c2cccc(-c3nc(-c4ccccc4)nc(-n4c5ccccc5c5c6c(ccc54)C4(c5ccccc5-c5ccccc54)c4ccccc4-6)n3)c2)cc1]

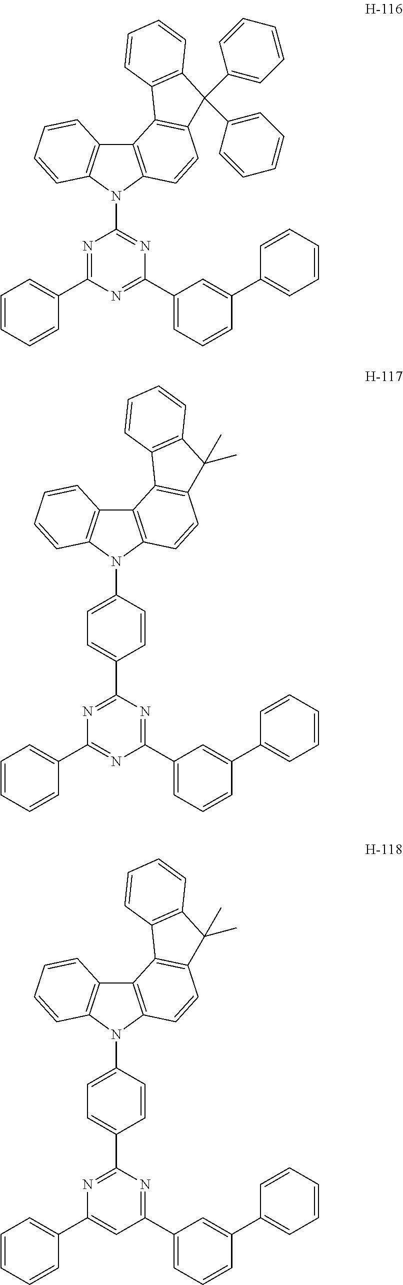 [SMILES: c1ccc(-c2cccc(-c3nc(-c4ccccc4)nc(-n4c5ccccc5c5c6c(ccc54)C(c4ccccc4)(c4ccccc4)c4ccccc4-6)n3)c2)cc1 ; CC1(C)c2ccccc2-c2c1ccc1c2c2ccccc2n1-c1ccc(-c2nc(-c3ccccc3)nc(-c3cccc(-c4ccccc4)c3)n2)cc1 ; CC1(C)c2ccccc2-c2c1ccc1c2c2ccccc2n1-c1ccc(-c2nc(-c3ccccc3)cc(-c3cccc(-c4ccccc4)c3)n2)cc1]

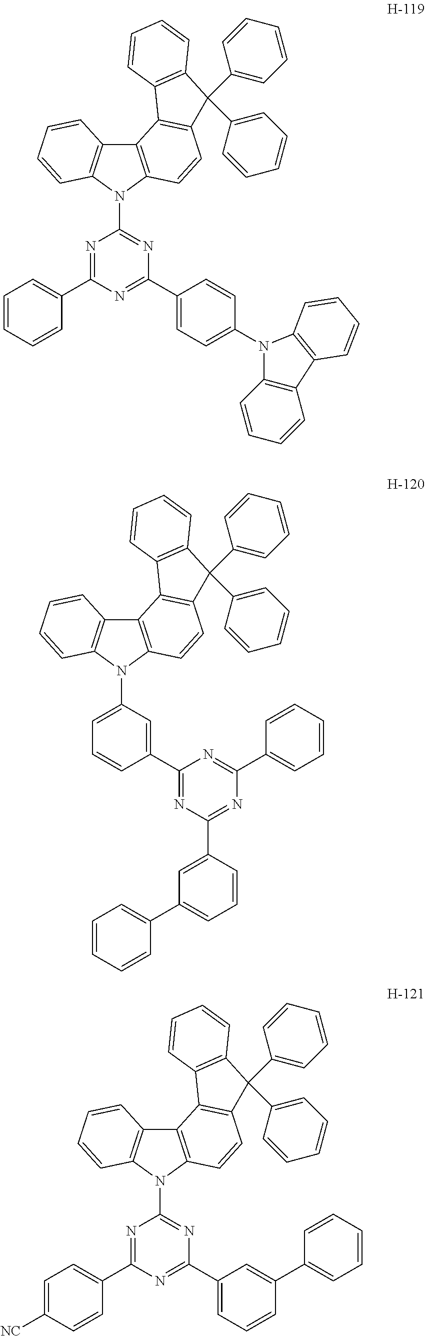 [SMILES: c1ccc(-c2nc(-c3ccc(-n4c5ccccc5c5ccccc54)cc3)nc(-n3c4ccccc4c4c5c(ccc43)C(c3ccccc3)(c3ccccc3)c3ccccc3-5)n2)cc1 ; c1ccc(-c2cccc(-c3nc(-c4ccccc4)nc(-c4cccc(-n5c6ccccc6c6c7c(ccc65)C(c5ccccc5)(c5ccccc5)c5ccccc5-7)c4)n3)c2)cc1 ; N#Cc1ccc(-c2nc(-c3cccc(-c4ccccc4)c3)nc(-n3c4ccccc4c4c5c(ccc43)C(c3ccccc3)(c3ccccc3)c3ccccc3-5)n2)cc1]

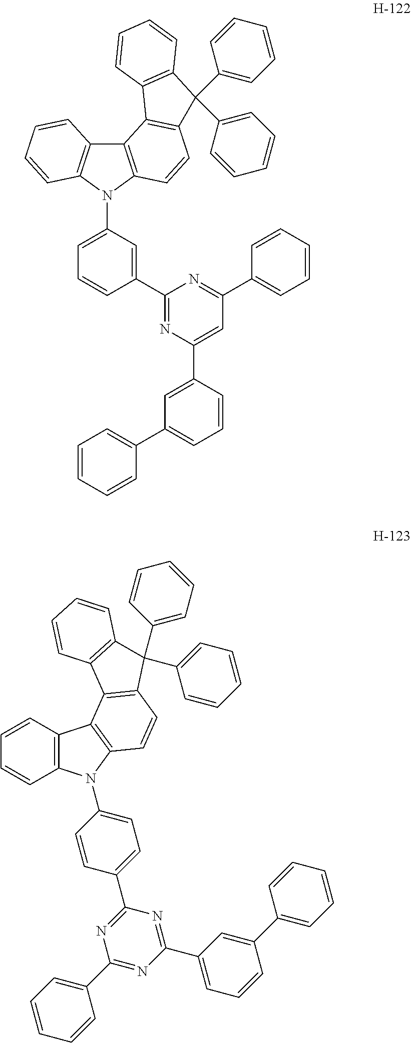 [SMILES: c1ccc(-c2cccc(-c3cc(-c4ccccc4)nc(-c4cccc(-n5c6ccccc6c6c7c(ccc65)C(c5ccccc5)(c5ccccc5)c5ccccc5-7)c4)n3)c2)cc1 ; c1ccc(-c2cccc(-c3nc(-c4ccccc4)nc(-c4ccc(-n5c6ccccc6c6c7c(ccc65)C(c5ccccc5)(c5ccccc5)c5ccccc5-7)cc4)n3)c2)cc1]

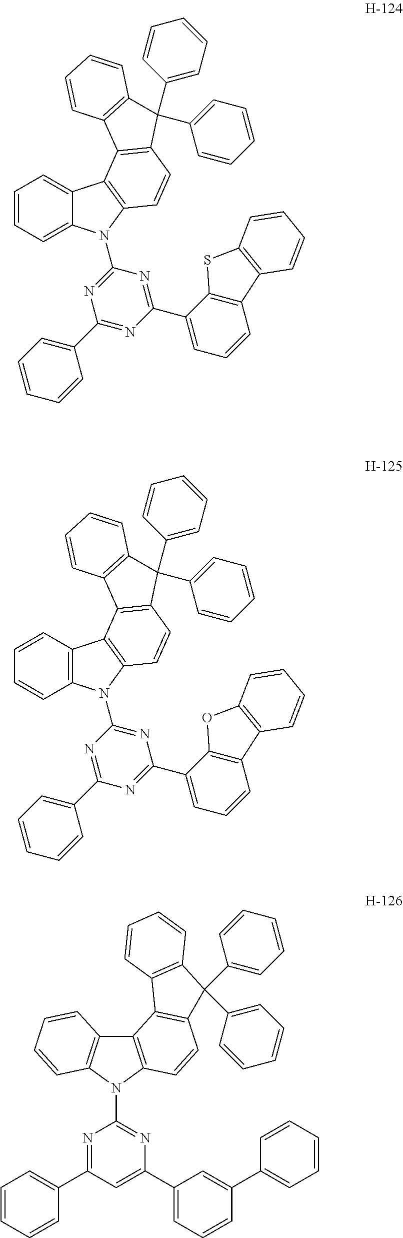 [SMILES: c1ccc(-c2nc(-c3cccc4c3sc3ccccc34)nc(-n3c4ccccc4c4c5c(ccc43)C(c3ccccc3)(c3ccccc3)c3ccccc3-5)n2)cc1 ; c1ccc(-c2nc(-c3cccc4c3oc3ccccc34)nc(-n3c4ccccc4c4c5c(ccc43)C(c3ccccc3)(c3ccccc3)c3ccccc3-5)n2)cc1 ; c1ccc(-c2cccc(-c3cc(-c4ccccc4)nc(-n4c5ccccc5c5c6c(ccc54)C(c4ccccc4)(c4ccccc4)c4ccccc4-6)n3)c2)cc1]

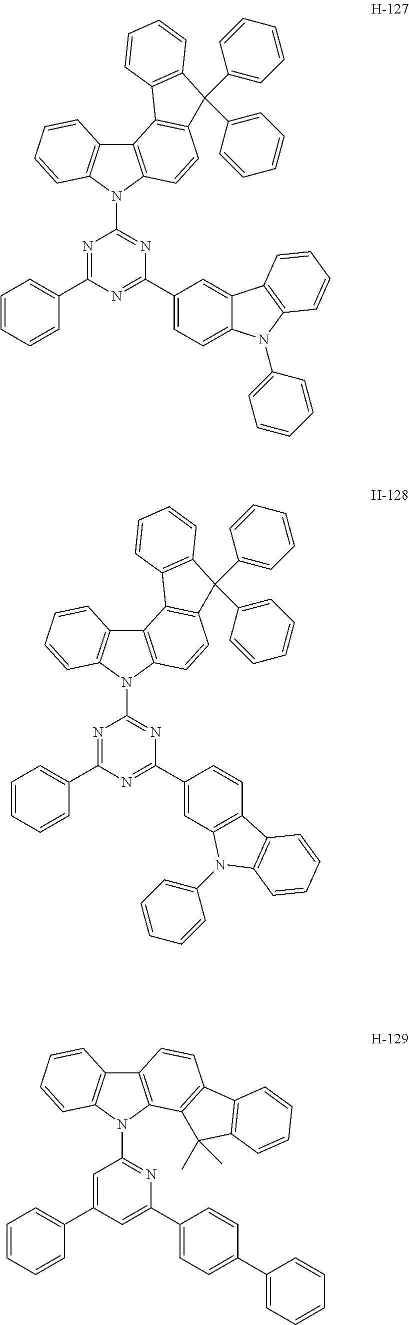 [SMILES: c1ccc(-c2nc(-c3ccc4c(c3)c3ccccc3n4-c3ccccc3)nc(-n3c4ccccc4c4c5c(ccc43)C(c3ccccc3)(c3ccccc3)c3ccccc3-5)n2)cc1 ; c1ccc(-c2nc(-c3ccc4c5ccccc5n(-c5ccccc5)c4c3)nc(-n3c4ccccc4c4c5c(ccc43)C(c3ccccc3)(c3ccccc3)c3ccccc3-5)n2)cc1 ; CC1(C)c2ccccc2-c2ccc3c4ccccc4n(-c4cc(-c5ccccc5)cc(-c5ccc(-c6ccccc6)cc5)n4)c3c21]

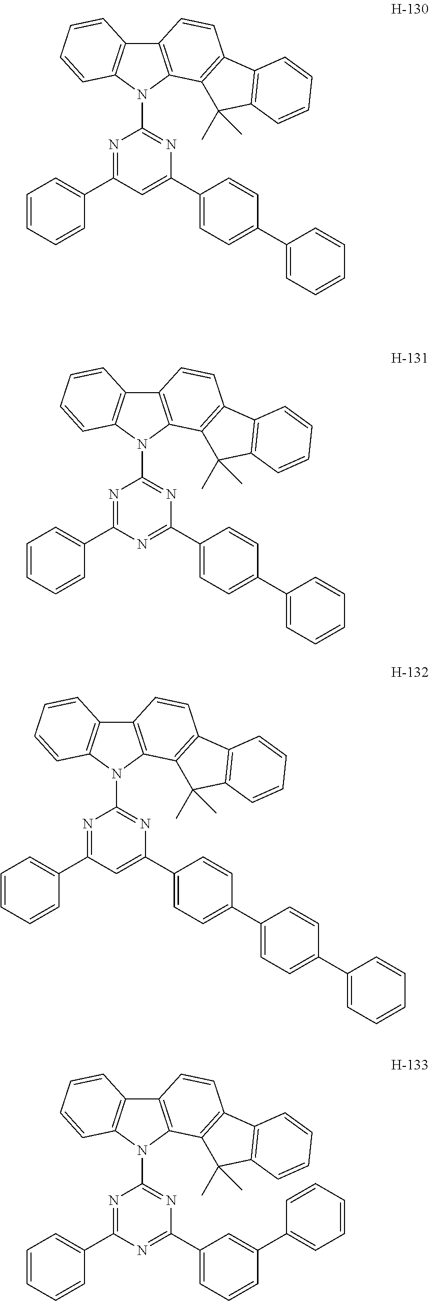 [SMILES: CC1(C)c2ccccc2-c2ccc3c4ccccc4n(-c4nc(-c5ccccc5)cc(-c5ccc(-c6ccccc6)cc5)n4)c3c21 ; CC1(C)c2ccccc2-c2ccc3c4ccccc4n(-c4nc(-c5ccccc5)nc(-c5ccc(-c6ccccc6)cc5)n4)c3c21 ; CC1(C)c2ccccc2-c2ccc3c4ccccc4n(-c4nc(-c5ccccc5)cc(-c5ccc(-c6ccc(-c7ccccc7)cc6)cc5)n4)c3c21 ; CC1(C)c2ccccc2-c2ccc3c4ccccc4n(-c4nc(-c5ccccc5)nc(-c5cccc(-c6ccccc6)c5)n4)c3c21]

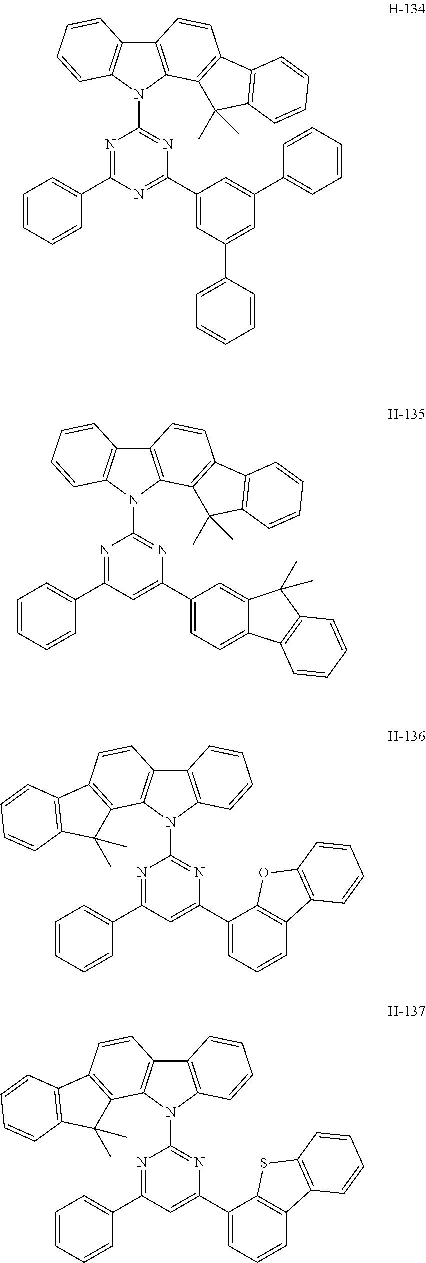 [SMILES: CC1(C)c2ccccc2-c2ccc3c4ccccc4n(-c4nc(-c5ccccc5)nc(-c5cc(-c6ccccc6)cc(-c6ccccc6)c5)n4)c3c21 ; CC1(C)c2ccccc2-c2ccc(-c3cc(-c4ccccc4)nc(-n4c5ccccc5c5ccc6c(c54)C(C)(C)c4ccccc4-6)n3)cc21 ; CC1(C)c2ccccc2-c2ccc3c4ccccc4n(-c4nc(-c5ccccc5)cc(-c5cccc6c5oc5ccccc56)n4)c3c21 ; CC1(C)c2ccccc2-c2ccc3c4ccccc4n(-c4nc(-c5ccccc5)cc(-c5cccc6c5sc5ccccc56)n4)c3c21]

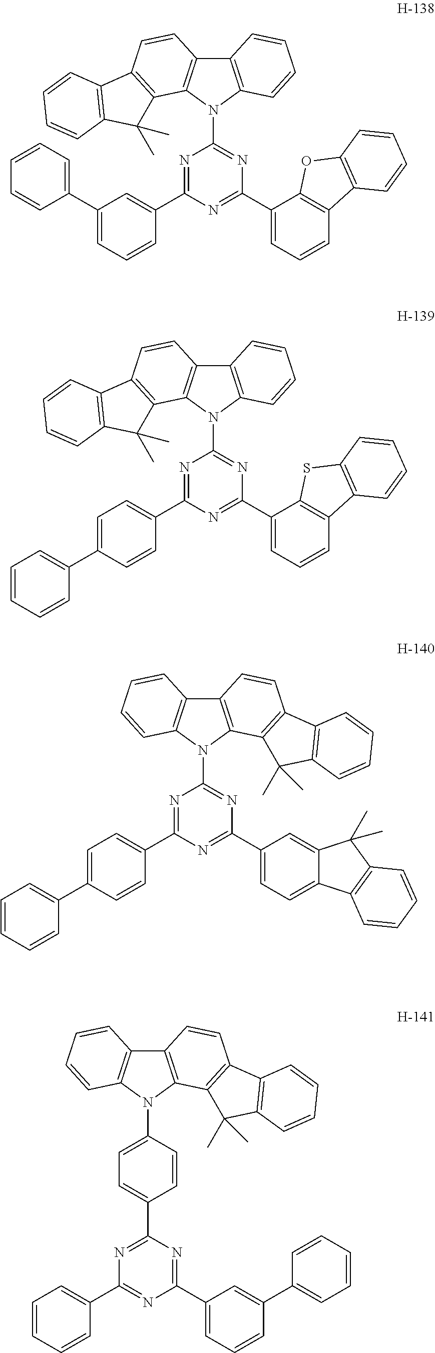 [SMILES: CC1(C)c2ccccc2-c2ccc3c4ccccc4n(-c4nc(-c5cccc(-c6ccccc6)c5)nc(-c5cccc6c5oc5ccccc56)n4)c3c21 ; CC1(C)c2ccccc2-c2ccc3c4ccccc4n(-c4nc(-c5ccc(-c6ccccc6)cc5)nc(-c5cccc6c5sc5ccccc56)n4)c3c21 ; CC1(C)c2ccccc2-c2ccc(-c3nc(-c4ccc(-c5ccccc5)cc4)nc(-n4c5ccccc5c5ccc6c(c54)C(C)(C)c4ccccc4-6)n3)cc21 ; CC1(C)c2ccccc2-c2ccc3c4ccccc4n(-c4ccc(-c5nc(-c6ccccc6)nc(-c6cccc(-c7ccccc7)c6)n5)cc4)c3c21]

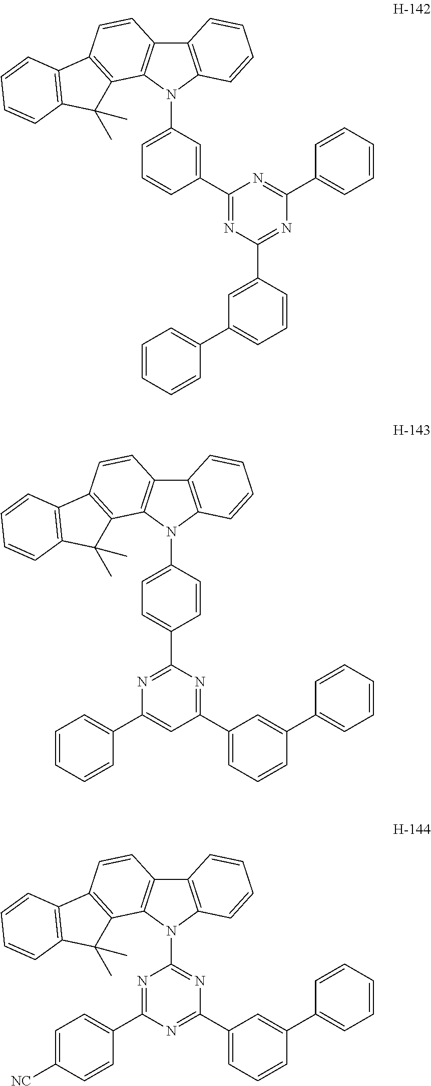 [SMILES: CC1(C)c2ccccc2-c2ccc3c4ccccc4n(-c4cccc(-c5nc(-c6ccccc6)nc(-c6cccc(-c7ccccc7)c6)n5)c4)c3c21 ; CC1(C)c2ccccc2-c2ccc3c4ccccc4n(-c4ccc(-c5nc(-c6ccccc6)cc(-c6cccc(-c7ccccc7)c6)n5)cc4)c3c21 ; CC1(C)c2ccccc2-c2ccc3c4ccccc4n(-c4nc(-c5ccc(C#N)cc5)nc(-c5cccc(-c6ccccc6)c5)n4)c3c21]

8. An organic electroluminescent device comprising the organic electroluminescent compound according to claim 1.
